(19) United States
(12) Patent Application Publication
(10) Pub. No.: US 2024/0141381 A1
(43) Pub. Date: May 2, 2024

(54) COMPOSITIONS AND METHODS FOR TREATING PHENYLKETONURIA

(71) Applicant: American Gene Technologies International Inc., Rockville, MD (US)

(72) Inventors: Tyler Lahusen, Rockville, MD (US); Charles David Pauza, Rockville, MD (US)

(21) Appl. No.: 18/379,015

(22) Filed: Oct. 11, 2023

Related U.S. Application Data
(62) Division of application No. 16/494,196, filed on Sep. 13, 2019, now Pat. No. 11,820,999.
(60) Provisional application No. 62/480,962, filed on Apr. 3, 2017.
(60) Provisional application No. 62/491,118, filed on Apr. 27, 2017.

(51) Int. Cl. C12N 15/86; A61K 35/76; C12N 9/02

(52) U.S. Cl. CPC C12N 15/86; A61K 35/76; C12N 9/0071; C12Y 114/16001; A61K 48/00

(57) ABSTRACT

A lentiviral vector system for expressing a lentiviral particle is disclosed. The lentiviral vector system includes a therapeutic vector. The therapeutic vector comprises a phenylalanine hydroxylase (PAH) sequence for expressing at least one of PAH or a variant thereof, wherein the PAH sequence is truncated.

### CROSS-REFERENCE TO RELATED APPLICATIONS

This application in a divisional application of U.S. application Ser. No. 16/494,196, filed on Sep. 13, 2019 entitled “COMPOSITIONS AND METHODS FOR TREATING PHENYLKETONURIA,” which is a national stage application of PCT/US2018/025733, filed on Apr. 2, 2018 entitled “COMPOSITIONS AND METHODS FOR TREATING PHENYLKETONURIA,” which claims priority to U.S. Provisional Patent Application No. 62/480,962 filed on Apr. 3, 2017 entitled “COMPOSITIONS AND METHODS FOR TREATING PHENYLKETONURIA”, and U.S. Provisional Patent Application No. 62/491,118 filed on Apr. 27, 2017 entitled “COMPOSITIONS AND METHODS FOR TREATING PHENYLKETONURIA,” the disclosures of which are incorporated herein by reference.

### FIELD

Aspects of the invention relate to genetic medicines for treating phenylketonuria (PKU). More specifically, aspects of the invention relate to using lentiviral vectors, including PAH-containing lentiviral vectors, to treat PKU.

### BACKGROUND

Phenylketonuria (PKU) refers to a heterogeneous group of disorders that can lead to increased concentration of phenylalanine in the blood, or hyperphenylalaninemia. Hyperphenylalaninemia can cause intellectual disability, seizures, behavioral problems, and impaired growth and development in affected children if left untreated. The mechanisms by which hyperphenylalaninemia results in intellectual impairment reflect the surprising toxicity of high dose phenylalanine and involve hypomyelination or demyelination of nervous system tissues. PKU has an average reported incidence rate of 1 in 12,000 in North America, affecting males and females equally. The disorder is most common in people of European or Native American Ancestry and reaches much higher levels in the eastern Mediterranean region.

Neurological changes in patients with PKU have been demonstrated within one month of birth, and magnetic resonance imaging (MRI) in adult PKU patients has shown white matter lesions in the brain. The size and number these lesions relate directly to blood phenylalanine concentration. The cognitive profile of adolescents and adults with PKU compared with control subjects can include significantly reduced IQ, processing speed, motor control and inhibitory abilities, and reduced performance on tests of attention.

The majority of PKU is caused by a deficiency of hepatic phenylalanine hydroxylase (PAH). PAH is a multimeric hepatic enzyme that catalyzes the hydroxylation of phenylalanine (Phe) to tyrosine (Tyr) in the presence of molecular oxygen and catalytic amounts of tetrahydrobiopterin (BH₄), its nonprotein cofactor. In the absence of sufficient expression of PAH, phenylalanine levels in the blood increase, leading to hyperphenylalaninemia and harmful side effects in PKU patients. Decreased or absent PAH activity can lead to a deficiency of tyrosine and its downstream products, including melanin, l-thyroxine and the catecholamine neurotransmitters including dopamine.

PKU can be caused by mutations in PAH and/or a defect in the synthesis or regeneration of PAH cofactors (i.e., BH₄). Notably, several PAH mutations have been shown to affect protein folding in the endoplasmic reticulum resulting in accelerated degradation and/or aggregation due to missense mutations (63%) and small deletions 13%) in protein structure that attenuate or largely abolish enzyme catalytic activity.

In general, three major phenotypic groups are used to classify PKU based on blood plasma Phe levels, dietary tolerance to Phe and potential responsiveness to therapy. These groups include classical PKU (Phe >1200 μM), atypical or mild PKU (Phe is 600-1200 μM), and permanent mild hyperphenylalaninemia (HPA, Phe 120-600 μM).

Detection of PKU relies on universal newborn screening (NBS). A drop of blood collected from a heel stick is tested for phenylalanine levels in a screen that is mandatory in all 50 states of the USA.

Currently, lifelong dietary restriction of Phe and BH₄ supplementation are the only two available treatment options for PKU, where early therapeutic intervention is critical to ensure optimal clinical outcomes in affected infants. However, costly medication and special low-protein foods imposes a major burden on patients that can lead to malnutrition, psychosocial or neurocognitive complications notably when these products are not fully covered by private health insurance. Moreover, BH₄ therapy is primarily effective for treatment of mild hyperphenylalaninemia as related to defects in BH₄ biosynthesis, whereas only 20-30% of patients with mild or classical PKU are responsive. Thus, there is an urgent need for new treatment modalities for PKU as an alternative to burdensome Phe-restriction diets. Thus, it would be desirable to develop an alternative method for the treatment of phenylketonuria. Genetic medicines have the potential to effectively treat PKU.

### SUMMARY OF THE INVENTION

In an aspect, a viral vector is disclosed. The viral vector comprises a phenylalanine hydroxylase (PAH) sequence for expressing at least one of PAH or a variant thereof, wherein the PAH sequence is truncated. In embodiments, the PAH sequence is truncated at a 3′ untranslated region (UTR) of the sequence. In embodiments, the PAH sequence comprises at least one of 80%, 85%, 90%, 95%, or 100% identity with at least one of SEQ ID NO: 2, SEQ ID NO: 3, or SEQ ID NO: 4.

In embodiments, the viral vector further comprises at least one small RNA sequence that is capable of binding to at least one pre-determined complementary mRNA sequence. In embodiments, the at least one pre-determined complementary mRNA sequence comprises a full-length 3′ untranslated region (UTR). In embodiments, the at least one pre-determined complementary mRNA sequence is a PAH mRNA sequence. In embodiments, the at least one small RNA sequence comprises a shRNA. In embodiments, the at least one small RNA sequence comprises a sequence having at least one of 80%, 85%, 90%, 95%, or 100% identity with at least one of SEQ ID NO: 5 or SEQ ID NO: 6. In embodiments, the at least one small RNA sequence is under the control of a first promoter, and wherein the PAH sequence is under the control of a second promoter. In embodiments, the first promoter comprises a H1 promoter. In embodiments, the second promoter comprises a liver-specific promoter. In embodiments, the liver-specific promoter comprises a hAAT promoter.

In another aspect, a viral vector is disclosed. The viral vector comprises a phenylalanine hydroxylase (PAH) sequence for expressing at least one of PAH or a variant thereof, and at least one small RNA sequence that is capable of binding to at least one pre-determined complementary mRNA sequence. In embodiments, the PAH sequence comprises at least one of 80%, 85%, 90%, 95%, or 100% identity with SEQ ID NO: 1.

In another aspect, a lentiviral particle produced by a packaging cell and capable of infecting a target cell is disclosed. The lentiviral particle comprises an envelope protein capable of infecting a target cell, and the viral vector comprises a phenylalanine hydroxylase (PAH) sequence for expressing at least one of PAH or a variant thereof, and at least one small RNA sequence that is capable of binding to at least one pre-determined complementary mRNA sequence. In embodiments, the target cell is at least one of a hepatic cell, a muscle cell, an epithelial cell, an endothelial cell, a neural cell, a neuroendocrine cell, an endocrine cell, a lymphocyte, a myeloid cell, a cell present within a solid organ, or cell of a hematopoietic lineage, a hematopoietic stem cell, or a precursor hematopoietic stem cell.

In another aspect, a method of treating phenylketonuria (PKU) in a subject is disclosed. The method comprises administering to the subject a therapeutically effective amount of a lentiviral particle produced by a packaging cell and capable of infecting a target cell, wherein the lentiviral particle comprises an envelope protein capable of infecting a target cell, and a viral vector comprising a phenylalanine hydroxylase (PAH) sequence for expressing at least one of PAH or a variant thereof, and at least one small RNA sequence that is capable of binding to at least one pre-determined complementary mRNA sequence.

In embodiments, the therapeutically effective amount of the lentiviral particle comprises a plurality of single doses of the lentiviral particle. In embodiments, the therapeutically effective amount of the lentiviral particle comprises a single dose of the lentiviral particle. In embodiments, the subject is in utero. In embodiments, the method further comprises diagnosing a PKU genotype in the subject that correlates with a PKU phenotype. In embodiments, the diagnosing occurs during prenatal screening of the subject. In embodiments, the diagnosing occurs prior to the administering.

In another aspect, use of a therapeutically effective amount of a lentiviral particle for treatment of phenylketonuria (PKU) in a subject is disclosed. The lentiviral particle is produced by a packaging cell, is capable of infecting a target cell, and comprises an envelope protein capable of infecting a target cell, and a viral vector. In embodiments, the viral vector comprises a phenylalanine hydroxylase (PAH) sequence for expressing at least one of PAH or a variant thereof, wherein the PAH sequence is truncated. In embodiments, the viral vector comprises a phenylalanine hydroxylase (PAH) sequence for expressing at least one of PAH or a variant thereof, and at least one small RNA sequence that is capable of binding to at least one pre-determined complementary mRNA sequence.

Other aspects and advantages of the inventions described herein will become apparent from the following detailed description, taken in conjunction with the accompanying drawings, which illustrate by way of example the aspects of the inventions.

### BRIEF DESCRIPTION OF THE DRAWINGS

FIG. 1 depicts an exemplary 3-vector lentiviral vector system in a circularized form.

FIG. 2 depicts an exemplary 4-vector lentiviral vector system in a circularized form.

FIG. 3 depicts an exemplary 3-vector adeno-associated viral vector system in a circularized form.

FIG. 4 depicts: (A) a linear map of a lentiviral vector expressing PAH; and (B) a linear map of a lentiviral vector expressing a PAH shRNA sequence and a PAH sequence.

FIG. 5 depicts a phenylalanine hydroxylase open reading frame including complete 5′ and 3′ UTRs.

FIG. 6 depicts a phenylalanine hydroxylase open reading frame including a complete 5′ UTR and a truncated 3′ UTR.

FIG. 7 depicts immunoblot data comparing levels of expression for human and mouse PAH genes.

FIG. 8 depicts data demonstrating lentivirus-delivered expression of hPAH with or without the 3′ UTR region in Hepa1-6 cells:

FIG. 9 depicts results of a lentiviral vector expressing hPAH with a truncated 3′ UTR in Hepa1-6 cells.

FIG. 10 depicts data demonstrating expression of codon-optimized hPAH with or without WPRE in mouse Hepa1-6 cells.

FIG. 11 depicts data demonstrating that shPAH-1 and shPAH-2 reduces hPAH expression in human Hep3B cells.

FIG. 12 depicts data demonstrating shPAH-1 suppression of endogenous hPAH and hAAT-hPAH-3′UTR₂₈₉ in Hep3B cells.

FIG. 13 depicts data demonstrating that shPAH-2 suppresses endogenous hPAH but not hAAT-hPAH-3′UTR₂₈₉ in HepG2 cells.

FIG. 14 depicts data demonstrating results of treating a Pah(enu2) mouse with hAAT-PAH-UTR. FIG. 14A depicts change in weight over 8 weeks for the groups depicted therein. FIG. 14B depicts change in weight over 8 weeks for the groups depicted therein. FIG. 14C depicts change in weight over 8 weeks for the groups depicted therein. FIG. 14D depicts levels of phenylalanine over 1 month post-treatment.

FIG. 15 depicts data demonstrating lentiviral-delivered expression of the human PAH gene using hAAT and CMV promoters in Hepa1-6 mouse hepatoma cells.

FIG. 16 depicts demonstrating lentivirus-delivered expression of hPAH using expression constructs with the hAAT promoter and liver-specific enhancer element ApoE (1), ApoE (2), or prothrombin in mouse Hepa1-6 cells.

### DETAILED DESCRIPTION

### Overview of the Disclosure

The present disclosure relates to therapeutic vectors and delivery of the same to cells. In embodiments, the therapeutic vectors include PAH sequences or variants thereof. In embodiments, the therapeutic vectors also include a small RNA that targets host (endogenous) PAH expression.

### Definitions and Interpretation

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g.: Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual; Cold Spring Harbor Laboratory Press, Cold Spring Harbor, N.Y. (1998); and Coligan et al., Short Protocols in Protein Science, Wiley, John & Sons, Inc. (2003). Any enzymatic reactions or purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

As used in the description and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. It also is to be understood, although not always explicitly stated, that the reagents described herein are merely exemplary and that equivalents of such are known in the art.

As used herein, the term “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The terms “administration of” or “administering” an active agent should be understood to mean providing an active agent to the subject in need of treatment in a form that can be introduced into that individual's body in a therapeutically useful form and therapeutically effective amount.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

As used herein, the terms “expression”, “expressed”, or “encodes” refers to the process by which polynucleotides are transcribed into mRNA and/or the process by which the transcribed mRNA is subsequently being translated into peptides, polypeptides, or proteins. Expression may include splicing of the mRNA in a eukaryotic cell or other forms of post-transcriptional modification or post-translational modification.

As used herein, the term “phenylketonuria”, which is also referred to herein as “PKU”, refers to the chronic deficiency of phenylalanine hydroxylase, as well as all symptoms related thereto including mild and classical forms of disease. Treatment of “phenylketonuria”, therefore, may relate to treatment for all or some of the symptoms associated with PKU.

As used herein, the term “phenylalanine hydroxylase” may also be referred to herein as PAN. Human PAH may also be referred to herein as hPAH. Mouse PAH may also be referred to herein as mPAH.

As used herein, the term “shPAH” refers to a small hairpin RNA targeting PAN.

As used herein, the term “hAAT-hPAH-3′UTR₂₈₉” may also be referred to herein as U₂₈₉, or generally as transgene-expressed truncated hPAH 3′UTR, or generally a truncated 3′ UTR

As used herein, the term “hAAT-hPAH-3′UTR₂₃₈” may also be referred to herein as U₂₃₈, or generally as transgene-expressed truncated hPAH 3′UTR, or generally a truncated 3′ UTR.

As used herein, the term “wild type hPAH” may also be referred to herein as “endogenous PAH” or “full-length PAN”.

As used herein, the term truncated may also be referred to herein as “shortened” or “without”.

As used herein, the term variant may also be referred to herein as analog or variation. A variant refers to any substitution, deletion, or addition to a nucleotide sequence.

As used herein, the term “genetic medicine” or “genetic medicines” refers generally to therapeutics and therapeutic strategies that focus on genetic targets to treat a clinical disease or manifestation. The term “genetic medicine” encompasses gene therapy and the like.

As used herein, the terms “individual,” “subject,” and “patient” are used interchangeably herein, and refer to any individual mammal subject, e.g., bovine, canine, feline, equine, or human.

As used herein, the term “LV” refers generally to “lentivirus”. As an example, reference to “LV-shPAH” is reference to a lentivirus that expresses a shRNA that targets PAH.

As used herein, the term “packaging cell line” refers to any cell line that can be used to express a lentiviral particle.

As used herein, the term “percent identity”, in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules provided in the disclosure. BLAST protein searches can be performed with the XBLAST program, score=50, word length=3 to obtain amino acid sequences homologous to the protein molecules of the disclosure. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

As used herein, the term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the term “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

As used herein, the term “SEQ ID NO” is synonymous with the term “Sequence ID No.”

As used herein, “small RNA” refers to non-coding RNA that are generally about 200 nucleotides or less in length and possess a silencing or interference function. In other embodiments, the small RNA is about 175 nucleotides or less, about 150 nucleotides or less, about 125 nucleotides or less, about 100 nucleotides or less, or about 75 nucleotides or less in length. Such RNAs include microRNA (miRNA), small interfering RNA (siRNA), double stranded RNA (dsRNA), and short hairpin RNA (shRNA). “Small RNA” of the disclosure should be capable of inhibiting or knocking-down gene expression of a target gene, generally through pathways that result in the destruction of the target gene mRNA.

As used herein, the term “therapeutically effective amount” refers to a sufficient quantity of the active agents of the present disclosure, in a suitable composition, and in a suitable dosage form to treat or prevent the symptoms, progression, or onset of the complications seen in patients suffering from a given ailment, injury, disease, or condition. The therapeutically effective amount will vary depending on the state of the patient's condition or its severity, and the age, weight, etc., of the subject to be treated. A therapeutically effective amount can vary, depending on any of a number of factors, including, e.g., the route of administration, the condition of the subject, as well as other factors understood by those in the art.

As used herein, the term “therapeutic vector” includes, without limitation, reference to a lentiviral vector or an adeno-associated viral (AAV) vector. Additionally, as used herein with reference to the lentiviral vector system, the term “vector” is synonymous with the term “plasmid”. For example, the 3-vector and 4-vector systems, which include the 2-vector and 3-vector packaging systems, can also be referred to as 3-plasmid and 4-plasmid systems.

As used herein, the term “treatment” or “treating” generally refers to an intervention in an attempt to alter the natural course of the subject being treated, and can be performed either for prophylaxis or during the course of clinical pathology. Desirable effects include, but are not limited to, preventing occurrence or recurrence of disease, alleviating symptoms, suppressing, diminishing or inhibiting any direct or indirect pathological consequences of the disease, ameliorating or palliating the disease state, and causing remission or improved prognosis. The particular treatment thus will depend on the disease state to be targeted and the current or future state of medicinal therapies and therapeutic approaches. A treatment may have associated toxicities.

### Description of Aspects and Embodiments of the Disclosure

In an aspect of the present disclosure, a viral vector is disclosed. The viral vector comprises a therapeutic cargo portion, wherein the therapeutic cargo portion comprises a PAH sequence or a variant thereof. In embodiments, the PAH sequence or the variant is truncated. In embodiments, the portion of the PAN sequence or the variant thereof that is truncated is the 3′ untranslated region (UTR) of the PAN sequence or the variant thereof. In embodiments, the PAN sequence or the variant thereof comprises a sequence having at least 80%, or at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with:

(SEQ ID NO: 1)
ATGTCCACTGCGGTCCTGGAAAACCCAGGCTTGGGCAGGAAACTCTCTGA
CTTTGGACAGGAAACAAGCTATATTGAAGACAACTGCAATCAAAATGGTG
CCATATCACTGATCTTCTCACTCAAAGAAGAAGTTGGTGCATTGGCCAAA
GTATTGCGCTTATTTGAGGAGAATGATGTAAACCTGACCCACATTGAATC
TAGACCTTCTCGTTTAAAGAAAGATGAGTATGAATTTTTCACCCATTTGG
ATAAACGTAGCCTGCCTGCTCTGACAAACATCATCAAGATCTTGAGGCAT
GACATTGGTGCCACTGTCCATGAGCTTTCACGAGATAAGAAGAAAGACAC
AGTGCCCTGGTTCCCAAGAACCATTCAAGAGCTGGACAGATTTGCCAATC
AGATTCTCAGCTATGGAGCGGAACTGGATGCTGACCACCCTGGTTTTAAA
GATCCTGTGTACCGTGCAAGACGGAAGCAGTTTGCTGACATTGCCTACAA
CTACCGCCATGGGCAGCCCATCCCTCGAGTGGAATACATGGAGGAAGAAA
AGAAAACATGGGGCACAGTGTTCAAGACTCTGAAGTCCTTGTATAAAACC
CATGCTTGCTATGAGTACAATCACATTTTTCCACTTCTTGAAAAGTACTG
TGGCTTCCATGAAGATAACATTCCCCAGCTGGAAGACGTTTCTCAATTCC
TGCAGACTTGCACTGGTTTCCGCCTCCGACCTGTGGCTGGCCTGCTTTCC
TCTCGGGATTTCTTGGGTGGCCTGGCCTTCCGAGTCTTCCACTGCACACA
GTACATCAGACATGGATCCAAGCCCATGTATACCCCCGAACCTGACATCT
GCCATGAGCTGTTGGGACATGTGCCCTTGTTTTCAGATCGCAGCTTTGCC
CAGTTTTCCCAGGAAATTGGCCTTGCCTCTCTGGGTGCACCTGATGAATA
CATTGAAAAGCTCGCCACAATTTACTGGTTTACTGTGGAGTTTGGGCTCT
GCAAACAAGGAGACTCCATAAAGGCATATGGTGCTGGGCTCCTGTCATCC
TTTGGTGAATTACAGTACTGCTTATCAGAGAAGCCAAAGCTTCTCCCCCT
GGAGCTGGAGAAGACAGCCATCCAAAATTACACTGTCACGGAGTTCCAGC
CCCTGTATTACGTGGCAGAGAGTTTTAATGATGCCAAGGAGAAAGTAAGG
AACTTTGCTGCCACAATACCTCGGCCCTTCTCAGTTCGCTACGACCCATA
CACCCAAAGGATTGAGGTCTTGGACAATACCCAGCAGCTTAAGATTTTGG
CTGATTCCATTAACAGTGAAATTGGAATCCTTTGCAGTGCCCTCCAGAAA
ATAAAGTAA;
(SEQ ID NO: 2)
ATGAGCACAGCTGTGTTGGAAAATCCTGGGCTGGGCCGTAAGCTTTCCGA
TTTCGGCCAGGAGACTTCATACATTGAGGACAACTGCAACCAGAATGGGG
CCATTTCTTTGATCTTCAGTCTCAAAGAAGAGGTAGGCGCTCTGGCTAAG
GTCCTGAGGCTGTTTGAGGAAAATGACGTGAATCTGACACACATTGAGTC
TAGGCCTTCCCGACTTAAGAAGGATGAGTATGAGTTCTTCACACACCTGG
ACAAACGATCTCTCCCAGCACTGACCAATATCATCAAGATTCTCAGGCAT
GATATCGGTGCCACGGTCCACGAACTTTCACGCGATAAGAAGAAAGACAC
AGTTCCCTGGTTCCCGAGAACCATTCAGGAACTGGATAGGTTTGCCAATC
AGATTCTGAGCTATGGGGCAGAGTTGGATGCCGACCATCCAGGCTTCAAA
GACCCCGTATATCGGGCTCGGAGAAAGCAGTTTGCAGACATCGCTTACAA
TTACAGGCATGGACAGCCCATCCCTAGAGTGGAGTACATGGAAGAAGGCA
AGAAAACCTGGGGAACGGTGTTTAAGACCCTCAAAAGCCTGTATAAGACC
CACGCGTGTTATGAGTACAACCACATTTTCCCATTGCTGGAGAAGTACTG
TGGCTTTCACGAGGACAACATCCCTCAACTGGAGGATGTTTCACAGTTCC
TTCAGACTTGCACTGGTTTCCGCCTTCGACCTGTGGCTGGGCTGCTTAGC
TCACGGGACTTCCTGGGAGGCCTGGCCTTCAGAGTCTTTCACTGCACTCA
GTACATTCGGCATGGCTCTAAGCCAATGTACACCCCTGAACCGGATATAT
GCCACGAGCTGTTGGGACATGTGCCCCTGTTTTCTGATCGCAGCTTTGCC
CAGTTTTCCCAGGAGATTGGCCTGGCAAGTCTTGGTGCGCCTGATGAGTA
CATCGAGAAGCTCGCGACAATCTACTGGTTCACCGTGGAATTTGGACTCT
GCAAACAAGGGGACTCTATCAAAGCCTACGGAGCAGGACTCCTCTCCAGC
TTCGGTGAACTGCAGTATTGTCTGTCCGAGAAACCCAAACTCTTGCCCCT
GGAACTGGAAAAGACTGCCATCCAAAACTATACTGTCACGGAATTTCAGC
CACTGTATTATGTGGCTGAATCCTTTAACGATGCCAAGGAGAAGGTCCGT
AATTTTGCTGCCACAATACCACGCCCCTTCAGCGTGAGATACGACCCGTA
TACACAACGGATAGAGGTTCTGGACAACACCCAGCAACTGAAAATTCTGG
CAGACAGTATAAACAGCGAAATAGGGATCCTCTGTAGTGCCCTGCAGAAA
ATCAAATGA;
(SEQ ID NO: 3)
AGCCATGGACAGAATGTGGTCTGTCAGCTGTGAATCTGTTGATGGAGATC
CAACTATTTCTTTCATCAGAAAAAGTCCGAAAAGCAAACCTTAATTTGAA
ATAACAGCCTTAAATCCTTTACAAGATGGAGAAACAACAAATAAGTCAAA
ATAATCTGAAATGACAGGATATGAGTACATACTCAAGAGCATAATGGTAA
ATCTTTTGGGGTCATCTTTGATTTAGAGATGATAATCCCATACTCTCAAT
TGAGTTAAATCAGTAATCTGTCGCATTTCATCAAGATTAATTAAAATTTG
GGACCTGCTTCATTCAAGCTTCATATATGCTTTGCAGAGAACTCATAAAG
GAGCATATAAGGCTAAATGTAAAACCCAAGACTGTCATTAGAATTGAATT
ATTGGGCTTAATATAAATCGTAACCTATGAAGTTTATTTTTTATTTTAGT
TAACTATGATTCCAATTACTACTTTGTTATTGTACCTAAGTAAATTTTCT
TTAAGTCAGAAGCCCATTAAAATAGTTACAAGCATTGAACTTCTTTAGTA
TTATATTAATATAAAAACATTTTTGTATGTTTTATTGTAATCATAAATAC
TGCTGTATAAGGTAATAAAACTCTGCACCTAATCCCCATAACTTCCAGTA
TCATTTTCCAATTAATTATCAAGTCTGTTTTGGGAAACACTTTGAGGACA
TTTATGATGCAGCAGATGTTGACTAAAGGCTTGGTTGGTAGATATTCAGG
AAATGTTCACTGAATAAATAAGTAAATACATTATTGAAAAGCAAATCTGT
ATAAATGTGAAATTTTTATTTGTATTAGTAATAAAACATTAGTAGTTTAA
ACAAAAAAAAAAAAAAAAAAAAAAAAAAAAAAACTCGACTCTAGATT;
or
(SEQ ID NO: 4)
AGCCATGGACAGAATGTGGTCTGTCAGCTGTGAATCTGTTGATGGAGATC
CAACTATTTCTTTCATCAGAAAAAGTCCGAAAAGCAAACCTTAATTTGAA
ATAACAGCCTTAAATCCTTTACAAGATGGAGAAACAACAAATAAGTCAAA
ATAATCTGAAATGACAGGATATGAGTACATACTCAAGAGCATAATGGTAA
ATCTTTTGGGGTCATCTTTGATTTAGAGATGATAATCCCATACTCTCAAT
TGAGTTAAATCAGTAATCTGTCGCATTTCATCAAGATTA.

In embodiments, variants can be made to any of the above-described sequences. In embodiments, the PAH sequence or the variant thereof comprises (SEQ ID NO: 1), (SEQ ID NO: 2), (SEQ ID NO: 3), or (SEQ ID NO: 4).

In embodiments, the therapeutic cargo portion comprises at least one small RNA sequence that is capable of binding to at least one pre-determined complementary mRNA sequence. In embodiments, the at least one small RNA sequence targets a complementary mRNA sequence that contains a full-length UTR. In embodiments, the at least one small RNA sequence does not target a complementary mRNA sequence that contains a truncated UTR. In embodiments, the truncated UTR can include any of the truncated sequences identified herein or any variants thereof. In embodiments, the at least one pre-determined complementary mRNA sequence is a PAH mRNA sequence. In embodiments, the at least one small RNA sequence comprises a shRNA. In embodiments, the at least one small RNA sequence is under the control of a first promoter, and the PAH sequence or the variant thereof is under the control of a second promoter. In embodiments, the first promoter comprises a H1 promoter. In embodiments, the second promoter comprises a liver-specific promoter. In embodiments, the liver-specific promoter comprises a hAAT promoter. In embodiments, the at least one small RNA sequence comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95% or more percent identity with:

(SEQ ID NO: 5)
TCGCATTTCATCAAGATTAATCTCGAGATTAATCTTGATGAAATGCGATT
TTT;
or
(SEQ ID NO: 6)
ACTCATAAAGGAGCATATAAGCTCGAGCTTATATGCTCCTTTATGAGTTT
TTT.

In embodiments, variants can be made of the above-described sequences. In embodiments, the at least one small RNA sequence comprises: (SEQ ID NO: 5), or (SEQ ID NO: 6). In embodiments, the viral vector is a lentiviral vector or an adeno-associated viral vector.

In another aspect, a lentiviral particle capable of infecting a target cell is disclosed. The lentiviral particle comprises an envelope protein optimized for infecting the target cell; and further comprises a viral vector as detailed herein. In embodiments, the target cell is a hepatic cell.

In another aspect, a method of treating PKU in a subject is disclosed. The method comprises administering to the subject a therapeutically effective amount of a lentiviral particle as detailed herein.

In another aspect, a method of preventing PKU in a subject is disclosed. The method comprises administering to the subject a therapeutically effective amount of the lentiviral particle as detailed herein. In embodiments, the therapeutically effective amount of the lentiviral particle comprises a plurality of single doses of the lentiviral particle. In embodiments, the therapeutically effective amount of the lentiviral particle comprises a single dose of the lentiviral particle. In embodiments, the method comprises administering to the subject therapeutically effective amounts of a first lentiviral particle and a second lentiviral particle comprising a viral vector. In embodiments, the first lentiviral particle comprises a PAH sequence or a variant thereof, and the second lentiviral particle comprises at least one small RNA sequence that is capable of binding to at least one pre-determined complementary mRNA sequence.

In another aspect, a method of treating or preventing PKU in a subject is disclosed. In embodiments, the subject is in utero. In embodiments, the method of treating or preventing PKU further comprises diagnosing a PKU genotype in the subject that correlates with a PKU phenotype. In embodiments, the method of treating or preventing PKU comprises diagnosis during prenatal screening of the subject. However, in embodiments, a subject may be diagnosed at any time prior to or after treatment.

Other aspects and advantages of the inventions described herein will become apparent from the following detailed description, taken in conjunction with the accompanying drawings, which illustrate by way of example the aspects of the inventions.

### Phenylketonuria

PKU is believed to be caused by mutations of PAH and/or a defect in the synthesis or regeneration of PAH cofactors (i.e.; BH₄). Notably, several PAH mutations have been shown to affect protein folding in the endoplasmic reticulum resulting in accelerated degradation and/or aggregation due to missense mutations (63%) and small deletions (13%) in protein structure that attenuates or largely abolishes enzyme catalytic activity. As there are numerous mutations that can affect the functionality of PAH, an effective therapeutic approach for treating PKU may address the aberrant PAH and/or a mode by which replacement PAH can be administered.

In general, three major phenotypic groups are classified in PKU based on Phe levels measured at diagnosis, dietary tolerance to Phe and potential responsiveness to therapy. These groups include classical PKU (Phe >1200 μM), atypical or mild PKU (Phe is 600-1200 μM), and permanent mild hyperphenylalaninemia (HPA, Phe 120-600 μM).

Detection of PKU typically occurs during universal newborn screening (NBS). A drop of blood collected from a heel stick is tested for phenylalanine levels. NBS is mandatory in all 50 states of the USA.

### Genetic Medicines

Genetic medicine includes reference to viral vectors that are used to deliver genetic constructs to host cells for the purposes of disease therapy or prevention.

Genetic constructs can include, but are not limited to, functional genes or portions of genes to correct or complement existing defects, DNA sequences encoding regulatory proteins, DNA sequences encoding regulatory RNA molecules including antisense, short homology RNA, long non-coding RNA, small interfering RNA or others, and decoy sequences encoding either RNA or proteins designed to compete for critical cellular factors to alter a disease state. Genetic medicine involves delivering these therapeutic genetic constructs to target cells to provide treatment or alleviation of a particular disease.

By delivering a functional PAH gene to the liver in vivo, its activity may be reconstituted, leading to normal clearance of Phe in the blood therefore eliminating the need for dietary restrictions or frequent enzyme replacement therapies. The effect of this therapeutic approach may be improved by the targeting of a shRNA against endogenous PAN. In an aspect of the disclosure, a functional PAH gene or a variant thereof can be delivered in utero if a fetus has been identified as being at risk of having a PKU genotype, especially in cases where the parental genotypes are known. Treatment may occur in vivo or in utero. In embodiments, the diagnostic step may be carried out to determine whether the fetus is at risk for a PKU phenotype. If the diagnostic step determines that the fetus is at risk for a PKU phenotype, then the fetus may be treated with the genetic medicines detailed herein.

### Therapeutic Vectors

A lentiviral virion (particle) in accordance with various aspects and embodiments herein is expressed by a vector system encoding the necessary viral proteins to produce a virion (viral particle). In various embodiments, one vector containing a nucleic acid sequence encoding the lentiviral pol proteins is provided for reverse transcription and integration, operably linked to a promoter. In another embodiment, the pol proteins are expressed by multiple vectors. In other embodiments, vectors containing a nucleic acid sequence encoding the lentiviral Gag proteins for forming a viral capsid, operably linked to a promoter, are provided. In embodiments, this gag nucleic acid sequence is on a separate vector than at least some of the pol nucleic acid sequence. In other embodiments, the gag nucleic acid is on a separate vector from all the pol nucleic acid sequences that encode pol proteins.

Numerous modifications can be made to the vectors herein, which are used to create the particles, to further minimize the chance of obtaining wild type revertants. These include, but are not limited to deletions of the U3 region of the LTR, tat deletions and matrix (MA) deletions. In embodiments, the gag, pol and env vector(s) do not contain nucleotides from the lentiviral genome that package lentiviral RNA, referred to as the lentiviral packaging sequence.

The vector(s) forming the particle preferably do not contain a nucleic acid sequence from the lentiviral genome that expresses an envelope protein. Preferably, a separate vector that contains a nucleic acid sequence encoding an envelope protein operably linked to a promoter is used. This env vector also does not contain a lentiviral packaging sequence. In one embodiment the env nucleic acid sequence encodes a lentiviral envelope protein.

In another embodiment the envelope protein is not from the lentivirus, but from a different virus. The resultant particle is referred to as a pseudotyped particle. By appropriate selection of envelopes one can “infect” virtually any cell. For example, one can use an env gene that encodes an envelope protein that targets an endocytic compartment such as that of the influenza virus, VSV-G, alpha viruses (Semliki forest virus, Sindbis virus), arenaviruses (lymphocytic choriomeningitis virus), flaviviruses (tick-borne encephalitis virus, Dengue virus, hepatitis C virus, GB virus), rhabdoviruses (vesicular stomatitis virus, rabies virus), paramyxoviruses (mumps or measles) and orthomyxoviruses (influenza virus). Other envelopes that can preferably be used include those from Moloney Leukemia Virus such as MLV-E, MLV-A and GALV. These latter envelopes are particularly preferred where the host cell is a primary cell. Other envelope proteins can be selected depending upon the desired host cell.

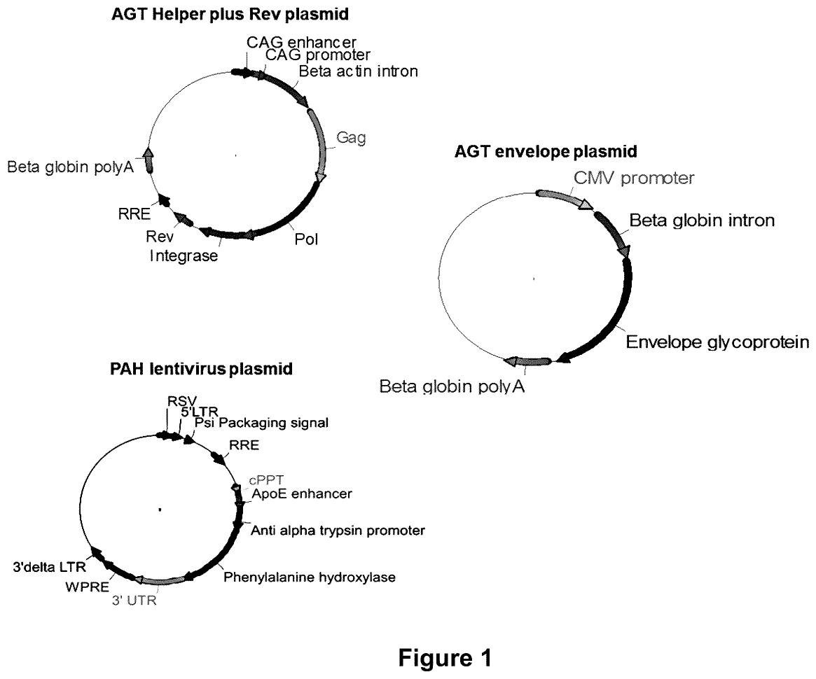

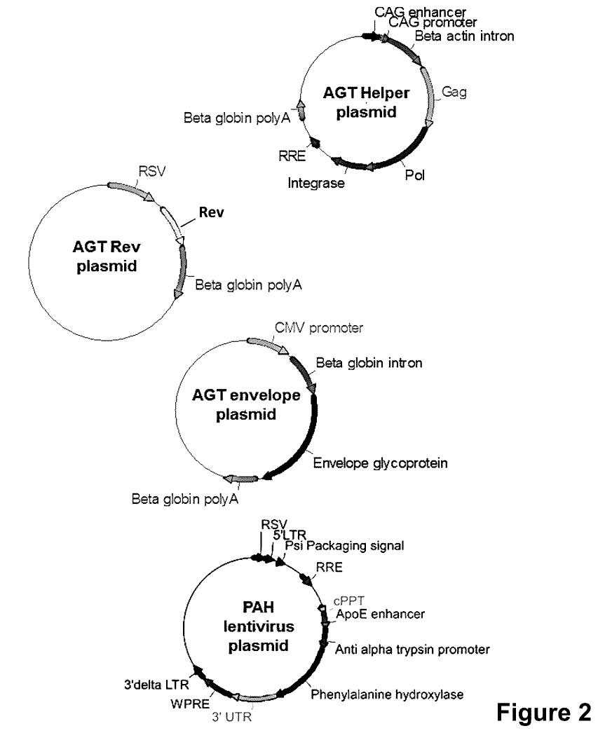

Lentiviral vector systems as provided herein typically include at least one helper plasmid comprising at least one of a gag, pol, or rev gene. Each of the gag, pol and rev genes may be provided on individual plasmids, or one or more genes may be provided together on the same plasmid. In one embodiment, the gag, pol, and rev genes are provided on the same plasmid (e.g., FIG. 1). In another embodiment, the gag and pol genes are provided on a first plasmid and the rev gene is provided on a second plasmid (e.g., FIG. 2). Accordingly, both 3-vector and 4-vector systems can be used to produce a lentivirus as described herein. In embodiments, the therapeutic vector, at least one envelope plasmid and at least one helper plasmid are transfected into a packaging cell, for example a packaging cell line. A non-limiting example of a packaging cell line is the 293T/17 HEK cell line. When the therapeutic vector, the envelope plasmid, and at least one helper plasmid are transfected into the packaging cell line, a lentiviral particle is ultimately produced.

In another aspect, a lentiviral vector system for expressing a lentiviral particle is disclosed. The system includes a lentiviral vector as described herein; an envelope plasmid for expressing an envelope protein optimized for infecting a cell; and at least one helper plasmid for expressing gag, pol, and rev genes, wherein when the lentiviral vector, the envelope plasmid, and the at least one helper plasmid are transfected into a packaging cell line, a lentiviral particle is produced by the packaging cell line, wherein the lentiviral particle is capable of inhibiting of producing PAH and/or inhibiting the expression of endogenous PAH.

In another aspect, the lentiviral vector, which is also referred to herein as a therapeutic vector, includes the following elements: hybrid 5′ long terminal repeat (RSV/5′ LTR) (SEQ ID NOS: 7-8), Psi sequence (RNA packaging site) (SEQ ID NO: 9), RRE (Rev-response element) (SEQ ID NO: 10), cPPT (polypurine tract) (SEQ ID NO: 11), Anti alpha trypsin promoter (hAAT) (SEQ ID NO: 12), Phenylalanine hydroxylase (PAH) (SEQ ID NO: 1-4, Woodchuck Post-Transcriptional Regulatory Element (WPRE) (SEQ ID NOS: 13), and ΔU3 3′ LTR (SEQ ID NO: 14). In embodiments, sequence variation, by way of substitution, deletion, another aspect, the lentiviral vector, which is also referred to herein as a therapeutic vector, includes the following elements: hybrid 5′ long terminal repeat (RSV/5′ LTR) (SEQ ID NOS: 7-8), Psi sequence (RNA packaging site) (SEQ ID NO: 9), RRE (Rev-response element) (SEQ ID NO: 10), cPPT (polypurine tract) (SEQ ID NO: 11), H1 promoter (SEQ ID NO: 15), PAH shRNA (SEQ ID NO: 1-4), Anti alpha trypsin promoter (hAAT) (SEQ ID NO: 12), PAH shRNA (SEQ ID NO: 1-4), Woodchuck Post-Transcriptional Regulatory Element (WPRE) (SEQ ID NOS: 13), and ΔU3 3′ LTR (SEQ ID NO: 14). In embodiments, sequence variation, by way of substitution, deletion, addition, or mutation can be used to modify the sequences references herein.

In another aspect, a helper plasmid includes the following elements: CAG promoter (SEQ ID NO: 16); HIV component gag (SEQ ID NO: 17); HIV component pol (SEQ ID NO: 18); HIV Int (SEQ ID NO: 19); HIV RRE (SEQ ID NO: 20); and HIV Rev (SEQ ID NO: 21). In another aspect, the helper plasmid may be modified to include a first helper plasmid for expressing the gag and pol genes, and a second and separate plasmid for expressing the rev gene. In embodiments, sequence variation, by way of substitution, deletion, addition, or mutation can be used to modify the sequences references herein.

In another aspect, an envelope plasmid includes the following elements: RNA polymerase II promoter (CMV) (SEQ ID NO: 22) and vesicular stomatitis virus G glycoprotein (VSV-G) (SEQ ID NO: 23). In embodiments, sequence variation, by way of substitution, deletion, addition, or mutation can be used to modify the sequences references herein.

In various aspects, the plasmids used for lentiviral packaging are modified by substitution, addition, subtraction or mutation of various elements without loss of vector function. For example, and without limitation, the following elements can replace similar elements in the plasmids that comprise the packaging system: Elongation Factor-1 (EF-1), phosphoglycerate kinase (PGK), and ubiquitin C (UbC) promoters can replace the CMV or CAG promoter. SV40 poly A and bGH poly A can replace the rabbit beta globin poly A. The HIV sequences in the helper plasmid can be constructed from different HIV strains or clades. The VSV-G glycoprotein can be substituted with membrane glycoproteins from feline endogenous virus (RD114), gibbon ape leukemia virus (GALV), Rabies (FUG), lymphocytic choriomeningitis virus (LCMV), influenza A fowl plague virus (FPV), Ross River alphavirus (RRV), murine leukemia virus 10A1 (MLV), or Ebola virus (EboV).

Various lentiviral packaging systems can be acquired commercially (e.g., Lenti-vpak packaging kit from OriGene Technologies, Inc., Rockville, MD), and can also be designed as described herein. Moreover, it is within the skill of a person ordinarily skilled in the art to substitute or modify aspects of a lentiviral packaging system to improve any number of relevant factors, including the production efficiency of a lentiviral particle.

In another aspect, adeno-associated viral (AAV) vectors can be used.

AAV Vector Construction. PAH shRNA sequence #1 (SEQ ID NO: 5) or PAH shRNA sequence #2 (SEQ ID NO: 6) can be inserted into the pAAV plasmid (Cell Biolabs). PAH oligonucleotide sequences containing BamHI and EcoRI restriction sites can be synthesized by Eurofins MWG Operon. Overlapping sense and antisense oligonucleotide sequences can be mixed and annealed during cooling from 70 degrees Celsius to room temperature. The pAAV can be digested with the restriction enzymes BamHI and EcoRI for one hour at 37 degrees Celsius. The digested pAAV plasmid can be purified by agarose gel electrophoresis and extracted from the gel using a DNA gel extraction kit from Thermo Scientific. The DNA concentrations can be determined and vector to oligo (3:1 ratio) can be mixed, allowed to anneal, and ligated. The ligation reaction can be performed with T4 DNA ligase for 30 minutes at room temperature. 2.5 microliters of the ligation mix can be added to 25 microliters of STBL3 competent bacterial cells. Transformation can be achieved after heat-shock at 42 degrees Celsius. Bacterial cells can be spread on agar plates containing ampicillin and drug-resistant colonies (indicating the presence of ampicillin-resistance plasmids) can be recovered and expanded in LB broth. To check for insertion of the oligo sequences, plasmid DNA can be extracted from harvested bacteria cultures with the Thermo Scientific DNA mini prep kit. Insertion of shRNA sequences in the pAAV plasmid can be verified by DNA sequencing using a specific primer for the promoter used to regulate shRNA expression.

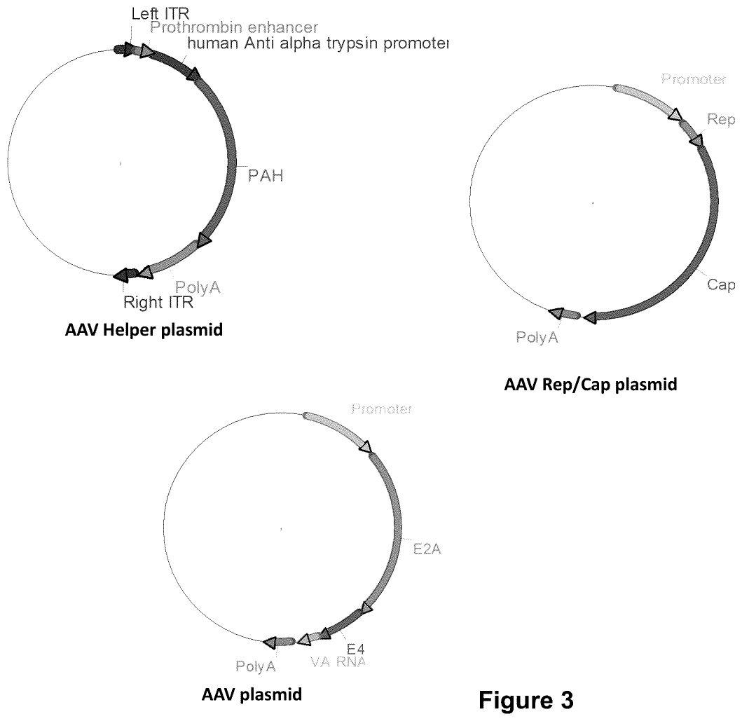

An exemplary AAV plasmid system for expressing PAH (SEQ ID NO: 1) is depicted in FIG. 3. Briefly, the leftmost AAV Helper plasmid contains a Left ITR (SEQ ID NO: 47), a Prothrombin enhancer (SEQ ID NO: 48), a human Anti alpha trypsin promoter (SEQ ID NO: 12), a PAH element (SEQ ID NO: 1), a PolyA element (SEQ ID NO: 49), and a Right ITR (SEQ ID NO: 50). The AAV plasmid depicted in the middle of FIG. 3 shows an AAV plasmid that contains a suitable promoter element (SEQ ID NO: 16; SEQ ID NO: 22), and E2A element (SEQ ID NO: 51), an E4 element (SEQ ID NO: 52), a VA RNA element (SEQ ID NO: 53), and a PolyA element (SEQ ID NO: 49). The rightmost plasmid depicts an AAV Rep/Cap plasmid that contains a suitable promoter element, a Rep element (SEQ ID NO: 54), a Cap element (SEQ ID NO: 55), and a PolyA element (SEQ ID NO: 49).

Production of AAV particles. The AAV-PAH shRNA plasmid may be combined with the plasmids pAAV-RC2 (Cell Biolabs) and pHelper (Cell Biolabs). The pAAV-RC2 plasmid may contain the Rep and AAV2 capsid genes and pHelper may contain the adenovirus E2A, E4, and VA genes. To produce AAV particles, these plasmids may be transfected in the ratio 1:1:1 (pAAV-shPAH:pAAV-RC2:pHelper) into 293T cells. For transfection of cells in 150 mm dishes (BD Falcon), 10 micrograms of each plasmid may be added together in 1 ml of DMEM. In another tube, 60 microliters of the transfection reagent PEI (1 microgram/ml) (Polysciences) may be added to 1 ml of DMEM. The two tubes may be mixed together and allowed to incubate for 15 minutes. Then the transfection mixture may be added to cells and the cells may be collected after 3 days. The cells may be lysed by freeze/thaw lysis in dry ice/isopropanol. Benzonase nuclease (Sigma) may be added to the cell lysate for 30 minutes at 37 degrees Celsius. Cell debris may then be pelleted by centrifugation at 4 degrees Celsius for 15 minutes at 12,000 rpm. The supernatant may be collected and then added to target cells.

### Dosage and Dosage Forms

The disclosed vector compositions allow for short, medium, or long-term expression of genes or sequences of interest and episomal maintenance of the disclosed vectors. Accordingly, dosing regimens may vary based upon the condition being treated and the method of administration.

In embodiments, vector compositions may be administered to a subject in need in varying doses. Specifically, a subject may be administered about ≥10⁶ infectious doses (where 1 dose is needed on average to transduce 1 target cell). More specifically, a subject may be administered about ≥10⁷, about ≥10⁸, about ≥10⁹, or about ≥10¹⁰ infectious doses, or any number of doses in-between these values. Upper limits of dosing will be determined for each disease indication, including a specific cancer type, and will depend on toxicity/safety profiles for each individual product or product lot.

Additionally, vector compositions of the present disclosure may be administered periodically, such as once or twice a day, or any other suitable time period. For example, vector compositions may be administered to a subject in need once a week, once every other week, once every three weeks, once a month, every other month, every three months, every six months, every nine months, once a year, every eighteen months, every two years, every thirty months, or every three years.

In embodiments, the disclosed vector compositions are administered as a pharmaceutical composition. In embodiments, the pharmaceutical composition can be formulated in a wide variety of dosage forms, including but not limited to nasal, pulmonary, oral, topical, or parenteral dosage forms for clinical application. Each of the dosage forms can comprise various solubilizing agents, disintegrating agents, surfactants, fillers, thickeners, binders, diluents such as wetting agents or other pharmaceutically acceptable excipients. The pharmaceutical composition can also be formulated for injection, insufflation, infusion, or intradermal exposure. For instance, an injectable formulation may comprise the disclosed vectors in an aqueous or non-aqueous solution at a suitable pH and tonicity.

The disclosed vector compositions may be administered to a subject via direct injection into a tumor site or at a site of infection. In some embodiments, the vectors can be administered systemically. In some embodiments, the vector compositions can be administered via guided cannulation to tissues immediately surrounding the sites of tumor or infection.

The disclosed vector compositions can be administered using any pharmaceutically acceptable method, such as intranasal, buccal, sublingual, oral, rectal, ocular, parenteral (intravenously, intradermally, intramuscularly, subcutaneously, intraperitoneally), pulmonary, intravaginal, locally administered, topically administered, topically administered after scarification, mucosally administered, via an aerosol, in semi-solid media such as agarose or gelatin, or via a buccal or nasal spray formulation.

Further, the disclosed vector compositions can be formulated into any pharmaceutically acceptable dosage form, such as a solid dosage form, tablet, pill, lozenge, capsule, liquid dispersion, gel, aerosol, pulmonary aerosol, nasal aerosol, ointment, cream, semi-solid dosage form, a solution, an emulsion, and a suspension. Further, the pharmaceutical composition may be a controlled release formulation, sustained release formulation, immediate release formulation, or any combination thereof. Further, the pharmaceutical composition may be a transdermal delivery system.

In embodiments, the pharmaceutical composition can be formulated in a solid dosage form for oral administration, and the solid dosage form can be powders, granules, capsules, tablets or pills. In embodiments, the solid dosage form can include one or more excipients such as calcium carbonate, starch, sucrose, lactose, microcrystalline cellulose or gelatin. In addition, the solid dosage form can include, in addition to the excipients, a lubricant such as talc or magnesium stearate. In some embodiments, the oral dosage form can be immediate release, or a modified release form. Modified release dosage forms include controlled or extended release, enteric release, and the like. The excipients used in the modified release dosage forms are commonly known to a person of ordinary skill in the art.

In embodiments, the pharmaceutical compositions can be formulated as sublingual or buccal dosage forms. Such dosage forms comprise sublingual tablets or solution compositions that are administered under the tongue and buccal tablets that are placed between the cheek and gum.

In embodiments, the pharmaceutical compositions can be formulated as nasal dosage forms. Such dosage forms of the present invention comprise solution, suspension, and gel compositions for nasal delivery.

In embodiments, the pharmaceutical compositions can be formulated in liquid dosage forms for oral administration, such as suspensions, emulsions or syrups. In embodiments, the liquid dosage forms can include, in addition to commonly used simple diluents such as water and liquid paraffin, various excipients such as humectants, sweeteners, aromatics or preservatives. In embodiments, the compositions can be formulated to be suitable for administration to a pediatric patient.

In embodiments, the pharmaceutical compositions can be formulated in dosage forms for parenteral administration, such as sterile aqueous solutions, suspensions, emulsions, non-aqueous solutions or suppositories. In embodiments, the solutions or suspensions can include propylene glycol, polyethylene glycol, vegetable oils such as olive oil or injectable esters such as ethyl oleate.

The dosage of the pharmaceutical compositions can vary depending on the patient's weight, age, gender, administration time and mode, excretion rate, and the severity of disease.

In embodiments, the vector compositions are administered into the cerebrospinal fluid, blood or lymphatic circulation by venous or arterial cannulation or injection, intradermal delivery, intramuscular delivery or injection into a draining organ near the site of disease.

The following examples are given to illustrate aspects of the present invention. It should be understood, however, that the invention is not to be limited to the specific conditions or details described in these examples. All printed publications referenced herein are specifically incorporated by reference.

### EXAMPLES

### Example 1: Development of a Lentiviral Vector System

A lentiviral vector system was developed as summarized in FIG. 1 (circularized form). Lentiviral particles were produced in 293T/17 HEK cells (purchased from American Type Culture Collection, Manassas, VA) following transfection with the therapeutic vector, the envelope plasmid, and the helper plasmid. The transfection of 293T/17 HEK cells, which produced functional viral particles, employed the reagent Poly(ethylenimine) (PEI) to increase the efficiency of plasmid DNA uptake. The plasmids and DNA were initially added separately in culture medium without serum in a ratio of 3:1 (mass ratio of PEI to DNA). After 2-3 days, cell medium was collected and lentiviral particles were purified by high-speed centrifugation and/or filtration followed by anion-exchange chromatography. The concentration of lentiviral particles can be expressed in terms of transducing units/ml (TU/ml). The determination of TU was accomplished by measuring HIV p24 levels in culture fluids (p24 protein is incorporated into lentiviral particles), by measuring the number of viral DNA copies per transduced cell by quantitative PCR, or by infecting cells and using light (if the vectors encode luciferase or fluorescent protein markers).

As mentioned above, a 3-vector system (i.e., which includes a 2-vector lentiviral packaging system) was designed for the production of lentiviral particles. A schematic of the 3-vector system is shown in FIG. 1. Briefly, and with reference to FIG. 1, the top-most vector is a helper plasmid, which, in this case, includes Rev. The vector appearing in the middle of FIG. 1 is the envelope plasmid. The bottom-most vector is the therapeutic vector, as described herein.

Referring to FIG. 1, the Helper plus Rev plasmid includes a CAG enhancer (SEQ ID NO: 24); a CAG promoter (SEQ ID NO: 16); a chicken beta actin intron (SEQ ID NO: 25); a HIV gag (SEQ ID NO: 17); a HIV Pol (SEQ ID NO: 18); a HIV Int (SEQ ID NO: 19); a HIV RRE (SEQ ID NO: 20); a HIV Rev (SEQ ID NO: 21); and a rabbit beta globin poly A (SEQ ID NO: 26).

The Envelope plasmid includes a CMV promoter (SEQ ID NO: 22); a beta globin intron (SEQ ID NO: 27); a VSV-G envelope glycoprotein (SEQ ID NO: 23); and a rabbit beta globin poly A (SEQ ID NO: 26).

Synthesis of a 3-vector system, which includes a 2-vector lentiviral packaging system, consisting of Helper (plus Rev) and Envelope plasmids, is disclosed.

### Materials and Methods:

Construction of the helper plasmid: The helper plasmid was constructed by initial PCR amplification of a DNA fragment from the pNL4-3 HIV plasmid (NIH Aids Reagent Program) containing Gag, Pol, and Integrase genes. Primers were designed to amplify the fragment with EcoRI and NotI restriction sites which could be used to insert at the same sites in the pCDNA3 plasmid (Invitrogen). The forward primer was (5′-TAAGCAGAATTCATGAATTTGCCAGGAAGAT-3′) (SEQ ID NO: 28) and reverse primer was (5′-CCATACAATGAATGGACACTAGGCGGCCGCACGAAT-3′) (SEQ ID NO: 29).

The sequence for the Gag, Pol, Integrase fragment was as follows:

(SEQ ID NO: 30)
GAATTCATGAATTTGCCAGGAAGATGGAAACCAAAAATGATAGGGGGAAT
TGGAGGTTTTATCAAAGTAAGACAGTATGATCAGATACTCATAGAAATCT
GCGGACATAAAGCTATAGGTACAGTATTAGTAGGACCTACACCTGTCAAC
ATAATTGGAAGAAATCTGTTGACTCAGATTGGCTGCACTTTAAATTTTCC
CATTAGTCCTATTGAGACTGTACCAGTAAAATTAAAGCCAGGAATGGATG
GCCCAAAAGTTAAACAATGGCCATTGACAGAAGAAAAAATAAAAGCATTA
GTAGAAATTTGTACAGAAATGGAAAAGGAAGGAAAAATTTCAAAAATTGG
GCCTGAAAATCCATACAATACTCCAGTATTTGCCATAAAGAAAAAAGACA
GTACTAAATGGAGAAAATTAGTAGATTTCAGAGAACTTAATAAGAGAACT
CAAGATTTCTGGGAAGTTCAATTAGGAATACCACATCCTGCAGGGTTAAA
ACAGAAAAAATCAGTAACAGTACTGGATGTGGGCGATGCATATTTTTCAG
TTCCCTTAGATAAAGACTTCAGGAAGTATACTGCATTTACCATACCTAGT
ATAAACAATGAGACACCAGGGATTAGATATCAGTACAATGTGCTTCCACA
GGGATGGAAAGGATCACCAGCAATATTCCAGTGTAGCATGACAAAAATCT
TAGAGCCTTTTAGAAAACAAAATCCAGACATAGTCATCTATCAATACATG
GATGATTTGTATGTAGGATCTGACTTAGAAATAGGGCAGCATAGAACAAA
AATAGAGGAACTGAGACAACATCTGTTGAGGTGGGGATTTACCACACCAG
ACAAAAAACATCAGAAAGAACCTCCATTCCTTTGGATGGGTTATGAACTC
CATCCTGATAAATGGACAGTACAGCCTATAGTGCTGCCAGAAAAGGACAG
CTGGACTGTCAATGACATACAGAAATTAGTGGGAAAATTGAATTGGGCAA
GTCAGATTTATGCAGGGATTAAAGTAAGGCAATTATGTAAACTTCTTAGG
GGAACCAAAGCACTAACAGAAGTAGTACCACTAACAGAAGAAGCAGAGCT
AGAACTGGCAGAAAACAGGGAGATTCTAAAAGAACCGGTACATGGAGTGT
ATTATGACCCATCAAAAGACTTAATAGCAGAAATACAGAAGCAGGGGCAA
GGCCAATGGACATATCAAATTTATCAAGAGCCATTTAAAAATCTGAAAAC
AGGAAAGTATGCAAGAATGAAGGGTGCCCACACTAATGATGTGAAACAAT
TAACAGAGGCAGTACAAAAAATAGCCACAGAAAGCATAGTAATATGGGGA
AAGACTCCTAAATTTAAATTACCCATACAAAAGGAAACATGGGAAGCATG
GTGGACAGAGTATTGGCAAGCCACCTGGATTCCTGAGTGGGAGTTTGTCA
ATACCCCTCCCTTAGTGAAGTTATGGTACCAGTTAGAGAAAGAACCCATA
ATAGGAGCAGAAACTTTCTATGTAGATGGGGCAGCCAATAGGGAAACTAA
ATTAGGAAAAGCAGGATATGTAACTGACAGAGGAAGACAAAAAGTTGTCC
CCCTAACGGACACAACAAATCAGAAGACTGAGTTACAAGCAATTCATCTA
GCTTTGCAGGATTCGGGATTAGAAGTAAACATAGTGACAGACTCACAATA
TGCATTGGGAATCATTCAAGCACAACCAGATAAGAGTGAATCAGAGTTAG
TCAGTCAAATAATAGAGCAGTTAATAAAAAAGGAAAAAGTCTACCTGGCA
TGGGTACCAGCACACAAAGGAATTGGAGGAAATGAACAAGTAGATAAATT
GGTCAGTGCTGGAATCAGGAAAGTACTATTTTTAGATGGAATAGATAAGG
CCCAAGAAGAACATGAGAAATATCACAGTAATTGGAGAGCAATGGCTAGT
GATTTTAACCTACCACCTGTAGTAGCAAAAGAAATAGTAGCCAGCTGTGA
TAAATGTCAGCTAAAAGGGGAAGCCATGCATGGACAAGTAGACTGTAGCC
CAGGAATATGGCAGCTAGATTGTACACATTTAGAAGGAAAAGTTATCTTG
GTAGCAGTTCATGTAGCCAGTGGATATATAGAAGCAGAAGTAATTCCAGC
AGAGACAGGGCAAGAAACAGCATACTTCCTCTTAAAATTAGCAGGAAGAT
GGCCAGTAAAAACAGTACATACAGACAATGGCAGCAATTTCACCAGTACT
ACAGTTAAGGCCGCCTGTTGGTGGGCGGGGATCAAGCAGGAATTTGGCAT
TCCCTACAATCCCCAAAGTCAAGGAGTAATAGAATCTATGAATAAAGAAT
TAAAGAAAATTATAGGACAGGTAAGAGATCAGGCTGAACATCTTAAGACA
GCAGTACAAATGGCAGTATTCATCCACAATTTTAAAAGAAAAGGGGGGAT
TGGGGGGTACAGTGCAGGGGAAAGAATAGTAGACATAATAGCAACAGACA
TACAAACTAAAGAATTACAAAAACAAATTACAAAAATTCAAAATTTTCGG
GTTTATTACAGGGACAGCAGAGATCCAGTTTGGAAAGGACCAGCAAAGCT
CCTCTGGAAAGGTGAAGGGGCAGTAGTAATACAAGATAATAGTGACATAA
AAGTAGTGCCAAGAAGAAAAGCAAAGATCATCAGGGATTATGGAAAACAG
ATGGCAGGTGATGATTGTGTGGCAAGTAGACAGGATGAGGATTAA

Next, a DNA fragment containing the Rev, RRE, and rabbit beta globin poly A sequence with XbaI and XmaI flanking restriction sites was synthesized by Eurofins Genomics. The DNA fragment was then inserted into the plasmid at the XbaI and XmaI restriction sites The DNA sequence was as follows:

(SEQ ID NO: 31)
TCTAGAATGGCAGGAAGAAGCGGAGACAGCGACGAAGAGCTCATCAGAAC
AGTCAGACTCATCAAGCTTCTCTATCAAAGCAACCCACCTCCCAATCCCG
AGGGGACCCGACAGGCCCGAAGGAATAGAAGAAGAAGGTGGAGAGAGAGA
CAGAGACAGATCCATTCGATTAGTGAACGGATCCTTGGCACTTATCTGGG
ACGATCTGCGGAGCCTGTGCCTCTTCAGCTACCACCGCTTGAGAGACTTA
CTCTTGATTGTAACGAGGATTGTGGAACTTCTGGGACGCAGGGGGTGGGA
AGCCCTCAAATATTGGTGGAATCTCCTACAATATTGGAGTCAGGAGCTAA
AGAATAGAGGAGCTTTGTTCCTTGGGTTCTTGGGAGCAGCAGGAAGCACT
ATGGGCGCAGCGTCAATGACGCTGACGGTACAGGCCAGACAATTATTGTC
TGGTATAGTGCAGCAGCAGAACAATTTGCTGAGGGCTATTGAGGCGCAAC
AGCATCTGTTGCAACTCACAGTCTGGGGCATCAAGCAGCTCCAGGCAAGA
ATCCTGGCTGTGGAAAGATACCTAAAGGATCAACAGCTCCTAGATCTTTT
TCCCTCTGCCAAAAATTATGGGGACATCATGAAGCCCCTTGAGCATCTGA
CTTCTGGCTAATAAAGGAAATTTATTTTCATTGCAATAGTGTGTTGGAAT
TTTTTGTGTCTCTCACTCGGAAGGACATATGGGAGGGCAAATCATTTAAA
ACATCAGAATGAGTATTTGGTTTAGAGTTTGGCAACATATGCCATATGCT
GGCTGCCATGAACAAAGGTGGCTATAAAGAGGTCATCAGTATATGAAACA
GCCCCCTGCTGTCCATTCCTTATTCCATAGAAAAGCCTTGACTTGAGGTT
AGATTTTTTTTATATTTTGTTTTGTGTTATTTTTTTCTTTAACATCCCTA
AAATTTTCCTTACATGTTTTACTAGCCAGATTTTTCCTCCTCTCCTGACT
ACTCCCAGTCATAGCTGTCCCTCTTCTCTTATGAAGATCCCTCGACCTGC
AGCCCAAGCTTGGCGTAATCATGGTCATAGCTGTTTCCTGTGTGAAATTG
TTATCCGCTCACAATTCCACACAACATACGAGCCGGAAGCATAAAGTGTA
AAGCCTGGGGTGCCTAATGAGTGAGCTAACTCACATTAATTGCGTTGCGC
TCACTGCCCGCTTTCCAGTCGGGAAACCTGTCGTGCCAGCGGATCCGCAT
CTCAATTAGTCAGCAACCATAGTCCCGCCCCTAACTCCGCCCATCCCGCC
CCTAACTCCGCCCAGTTCCGCCCATTCTCCGCCCCATGGCTGACTAATTT
TTTTTATTTATGCAGAGGCCGAGGCCGCCTCGGCCTCTGAGCTATTCCAG
AAGTAGTGAGGAGGCTTTTTTGGAGGCCTAGGCTTTTGCAAAAAGCTAAC
TTGTTTATTGCAGCTTATAATGGTTACAAATAAAGCAATAGCATCACAAA
TTTCACAAATAAAGCATTTTTTTCACTGCATTCTAGTTGTGGTTTGTCCA
AACTCATCAATGTATCTTATCAGCGGCCGCCCCGGG

Finally, the CMV promoter of pCDNA3.1 was replaced with the CAG enhancer/promoter plus a chicken beta actin intron sequence. A DNA fragment containing the CAG enhancer/promoter/intron sequence with MluI and EcoRI flanking restriction sites was synthesized by Eurofins Genomics. The DNA fragment was then inserted into the plasmid at the MluI and EcoRI restriction sites. The DNA sequence was as follows:

(SEQ ID NO: 32)
ACGCGTTAGTTATTAATAGTAATCAATTACGGGGTCATTAGTTCATAGCC
CATATATGGAGTTCCGCGTTACATAACTTACGGTAAATGGCCCGCCTGGC
TGACCGCCCAACGACCCCCGCCCATTGACGTCAATAATGACGTATGTTCC
CATAGTAACGCCAATAGGGACTTTCCATTGACGTCAATGGGTGGACTATT
TACGGTAAACTGCCCACTTGGCAGTACATCAAGTGTATCATATGCCAAGT
ACGCCCCCTATTGACGTCAATGACGGTAAATGGCCCGCCTGGCATTATGC
CCAGTACATGACCTTATGGGACTTTCCTACTTGGCAGTACATCTACGTAT
TAGTCATCGCTATTACCATGGGTCGAGGTGAGCCCCACGTTCTGCTTCAC
TCTCCCCATCTCCCCCCCCTCCCCACCCCCAATTTTGTATTTATTTATTT
TTTAATTATTTTGTGCAGCGATGGGGGCGGGGGGGGGGGGGGCGCGCGCC
AGGCGGGGCGGGGCGGGGCGAGGGGCGGGGCGGGGCGAGGCGGAGAGGTG
CGGCGGCAGCCAATCAGAGCGGCGCGCTCCGAAAGTTTCCTTTTATGGCG
AGGCGGCGGCGGCGGCGGCCCTATAAAAAGCGAAGCGCGCGGCGGGCGGG
AGTCGCTGCGTTGCCTTCGCCCCGTGCCCCGCTCCGCGCCGCCTCGCGCC
GCCCGCCCCGGCTCTGACTGACCGCGTTACTCCCACAGGTGAGCGGGCGG
GACGGCCCTTCTCCTCCGGGCTGTAATTAGCGCTTGGTTTAATGACGGCT
CGTTTCTTTTCTGTGGCTGCGTGAAAGCCTTAAAGGGCTCCGGGAGGGCC
CTTTGTGCGGGGGGGAGCGGCTCGGGGGGTGCGTGCGTGTGTGTGTGCGT
GGGGAGCGCCGCGTGCGGCCCGCGCTGCCCGGCGGCTGTGAGCGCTGCGG
GCGCGGCGCGGGGCTTTGTGCGCTCCGCGTGTGCGCGAGGGGAGCGCGGC
CGGGGGCGGTGCCCCGCGGTGCGGGGGGGCTGCGAGGGGAACAAAGGCTG
CGTGCGGGGTGTGTGCGTGGGGGGGTGAGCAGGGGGTGTGGGCGCGGCGG
TCGGGCTGTAACCCCCCCCTGCACCCCCCTCCCCGAGTTGCTGAGCACGG
CCCGGCTTCGGGTGCGGGGCTCCGTGCGGGGCGTGGCGCGGGGCTCGCCG
TGCCGGGCGGGGGGTGGCGGCAGGTGGGGGTGCCGGGCGGGGCGGGGCCG
CCTCGGGCCGGGGAGGGCTCGGGGGAGGGGCGCGGCGGCCCCGGAGCGCC
GGCGGCTGTCGAGGCGCGGCGAGCCGCAGCCATTGCCTTTTATGGTAATC
GTGCGAGAGGGCGCAGGGACTTCCTTTGTCCCAAATCTGGCGGAGCCGAA
ATCTGGGAGGCGCCGCCGCACCCCCTCTAGCGGGCGCGGGCGAAGCGGTG
CGGCGCCGGCAGGAAGGAAATGGGCGGGGAGGGCCTTCGTGCGTCGCCGC
GCCGCCGTCCCCTTCTCCATCTCCAGCCTCGGGGCTGCCGCAGGGGGACG
GCTGCCTTCGGGGGGGACGGGGCAGGGCGGGGTTCGGCTTCTGGCGTGTG
ACCGGCGGGAATTC

Construction of the VSV-G Envelope Plasmid:

The vesicular stomatitis Indiana virus glycoprotein (VSV-G) sequence was synthesized by Eurofins Genomics with flanking EcoRI restriction sites. The DNA fragment was then inserted into the pCDNA3.1 plasmid (Invitrogen) at the EcoRI restriction site and the correct orientation was determined by sequencing using a CMV specific primer.

The DNA sequence was as follows:

(SEQ ID NO: 23)
GAATTCATGAAGTGCCTTTTGTACTTAGCCTTTTTATTCATTGGGGTGAA
TTGCAAGTTCACCATAGTTTTTCCACACAACCAAAAAGGAAACTGGAAAA
ATGTTCCTTCTAATTACCATTATTGCCCGTCAAGCTCAGATTTAAATTGG
CATAATGACTTAATAGGCACAGCCTTACAAGTCAAAATGCCCAAGAGTCA
CAAGGCTATTCAAGCAGACGGTTGGATGTGTCATGCTTCCAAATGGGTCA
CTACTTGTGATTTCCGCTGGTATGGACCGAAGTATATAACACATTCCATC
CGATCCTTCACTCCATCTGTAGAACAATGCAAGGAAAGCATTGAACAAAC
GAAACAAGGAACTTGGCTGAATCCAGGCTTCCCTCCTCAAAGTTGTGGAT
ATGCAACTGTGACGGATGCCGAAGCAGTGATTGTCCAGGTGACTCCTCAC
CATGTGCTGGTTGATGAATACACAGGAGAATGGGTTGATTCACAGTTCAT
CAACGGAAAATGCAGCAATTACATATGCCCCACTGTCCATAACTCTACAA
CCTGGCATTCTGACTATAAGGTCAAAGGGCTATGTGATTCTAACCTCATT
TCCATGGACATCACCTTCTTCTCAGAGGACGGAGAGCTATCATCCCTGGG
AAAGGAGGGCACAGGGTTCAGAAGTAACTACTTTGCTTATGAAACTGGAG
GCAAGGCCTGCAAAATGCAATACTGCAAGCATTGGGGAGTCAGACTCCCA
TCAGGTGTCTGGTTCGAGATGGCTGATAAGGATCTCTTTGCTGCAGCCAG
ATTCCCTGAATGCCCAGAAGGGTCAAGTATCTCTGCTCCATCTCAGACCT
CAGTGGATGTAAGTCTAATTCAGGACGTTGAGAGGATCTTGGATTATTCC
CTCTGCCAAGAAACCTGGAGCAAAATCAGAGCGGGTCTTCCAATCTCTCC
AGTGGATCTCAGCTATCTTGCTCCTAAAAACCCAGGAACCGGTCCTGCTT
TCACCATAATCAATGGTACCCTAAAATACTTTGAGACCAGATACATCAGA
GTCGATATTGCTGCTCCAATCCTCTCAAGAATGGTCGGAATGATCAGTGG
AACTACCACAGAAAGGGAACTGTGGGATGACTGGGCACCATATGAAGACG
TGGAAATTGGACCCAATGGAGTTCTGAGGACCAGTTCAGGATATAAGTTT
CCTTTATACATGATTGGACATGGTATGTTGGACTCCGATCTTCATCTTAG
CTCAAAGGCTCAGGTGTTCGAACATCCTCACATTCAAGACGCTGCTTCGC
AACTTCCTGATGATGAGAGTTTATTTTTTGGTGATACTGGGCTATCCAAA
AATCCAATCGAGCTTGTAGAAGGTTGGTTCAGTAGTTGGAAAAGCTCTAT
TGCCTCTTTTTTCTTTATCATAGGGTTAATCATTGGACTATTCTTGGTTC
TCCGAGTTGGTATCCATCTTTGCATTAAATTAAAGCACACCAAGAAAAGA
CAGATTTATACAGACATAGAGATGAGAATTC

A 4-vector system, which includes a 3-vector lentiviral packaging system, has also been designed and produced using the methods and materials described herein. A schematic of the 4-vector system is shown in FIG. 2. Briefly, and with reference to FIG. 2, the top-most vector is a helper plasmid, which, in this case, does not include Rev. The vector second from the top is a separate Rev plasmid. The vector second from the bottom is the envelope plasmid. The bottom-most vector is the therapeutic vector as described herein.

Referring to FIG. 2, the Helper plasmid includes a CAG enhancer (SEQ ID NO: 24); a CAG promoter (SEQ ID NO: 16); a chicken beta actin intron (SEQ ID NO: 25); a HIV gag (SEQ ID NO: 17); a HIV Pol (SEQ ID NO: 18); a HIV Int (SEQ ID NO: 19); a HIV RRE (SEQ ID NO: 20); and a rabbit beta globin poly A (SEQ ID NO: 26).

The Rev plasmid includes a RSV promoter (SEQ ID NO: 7); a HIV Rev (SEQ ID NO: 21); and a rabbit beta globin poly A (SEQ ID NO: 26).

The Envelope plasmid includes a CMV promoter (SEQ ID NO: 22); a beta globin intron (SEQ ID NO: 27); a VSV-G (SEQ ID NO: 23); and a rabbit beta globin poly A (SEQ ID NO: 26).

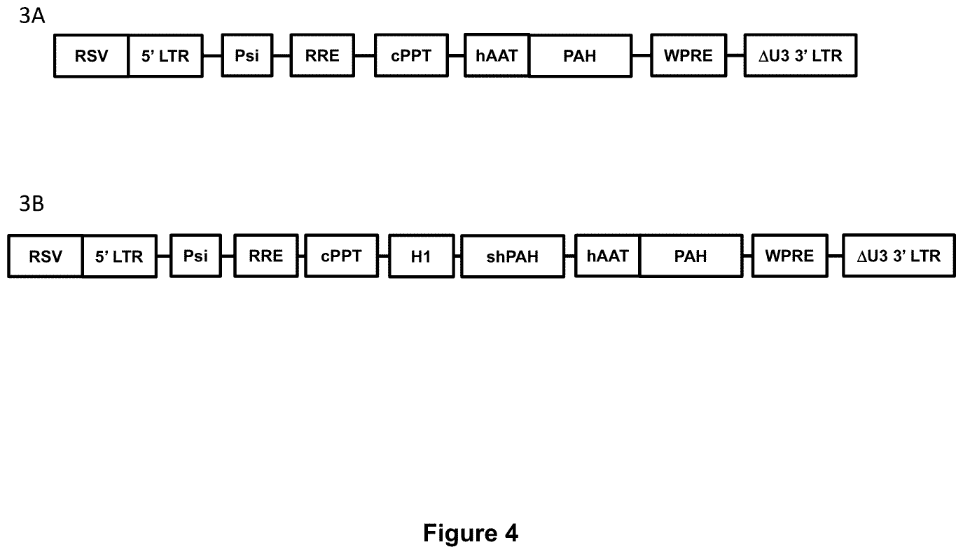

In one aspect, the therapeutic PAH lentivirus plasmid includes all of the elements shown in FIG. 4A. In another aspect, the therapeutic PAH lentivirus plasmid includes all of the elements shown in FIG. 4B.

Synthesis of a 4-vector system, which includes a 3-vector lentiviral packaging system consisting of Helper, Rev, and Envelope plasmids, is disclosed.

### Materials and Methods:

Construction of the Helper Plasmid without Rev:

The Helper plasmid without Rev was constructed by inserting a DNA fragment containing the RRE and rabbit beta globin poly A sequence. This sequence was synthesized by Eurofins Genomics with flanking XbaI and XmaI restriction sites. The RRE/rabbit poly A beta globin sequence was then inserted into the Helper plasmid at the XbaI and XmaI restriction sites.

The DNA sequence is as follows:

(SEQ ID NO: 56)
TCTAGAAGGAGCTTTGTTCCTTGGGTTCTTGGGAGCAGCAGGAAGCACTA
TGGGCGCAGCGTCAATGACGCTGACGGTACAGGCCAGACAATTATTGTCT
GGTATAGTGCAGCAGCAGAACAATTTGCTGAGGGCTATTGAGGCGCAACA
GCATCTGTTGCAACTCACAGTCTGGGGCATCAAGCAGCTCCAGGCAAGAA
TCCTGGCTGTGGAAAGATACCTAAAGGATCAACAGCTCCTAGATCTTTTT
CCCTCTGCCAAAAATTATGGGGACATCATGAAGCCCCTTGAGCATCTGAC
TTCTGGCTAATAAAGGAAATTTATTTTCATTGCAATAGTGTGTTGGAATT
TTTTGTGTCTCTCACTCGGAAGGACATATGGGAGGGCAAATCATTTAAAA
CATCAGAATGAGTATTTGGTTTAGAGTTTGGCAACATATGCCATATGCTG
GCTGCCATGAACAAAGGTGGCTATAAAGAGGTCATCAGTATATGAAACAG
CCCCCTGCTGTCCATTCCTTATTCCATAGAAAAGCCTTGACTTGAGGTTA
GATTTTTTTTATATTTTGTTTTGTGTTATTTTTTTCTTTAACATCCCTAA
AATTTTCCTTACATGTTTTACTAGCCAGATTTTTCCTCCTCTCCTGACTA
CTCCCAGTCATAGCTGTCCCTCTTCTCTTATGAAGATCCCTCGACCTGCA
GCCCAAGCTTGGCGTAATCATGGTCATAGCTGTTTCCTGTGTGAAATTGT
TATCCGCTCACAATTCCACACAACATACGAGCCGGAAGCATAAAGTGTAA
AGCCTGGGGTGCCTAATGAGTGAGCTAACTCACATTAATTGCGTTGCGCT
CACTGCCCGCTTTCCAGTCGGGAAACCTGTCGTGCCAGCGGATCCGCATC
TCAATTAGTCAGCAACCATAGTCCCGCCCCTAACTCCGCCCATCCCGCCC
CTAACTCCGCCCAGTTCCGCCCATTCTCCGCCCCATGGCTGACTAATTTT
TTTTATTTATGCAGAGGCCGAGGCCGCCTCGGCCTCTGAGCTATTCCAGA
AGTAGTGAGGAGGCTTTTTTGGAGGCCTAGGCTTTTGCAAAAAGCTAACT
TGTTTATTGCAGCTTATAATGGTTACAAATAAAGCAATAGCATCACAAAT
TTCACAAATAAAGCATTTTTTTCACTGCATTCTAGTTGTGGTTTGTCCAA
ACTCATCAATGTATCTTATCACCCGGG

Construction of the Rev Plasmid:

The RSV promoter and HIV Rev sequences were synthesized as a single DNA fragment by Eurofins Genomics with flanking MfeI and XbaI restriction sites. The DNA fragment was then inserted into the pCDNA3.1 plasmid (Invitrogen) at the MfeI and XbaI restriction sites in which the CMV promoter is replaced with the RSV promoter. The DNA sequence was as follows:

(SEQ ID NO: 33)
CAATTGCGATGTACGGGCCAGATATACGCGTATCTGAGGGGACTAGGGTG
TGTTTAGGCGAAAAGCGGGGCTTCGGTTGTACGCGGTTAGGAGTCCCCTC
AGGATATAGTAGTTTCGCTTTTGCATAGGGAGGGGGAAATGTAGTCTTAT
GCAATACACTTGTAGTCTTGCAACATGGTAACGATGAGTTAGCAACATGC
CTTACAAGGAGAGAAAAAGCACCGTGCATGCCGATTGGTGGAAGTAAGGT
GGTACGATCGTGCCTTATTAGGAAGGCAACAGACAGGTCTGACATGGATT
GGACGAACCACTGAATTCCGCATTGCAGAGATAATTGTATTTAAGTGCCT
AGCTCGATACAATAAACGCCATTTGACCATTCACCACATTGGTGTGCACC
TCCAAGCTCGAGCTCGTTTAGTGAACCGTCAGATCGCCTGGAGACGCCAT
CCACGCTGTTTTGACCTCCATAGAAGACACCGGGACCGATCCAGCCTCCC
CTCGAAGCTAGCGATTAGGCATCTCCTATGGCAGGAAGAAGCGGAGACAG
CGACGAAGAACTCCTCAAGGCAGTCAGACTCATCAAGTTTCTCTATCAAA
GCAACCCACCTCCCAATCCCGAGGGGACCCGACAGGCCCGAAGGAATAGA
AGAAGAAGGTGGAGAGAGAGACAGAGACAGATCCATTCGATTAGTGAACG
GATCCTTAGCACTTATCTGGGACGATCTGCGGAGCCTGTGCCTCTTCAGC
TACCACCGCTTGAGAGACTTACTCTTGATTGTAACGAGGATTGTGGAACT
TCTGGGACGCAGGGGGTGGGAAGCCCTCAAATATTGGTGGAATCTCCTAC
AATATTGGAGTCAGGAGCTAAAGAATAGTCTAGA

The plasmids used in the packaging systems can be modified with similar elements, and the intron sequences can potentially be removed without loss of vector function. For example, the following elements can replace similar elements in the packaging system:

Promoters: Elongation Factor-1 (EF-1) (SEQ ID NO: 34), phosphoglycerate kinase (PGK) (SEQ ID NO: 35), and ubiquitin C (UbC) (SEQ ID NO: 36) can replace the CMV (SEQ ID NO: 22) or CAG promoter (SEQ ID NO: 16). These sequences can also be further varied by addition, substitution, deletion or mutation.

Poly A sequences: SV40 poly A (SEQ ID NO: 37) and bGH poly A (SEQ ID NO: 38) can replace the rabbit beta globin poly A (SEQ ID NO: 26). These sequences can also be further varied by addition, substitution, deletion or mutation.

HIV Gag, Pol, and Integrase sequences: The HIV sequences in the Helper plasmid can be constructed from different HIV strains or clades. For example, HIV Gag (SEQ ID NO: 17); HIV Pol (SEQ ID NO: 18); and HIV Int (SEQ ID NO: 19) from the Bal strain can be interchanged with the gag, pol, and int sequences contained in the helper/helper plus Rev plasmids as outlined herein. These sequences can also be further varied by addition, substitution, deletion or mutation.

Envelope: The VSV-G glycoprotein can be substituted with membrane glycoproteins from feline endogenous virus (RD114) (SEQ ID NO: 39), gibbon ape leukemia virus (GALV) (SEQ ID NO: 40), Rabies (FUG) (SEQ ID NO: 41), lymphocytic choriomeningitis virus (LCMV) (SEQ ID NO: 42), influenza A fowl plague virus (FPV) (SEQ ID NO: 43), Ross River alphavirus (RRV) (SEQ ID NO: 44), murine leukemia virus 10A1 (MLV) (SEQ ID NO: 45), or Ebola virus (EboV) (SEQ ID NO: 46). Sequences for these envelopes are identified in the sequence portion herein. Further, these sequences can also be further varied by addition, substitution, deletion or mutation.

In summary, the 3-vector versus 4-vector systems can be compared and contrasted as follows. The 3-vector lentiviral vector system contains: 1. Helper plasmid: HIV Gag, Pol, Integrase, and Rev/Tat; 2. Envelope plasmid: VSV-G/FUG envelope; and 3. Therapeutic vector: RSV 5′LTR, Psi Packaging Signal, RRE, cPPT, ApoE Enhancer, anti-alpha trypsin promoter, phenylalanine hydroxylase, 3′ UTR, WPRE, and 3′delta LTR. The 4-vector lentiviral vector system contains: 1. Helper plasmid: HIV Gag, Pol, and Integrase; 2. Rev plasmid: Rev; 3. Envelope plasmid: VSV-G/FUG envelope; and 4. Therapeutic vector: RSV 5′LTR, Psi Packaging Signal, RRE, cPPT, ApoE Enhancer, anti-alpha trypsin promoter, phenylalanine hydroxylase, WPRE, and 3′delta LTR. Sequences corresponding with the above elements are identified in the sequence listings portion herein.

### Example 2. Therapeutic Vectors

Exemplary therapeutic vectors have been designed and developed as shown, for example, in FIG. 4.

Referring first to FIG. 4A, from left to right, the key genetic elements are as follows: hybrid 5′ long terminal repeat (RSV/LTR), Psi sequence (RNA packaging site), RRE (Rev-response element), cPPT (polypurine tract), a hAAT promoter, a PAH or variant thereof, as detailed herein, Woodchuck Post-Transcriptional Regulatory Element (WPRE), and LTR with a deletion in the U3 region.

Referring next to FIG. 4B, from left to right, the key genetic elements are as follows: hybrid 5′ long terminal repeat (RSV/LTR), Psi sequence (RNA packaging site), RRE (Rev-response element), cPPT (polypurine tract), an H1 promoter, a PAH shRNA sequence or variant thereof, as detailed herein, a hAAT promoter, a PAH sequence including the PAH sequences and variants thereof, as detailed herein, a Woodchuck Post-Transcriptional Regulatory Element (WPRE), and LTR with a deletion in the U3 region.

To produce the vectors outlined generally in FIGS. 4A and 4B, the following methods and materials were employed.

Inhibitory RNA Design: The sequence of Homo sapiens phenylalanine hydroxylase (PAH) (NM_000277.1) mRNA was used to search for potential shRNA candidates to knockdown PAH levels in human cells. Potential RNA shRNA sequences were chosen from candidates selected by siRNA or shRNA design programs such as from the GPP Web Portal hosted by the Broad Institute (http://portals.broadinstitute.org/gpp/public/) or the BLOCK-iT RNAi Designer from Thermo Scientific (https://maidesigner.thermofisher.com/maiexpress/). Individual selected shRNA sequences were inserted into a lentiviral vector immediately 3 prime to a RNA polymerase III promoter H1 (SEQ ID NO: 15) to regulate shRNA expression. These lentivirus shRNA constructs were used to transduce cells and measure the change in specific mRNA levels.

Vector Construction: For PAH shRNA, oligonucleotide sequences containing BamHI and EcoRI restriction sites were synthesized by Eurofins MWG Operon. Overlapping sense and antisense oligonucleotide sequences were mixed and annealed during cooling from 70 degrees Celsius to room temperature. The lentiviral vector was digested with the restriction enzymes BamHI and EcoRI for one hour at 37 degrees Celsius. The digested lentiviral vector was purified by agarose gel electrophoresis and extracted from the gel using a DNA gel extraction kit from Thermo Scientific. The DNA concentrations were determined and vector to oligo (3:1 ratio) were mixed, allowed to anneal, and ligated. The ligation reaction was performed with T4 DNA ligase for 30 minutes at room temperature. 2.5 microliters of the ligation mix were added to 25 microliters of STBL3 competent bacterial cells. Transformation was achieved after heat-shock at 42 degrees Celsius. Bacterial cells were spread on agar plates containing ampicillin and drug-resistant colonies (indicating the presence of ampicillin-resistance plasmids) were recovered and expanded in LB broth. To check for insertion of the oligo sequences, plasmid DNA was extracted from harvested bacteria cultures with the Thermo Scientific DNA mini prep kit. Insertion of shRNA sequences in the lentiviral vector was verified by DNA sequencing using a specific primer for the promoter used to regulate shRNA expression. Using the following target sequences, exemplary shRNA sequences were determined to knock-down PAH.

PAH shRNA sequence #1:
(SEQ ID NO: 5)
TCGCATTTCATCAAGATTAATCTCGAGATTAATCTTGATGAAATGCGATT
TTT
PAH shRNA sequence #2:
(SEQ ID NO: 6)
ACTCATAAAGGAGCATATAAGCTCGAGCTTATATGCTCCTTTATGAGTTT
TTT

### Example 3—Phenylalanine Hydroxylase Open Reading Frame Including Complete 5′ and 3′ UTR

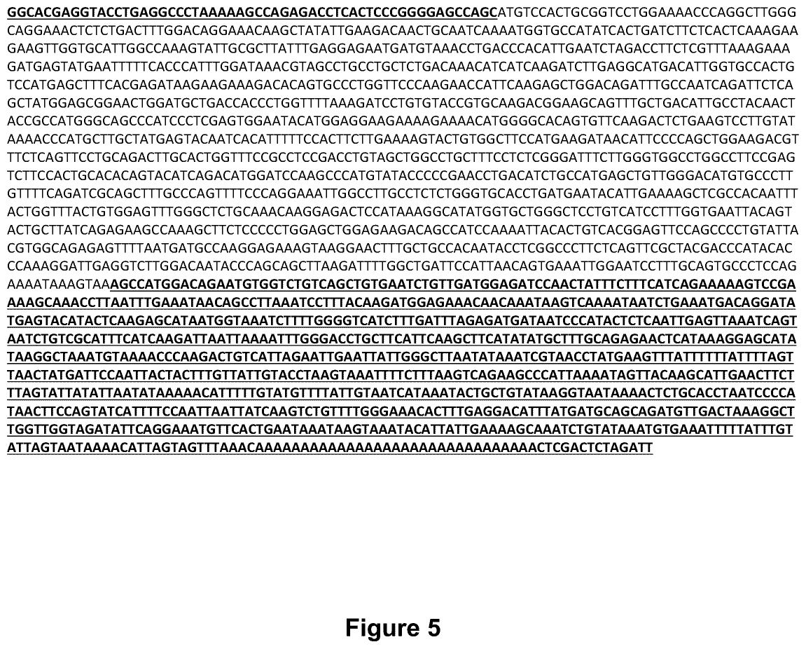

Hepa1-6 mouse hepatoma cells were infected with lentiviral vectors containing the PAH gene (SEQ ID NO: 1), including its full 5 prime untranslated region and its full 3 prime untranslated region (SEQ ID NO: 3) as shown in FIG. 5. FIG. 5 provides the complete DNA sequence of a cDNA expression construct for human PAH (SEQ ID NO: 57). This version includes the intact 5′ UTR region (shown in boldface), the coding region for hPAH, and the complete 3′ UTR (shown in boldface). Results for these infections are detailed in further Examples herein.

### Example 4—Phenylalanine Hydroxylase Open Reading Frame Including Complete 5′ UTR and a Truncated 3′ UTR

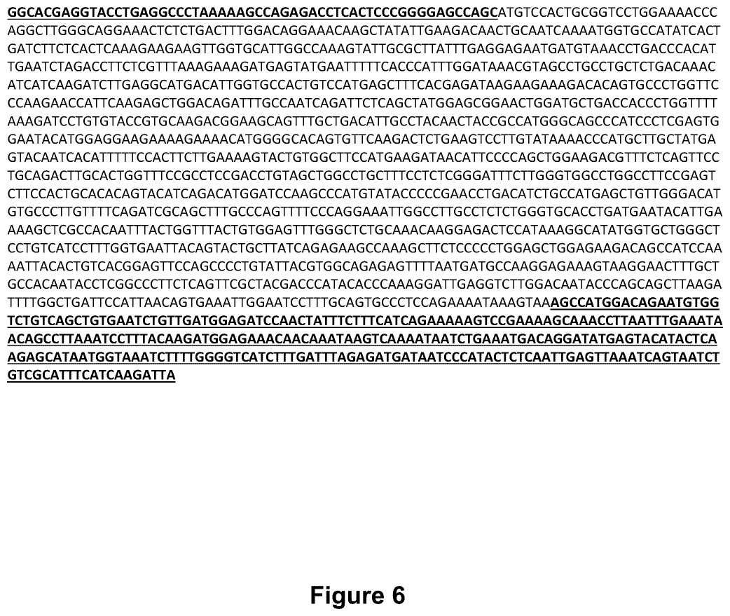

Hepa1-6 mouse hepatoma cells were infected with lentiviral vectors containing the PAH gene (SEQ ID NO: 1), including its full 5 prime untranslated region and a truncated 3 prime untranslated region (SEQ ID NO: 4) as shown in FIG. 6. FIG. 6 provides the cDNA sequence for human PAH that includes the 5′ UTR (897 nucleotides) (shown in boldface), the coding region for hPAH, and the truncated 3′ UTR (289 nucleotides) (shown in boldface) (SEQ ID NO: 58).

### Example 5. Materials and Methods for PAH

The sequence of Homo sapiens phenylalanine hydroxylase (hPAH) mRNA (Gen Bank: NM_000277.1) was chemically synthesized with EcoRI and SalI restriction enzyme sites located at distal and proximal ends of the gene. hPAH treated with EcoRI and SalI restriction enzymes was excised and ligated into pCDH plasmids under control of a hybrid promoter comprising parts of ApoE (NM_000001.11, U35114.1) and hAAT (HG98385.1) locus control regions. Similarly, the mouse PAH gene (mPAH) (NM_008777.3) was synthesized and inserted into pCDH under control of the same hybrid promoter. Additionally, human PAH was synthesized to include the 3′ untranslated region (UTR).

In a further modification, the naturally occurring UTR was truncated to improve expression of the hPAH gene when controlled by liver-specific promoter hAAT. Oligonucleotide sequences containing hPAH, hPAH with full-length UTR, hPAH with truncated UTR or mPAH alone with BamHI and EcoRI restriction sites were synthesized by Eurofins Genomics. Oligonucleotide sequences were annealed by incubation at 70 degrees Celsius and cooling to room temperature. The lentiviral vector was digested with the restriction enzymes BamHI and EcoRI for one hour at 37 degrees Celsius. The digested lentiviral vector was purified by agarose gel electrophoresis and extracted from the gel using a DNA gel extraction kit from Invitrogen. The DNA concentration was determined then mixed with the synthetic oligonucleotides (hPAH or mPAH) using a vector to oligo sequence ratio of 3:1 insert to vector. The mixture was ligated T4 DNA ligase for 30 minutes at room temperature. 2.5 microliters of the ligation mix was added to 25 microliters of STBL3 competent bacterial cells. Transformation was carried out by heat-shock at 42 degrees Celsius. Bacterial cells were streaked onto agar plates containing ampicillin and then colonies were expanded in LB broth. To check for insertion of the oligo sequences, Plasmid DNA was extracted from harvested bacteria cultures with the Invitrogen DNA mini prep kit. Insertion of the shRNA sequence in the lentiviral vector (LV) was verified by DNA sequencing using a primer complementary to the promoter used for shRNA expression. The lentiviral vectors containing a verified hPAH or mPAH sequence were then used to package lentiviral particles to test for their ability to express PAN. Mammalian cells were transduced with lentiviral particles. Cells were collected after 2-4 days and protein was analyzed by western blot for PAH expression.

Modifications of the hPAH Sequence:

Several modifications of the hPAH sequence were incorporated to improve cellular expression levels. First, normal hPAH 3′ untranslated region (UTR) was inserted after the PAH coding region and before the mRNA terminus. This created LV-hAAT-hPAH-UTR. Levels of hPAH expression were increased by adding the 3′ UTR but did not reach the levels of mPAH expressed in a similar vector.

Next, the hPAH UTR region was modified to improve expression levels under the control of a liver-specific promoter. A portion of the untranslated region approximately equal to the distal half of the sequence was removed. This modification increased expression of LV-hAAT-hPAH-UTR up to levels similar to what was achieved for mPAH expression. Surprisingly, truncation of the UTR was only required for high-level expression when using the liver-specific hAAT promoter. Generating hPAH expression constructs under the control of the CMV immediate early promoter gave high-level expression irrespective of the presence or absence of UTR and irrespective of whether or not the UTR was truncated. This important advance in understanding the structure function for the hPAH gene locus allows us to generate constructs for specific expression in liver tissue while still achieving high-level production of hPAH. Restricting transgene expression to liver cells is an important consideration for vector safety and target specificity in a genetic medicine for phenylketonuria.

### Example 6. Immunoblot Analysis Comparing Levels of Expression for Human and Mouse PAH Genes

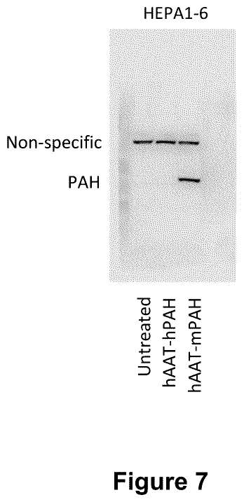

An immunoblot analysis comparing levels of expression for human and mouse PAH genes was conducted as summarized in FIG. 7. This Example illustrates that expression of mouse PAH in Hepa1-6 mouse liver cancer cells (Hepa1-6) is higher compared to the nearly undetectable expression of human PAH in Hepa1-6.

Human and mouse PAH were synthesized and inserted into lentiviral vectors. Insertion of the sequences was then verified by DNA sequencing. The lentiviral vectors containing a correct hPAH or mPAH sequence were then used to transduce Hepa1-6 mouse liver cancer cells (purchased from American Type Culture Collection, Manassas, VA). Cells were collected after 2-4 days and protein was analyzed by western blot for PAH expression. Hepa1-6 cells were infected with lentiviral particles containing green fluorescent protein (GFP) as a marker for transduction efficiency. The relative expression of human or mouse PAH was detected by immunoblot using an anti-PAH antibody (Abcam).

### Example 7. Lentivirus-Delivered Expression of hPAH with or without the 3′ UTR Region in Hepa1-6 Cells

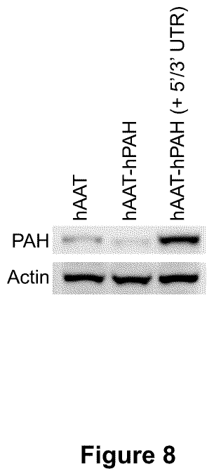

This Example illustrates that expression of PAH is substantially increased in Hepa1-6 carcinoma cells when a lentiviral vector expresses hPAH including both the 5′ UTR and 3′ UTR, as shown in FIG. 8. This Example also illustrates that a lentivirus vector expressing only the coding region for hPAH does not increase the levels of PAH protein in Hepa1-6 cells.

Human PAH was synthesized and inserted into lentiviral vectors. Insertion of the sequences was verified by DNA sequencing. The lentiviral vectors containing a verified hPAH sequence were then used to transduce Hepa1-6 mouse liver cancer cells (purchased from American Type Culture Collection, Manassas, VA). The lentiviral vectors incorporated a human PAH gene with or without its 3′ UTR. In addition, hPAH expression in these constructs was driven by the hAAT promoter. Cells were transduced with lentiviral particles and after 2-4 days protein was analyzed by western blot for PAH expression. Hepa1-6 cells were infected with lentiviral particles containing green fluorescent protein (GFP) as a marker for transduction efficiency. The relative expression of human PAH was detected by immunoblot using an anti-PAH antibody (Abcam).

As shown in FIG. 8, three groups are compared: a control comprising Hepa1-6 cells alone (lane 1), a group expressing a lentivirus vector expressing only the coding region for hPAH (lane 2), and a lentivirus vector expressing hPAH and including both the 5′ and 3′ UTR regions (lane 3). Notably, Hepa1-6 carcinoma cells are derived from mouse liver tissue and thus there is a natural background expression of PAH observed in Lane 1 (labeled hAAT). FIG. 8 demonstrates that expression of PAH is substantially increased in Hepa1-6 carcinoma cells when a lentivirus (LV) expressing PAH shRNA includes both the 5′ UTR and 3′ UTR.

### Example 8. Lentiviral Vector Expressing hPAH with a Truncated 3′ UTR in Hepa1-6 Cells

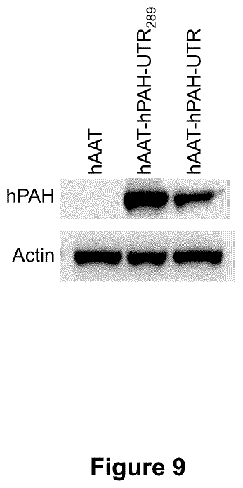

This Example illustrates that a lentiviral vector expressing hPAH with a truncated 3′ UTR (hPAH-3′UTR) demonstrates substantially increased expression of hPAH compared to constructs containing a full-length 3′UTR sequence, as shown in FIG. 9.

Human PAH was synthesized and inserted into lentiviral vectors. Insertion of the sequences was verified by DNA sequencing. The lentiviral vectors containing a verified hPAH sequence were then used to transduce Hepa1-6 liver cancer cells (purchased from American Type Culture Collection, Manassas, VA). The lentiviral vectors incorporated a human PAH gene with or without its 3′ UTR. In addition, hPAH expression in these constructs was driven by the hAAT promoter. Cells were transduced with lentiviral particles and after 2-4 days protein was analyzed by western blot for PAH expression. Hepa1-6 cells were infected with lentiviral particles containing green fluorescent protein (GFP) as a marker for transduction efficiency. The relative expression of human PAH was detected by immunoblot using an anti-PAH antibody (Abcam) and the loading control Beta-actin.

FIG. 9 shows expression of hPAH constructs in Hepa1-6 carcinoma cells. As shown in FIG. 9, three groups are compared: a control lentiviral vector expressing only the coding region for hPAH (lane 1), a construct containing hPAH-3′UTR (lane 2), and a full-length hPAH 3′UTR sequence (lane 3). Notably, Hepa1-6 carcinoma cells are derived from human liver tissue and thus there is a natural background expression of PAH observed in Lane 1 (labeled hAAT). This Example illustrates that hPAH-3′UTR increases hPAH expression relative to the wild type 3′UTR sequence in Hepa1-6 cells.

### Example 9. Expression of Codon-Optimized hPAH with or without WPRE in Mouse Hepa1-6

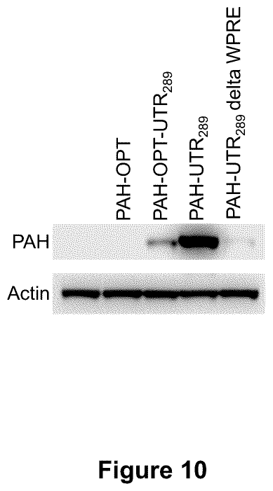

This Example illustrates that removing the WPRE element from a lentiviral vector containing the hAAT-hPAH-3′UTR₂₈₉ reduced hPAH expression significantly, indicating that WPRE is required for optimal protein expression, as shown in FIG. 10. This Example also illustrates that optimizing codon choice based on preferred human codon bias (PAH-OPT) failed to increase hPAH expression levels.

Human PAH was synthesized and inserted into lentiviral vectors. Insertion of the sequences was verified by DNA sequencing. Lentiviral vectors containing a verified hPAH sequence were then used to transduce mouse Hepa1-6 cells (purchased from American Type Culture Collection, Manassas, VA). In addition, hPAH expression in these constructs was driven by the hAAT promoter. Cells were transduced with lentiviral particles and after 2-4 days protein was analyzed by western blot for PAH expression. The relative expression of human PAH was detected by immunoblot using an anti-PAH antibody (Abcam) and the loading control Beta-actin.

This Example shows the effect on hPAH expression in Hepa1-6 cells of: 1) codon optimization of the hPAH coding region and, 2) deletion of the WPRE gene component. Expression of various hPAH constructs in mouse Hepa1-6 cells was compared to address this question. As shown in FIG. 10, five groups are compared: a Beta-actin loading control (lane 1), an optimized codon control construct (lane 2), an optimized codon construct containing a truncated hPAH 3′UTR sequence (lane 3), a control construct containing a truncated hPAH 3′UTR sequence (lane 4), and a construct with a deleted WPRE sequence and containing a truncated hPAH 3′UTR (lane 5). Hepa1-6 carcinoma cells are derived from mouse liver tissue and thus there is a natural background expression of PAH observed in Lane 1 (labeled hAAT). This Example illustrates that optimizing codon choice based on preferred human codon bias (PAH-OPT) failed to increase hPAH expression levels. As observed with the wild type hPAH gene, including a truncated 3′UTR (UTR₂₈₉) increases hPAH expression but only to levels substantially below what is observed with the wild type (non-optimized) sequence linked to UTR₂₈₉. Removing the WPRE element from a lentiviral vector containing the hAAT-hPAH-3′UTR₂₈₉ also reduces hPAH expression indicating that WPRE is required for optimal protein expression.

### Example 10. shPAH-1 and shPAH-2 Reduces hPAH Expression in Human Hep3B Cells

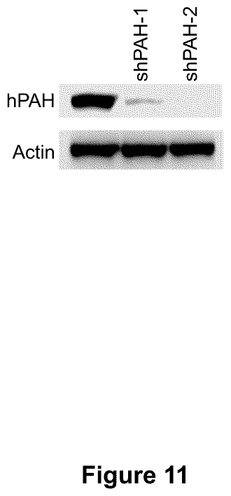

This Example demonstrates that lentivirus-delivered PAH shRNA reduces hPAH expression in human Hep3B cells, as shown in FIG. 11.

Human PAH was synthesized and inserted into lentiviral vectors. Insertion of the sequences was verified by DNA sequencing. The lentiviral vectors containing hPAH sequence was then used to transduce human Hep3B cells (purchased from American Type Culture Collection, Manassas, VA). In addition, hPAH expression in these constructs was driven by the hAAT promoter. Cells were transduced with lentiviral particles and after 2-4 days protein was analyzed by western blot for PAH expression. Insertion of the shRNA sequence in the lentiviral vector (LV) was verified by DNA sequencing using a primer complementary to the promoter used to regulate shRNA expression. The relative expression of human PAH was detected by immunoblot using an anti-PAH antibody (Abcam) and the loading control Beta-actin.

FIG. 11 compares the ability of 2 different shRNA constructs to reduce hPAH expression in Hep3B cells, namely PAH shRNA sequence #1 (shPAH-1) and PAH shRNA sequence #2 (shPAH-2). As shown in FIG. 11, three constructs are compared: a control with Hep3B cells alone (lane 1), a construct containing Hep3B cells plus shPAH-1 (lane 2), and a construct containing Hep3B cells plus shPAH-2 (lane 3). Notably, Hep3B cells express endogenous PAH at significant levels. This Example illustrates that both shPAH-1 and shPAH-2 were effective in reducing endogenous hPAH expression levels.

### Example 11. shPAH-1 Suppression of Endogenous hPAH and hAAT-hPAH-3′UTR₂₈₉ in Hep3B Cells

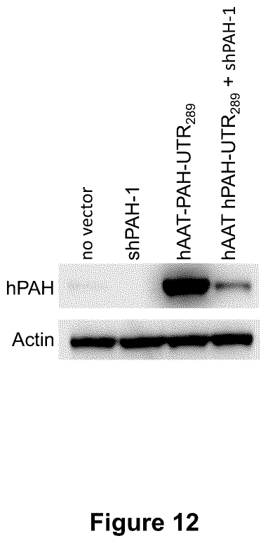

This Example demonstrates that shPAH-1 suppresses expression of endogenous PAH and truncated hPAH 3′UTR (hAAT-hPAH-3′UTR₂₈₉) in Hep3B cells, as shown in FIG. 12.

Human PAH was synthesized and inserted into lentiviral vectors. Insertion of the sequences was verified by DNA sequencing. Lentiviral vectors containing hPAH sequence was then used to transduce human Hep3B cells (purchased from American Type Culture Collection, Manassas, VA). Cells were transduced with lentiviral particles and after 2-4 days protein was analyzed by western blot for PAH expression. The relative expression of human PAH was detected by immunoblot with an anti-PAH antibody (Abcam) and the loading control Beta-actin. hPAH expression in both full-length and 3′UTR-truncated constructs were driven by hAAT promoter. The lentiviral vectors incorporated, in various instances, a human PAH gene with its 3′UTR, a human PAH gene with a truncated 3′UTR, and/or shPAH-1. Insertion of the shRNA sequence in the lentiviral vector (LV) was verified by DNA sequencing using a primer complementary to the promoter used to regulate shRNA expression. The target sequence for shPAH-1 is in the portion of 3′UTR that is preserved in both full-length and shortened versions.

FIG. 12 shows expression of hPAH and PAH shRNA in human Hep3B cells. As shown in FIG. 12, four groups are compared: a control comprising Hep3B cells alone (lane 1), a group comprising Hep3B cells plus a lentiviral vector expressing shPAH-1 (lane 2), a control comprising hAAT-hPAH-3′UTR₂₈₉ alone (lane 3), and a group containing both hAAT-hPAH-3′UTR₂₈₉ and a lentiviral vector expressing shPAH-1. Notably, Hep3B cells alone express endogenous PAH at significant levels. This Example illustrates that shPAH-1 suppresses expression of both endogenous PAH and expression of hAAT-hPAH-3′UTR₂₈₉. Further, this confirms the significant potency of shPAH-1 against endogenous and hAAT-hPAH-3′UTR₂₈₉ in Hep3B cells.

### Example 12. shPAH-2 Suppression of Endogenous hPAH but not hAAT-hPAH-3′UTR₂₈₉ in HepG2 Cells

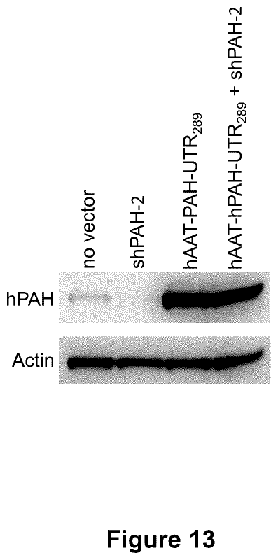

This Example illustrates that shPAH-2 suppresses expression of endogenous PAH but does not suppress expression of hAAT-hPAH-3′UTR₂₈₉ in HepG2 cells, as shown in FIG. 13.

Human PAH was synthesized and inserted into lentiviral vectors. Insertion of the sequences was verified by DNA sequencing. Lentiviral vectors containing hPAH sequence was then used to transduce human Hep3B cells (purchased from American Type Culture Collection, Manassas, VA). Cells were transduced with lentiviral particles and after 2-4 days protein was analyzed by western blot for PAH expression. The relative expression of human PAH was detected by immunoblot using an anti-PAH antibody (Abcam) and the loading control Beta-actin. hPAH expression in both full-length and 3′UTR-truncated constructs were driven by hAAT promoter. The lentiviral vectors incorporated, in various instances, a human PAH gene with its 3′UTR, a human PAH gene with a truncated 3′UTR, and/or shPAH-2. Insertion of the shRNA sequence in the lentiviral vector (LV) was verified by DNA sequencing using a primer complementary to the promoter used to regulate shPAH-2 expression. The target sequence for shPAH-2 is in the distal portion of the hPAH 3′UTR that is present in full-length hPAH construct but absent in the truncated hPAH construct (hAAT-hPAH-3′UTR₂₈₉).

FIG. 13 shows expression of hPAH and PAH shRNA in human Hep3B cells. As shown in FIG. 13, four groups are compared: a control with HepG2 cells alone (lane 1), HepG2 cells plus a lentiviral vector expressing shPAH-2 (lane 2), a control with lentiviral vector expressing truncated hPAH 3′UTR (lane 3), and a lane containing both a lentiviral vector expressing truncated hPAH 3′UTR (hPAH-3′UTR) and a lentiviral vector expressing shPAH-2. This Example illustrates that the shPAH-2 sequence suppresses expression of endogenous PAH but has no discernible effect on expression of hAAT-hPAH-3′UTR₂₈₉.

### Example 13. Preliminary Test of hAAT-PAH-UTR in the Pah(enu2) Mouse

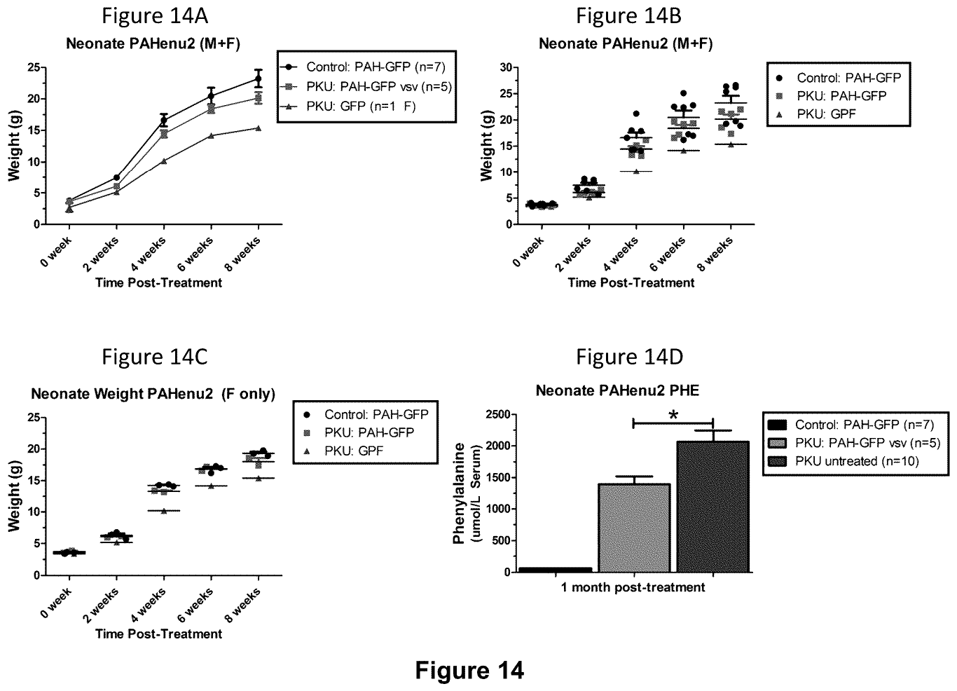

FIG. 14 summarizes results from a preliminary test of hAAT-PAH-UTR in the Pah(enu2) mouse that is a standard model for experimental studies on PKU (Shedlovsky, McDonald et al. 1993, Fang, Eisensmith et al. 1994, Mochizuki, Mizukami et al. 2004, Oh, Park et al. 2004). Panel A shows that lentivirus vector hAAT-PAH injected directly into the liver of neonatal Pah(enu2) mice substantially corrects a growth defect seen in mice that received only a control lentivirus vector that does not express PAH. Panel B provides a cluster plot representation of data in Panel A showing a clear overlap between weight gain curves for normal mice and Pah(enu2) treated with LV-hAAT-PAH. Panel C shows that LV-hAAT-PAH was effective in female mice. This is important because the PAH defect in females is more difficult to correct compared to male mice. Panel D plots the plasma phenylalanine levels for control (normal) mice, Pah(enu2) mice treated with LV-hAAT-PAH and Pah(enu2) mice treated with a control lentivirus vector that does not express PAH.

### Experimental Methodology:

Neonatal mice aged 1 to 2 days were divided into three groups of four neonatal mice each. The first group of neonatal mice comprise a control group with normal PAH expression activity. The second and third group of neonatal mice contain the mutation PAH(enu2), which is a chemically induced mutation in the PAH gene that inhibits enzymatic activity of PAH.

The first group of neonatal mice were injected with lentiviral vectors comprising the hAAT promoter, human PAH, an elongation factor (EF1), and green fluorescent protein (GFP). The second group of neonatal mice were injected with lentiviral vectors lacking human PAH but comprising the hAAT promoter, an elongation factor (EF1), and green fluorescent protein (GFP). The third group of neonatal mice were injected with lentiviral vectors comprising the hAAT promoter, human PAH, an elongation factor (EF1), and green fluorescent protein (GFP).

Neonatal mice were injected with 10 μL of a lentivirus particle suspension containing between 1×10⁶ to 1×10¹⁰ transducing units per mL of normal saline or blood plasma substitute directly into the liver. Prior to injection, neonatal mice were treated with clodronate liposomes to deplete liver Kupffer cells.

Neonatal mice were monitored for phenotypic changes associated with reduced phenylalanine levels in the blood, including coat color changes, PAH and phenylalanine levels, and behavior. At 0, 4, and 8 weeks post-injection, blood phenylalanine levels were measured. At 0, 2, 4, and 8 weeks post-injection, neonatal mice weight were measured. If the growth of neonatal mice in group three improved over growth of neonatal mice in group two, behavioral tests will be performed, including the T-maze Spontaneous Alternation Test and the Win-Stay Eight-arm Radial Maze Task. At 8 weeks post-injection, two mice from each group will be sacrificed and human PAH expression in the liver will be measured. Methylome assessment and long bone and spinal bone assessments will be performed on sacrificed mice. The remaining mice were maintained and blood phenylalanine will was measured at 6 months post-injection.

### Example 14. Lentiviral-Delivered Expression of the Human PAH Gene Using hAAT and CMV Promoters in Hepa1-6 Mouse Hepatoma Cells

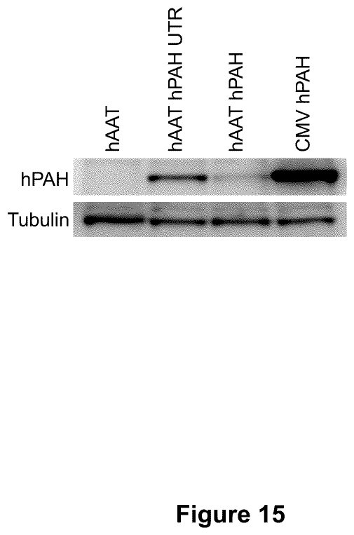

This Example illustrates that utilization of hPAH expression constructs under control of the CMV immediate early promoter provides high-level expression irrespective of the presence or absence of 3′UTR and irrespective of whether or not the 3′UTR is truncated, as shown in FIG. 15.

Human PAH was synthesized and inserted into lentiviral vectors. Insertion of the sequences was verified by DNA sequencing. Lentiviral vectors containing hPAH sequence was then used to transduce human Hep3B cells (purchased from American Type Culture Collection, Manassas, VA). Cells were transduced with lentiviral particles and after 2-4 days protein was analyzed by western blot for PAH expression. The relative expression of human PAH was detected by immunoblot using an anti-PAH antibody (Abcam) or an anti-tubulin antibody (Sigma) as the loading control. hPAH expression in both full-length and 3′UTR-truncated constructs were driven by hAAT promoter or CMV promoter, respectively. The lentiviral vectors incorporated, in various instances, a human PAH gene with its 3′UTR, a human PAH gene with a truncated 3′UTR, in the absence or presence of hAAT promoter or CMV promoter.

As shown in FIG. 15, four groups are compared: a control with Hepa1-6 cells and hAAT promoter alone (lane 1), a lentiviral vector expressing fill-length 3′UTR hPAH under control of the hAAT promoter (lane 2), a lentiviral vector expressing truncated 3′UTR hPAH under control of the hAAT promoter (lane 3), and a group with a lentiviral vector expressing truncated 3′UTR hPAH under control of the CMV promoter. This Example illustrates that hPAH expression under control of the CMV immediate early promoter gives rise to high-level expression irrespective of the presence or absence of UTR and irrespective of whether or not the UTR is truncated. This permits the generation of constructs for specific expression in liver tissue while still achieving high-level production of hPAH. Notably, restricting transgene expression to liver cells is an important consideration for vector safety and target specificity in a genetic medicine for phenylketonuria.

### Example 15. Lentivirus-Delivered Expression of hPAH Using Expression Constructs with the hAAT Promoter and Liver-Specific Enhancer Element ApoE (1), ApoE (2), or Prothrombin in Mouse Hepa1-6 Cells

This Example illustrates that ApoE (1), ApoE (2), and prothrombin enhancers may be utilized to increase expression of PAH in mouse Hepa1-6 cells.

Human PAH was synthesized and inserted into lentiviral vectors. Insertion of the sequences was verified by DNA sequencing. Lentiviral vectors containing hPAH sequence was then used to transduce human Hep3B cells (purchased from American Type Culture Collection, Manassas, VA). Cells were transduced with lentiviral particles and after 2-4 days protein was analyzed by western blot for PAH expression. PAH was detected by immunoblot using an anti-PAH antibody and an anti-Beta actin antibody for the loading control.

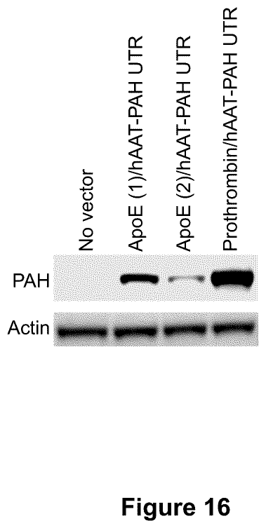

As shown in FIG. 16, four groups are compared: a control with Hepa1-6 cells alone (lane 1), a lentiviral vector expressing ApoE(1) enhancer with full-length 3′UTR hPAH under control of the hAAT promoter (lane 2), a lentiviral vector expressing ApoE(2) enhancer with full-length 3′UTR hPAH under control of the hAAT promoter (lane 3), and a lentiviral vector expressing the prothrombin enhancer with full-length 3′UTR hPAH under control of the hAAT promoter (lane 3). This Example illustrates that ApoE (1), ApoE (2), and prothrombin enhancers may each be utilized to increase expression of PAH in mouse Hepa1-6 cells under control of the hAAT promoter.

The disclosure of the above example embodiments is intended to be illustrative, but not limiting, of the scope of the inventions, which are set forth in the following claims and their equivalents. Although example embodiments of the inventions have been described in some detail for purposes of clarity of understanding, it will be apparent that certain changes and modifications can be practiced within the scope of the following claims. In the following claims, elements and/or steps do not imply any particular order of operation, unless explicitly stated in the claims or implicitly required by the disclosure.

Sequence Listings

| SEQ ID NO: | Description | Sequence |
|---|---|---|
| 1 | PAH | ATGTCCACTGCGGTCCTGGAAAACCCAGGCTTGGGCAGGAAACTCT |
| | | CTGACTTTGGACAGGAAACAAGCTATATTGAAGACAACTGCAATCA |
| | | AAATGGTGCCATATCACTGATCTTCTCACTCAAAGAAGAAGTTGGT |
| | | GCATTGGCCAAAGTATTGCGCTTATTTGAGGAGAATGATGTAAACC |
| | | TGACCCACATTGAATCTAGACCTTCTCGTTTAAAGAAAGATGAGTA |
| | | TGAATTTTTCACCCATTTGGATAAACGTAGCCTGCCTGCTCTGACA |
| | | AACATCATCAAGATCTTGAGGCATGACATTGGTGCCACTGTCCATG |
| | | AGCTTTCACGAGATAAGAAGAAAGACACAGTGCCCTGGTTCCCAAG |
| | | AACCATTCAAGAGCTGGACAGATTTGCCAATCAGATTCTCAGCTAT |
| | | GGAGCGGAACTGGATGCTGACCACCCTGGTTTTAAAGATCCTGTGT |
| | | ACCGTGCAAGACGGAAGCAGTTTGCTGACATTGCCTACAACTACCG |
| | | CCATGGGCAGCCCATCCCTCGAGTGGAATACATGGAGGAAGAAAAG |
| | | AAAACATGGGGCACAGTGTTCAAGACTCTGAAGTCCTTGTATAAAA |
| | | CCCATGCTTGCTATGAGTACAATCACATTTTTCCACTTCTTGAAAA |
| | | GTACTGTGGCTTCCATGAAGATAACATTCCCCAGCTGGAAGACGTT |
| | | TCTCAATTCCTGCAGACTTGCACTGGTTTCCGCCTCCGACCTGTGG |
| | | CTGGCCTGCTTTCCTCTCGGGATTTCTTGGGTGGCCTGGCCTTCCG |
| | | AGTCTTCCACTGCACACAGTACATCAGACATGGATCCAAGCCCATG |
| | | TATACCCCCGAACCTGACATCTGCCATGAGCTGTTGGGACATGTGC |
| | | CCTTGTTTTCAGATCGCAGCTTTGCCCAGTTTTCCCAGGAAATTGG |
| | | CCTTGCCTCTCTGGGTGCACCTGATGAATACATTGAAAAGCTCGCC |
| | | ACAATTTACTGGTTTACTGTGGAGTTTGGGCTCTGCAAACAAGGAG |
| | | ACTCCATAAAGGCATATGGTGCTGGGCTCCTGTCATCCTTTGGTGA |
| | | ATTACAGTACTGCTTATCAGAGAAGCCAAAGCTTCTCCCCCTGGAG |
| | | CTGGAGAAGACAGCCATCCAAAATTACACTGTCACGGAGTTCCAGC |
| | | CCCTGTATTACGTGGCAGAGAGTTTTAATGATGCCAAGGAGAAAGT |
| | | AAGGAACTTTGCTGCCACAATACCTCGGCCCTTCTCAGTTCGCTAC |
| | | GACCCATACACCCAAAGGATTGAGGTCTTGGACAATACCCAGCAGC |
| | | TTAAGATTTTGGCTGATTCCATTAACAGTGAAATTGGAATCCTTTG |
| | | CAGTGCCCTCCAGAAAATAAAGTAA |
| 2 | Codon | ATGAGCACAGCTGTGTTGGAAAATCCTGGGCTGGGCCGTAAGCTTT |
| | optimized | CCGATTTCGGCCAGGAGACTTCATACATTGAGGACAACTGCAACCA |
| | PAH | GAATGGGGCCATTTCTTTGATCTTCAGTCTCAAAGAAGAGGTAGGC |
| | | GCTCTGGCTAAGGTCCTGAGGCTGTTTGAGGAAAATGACGTGAATC |
| | | TGACACACATTGAGTCTAGGCCTTCCCGACTTAAGAAGGATGAGTA |
| | | TGAGTTCTTCACACACCTGGACAAACGATCTCTCCCAGCACTGACC |
| | | AATATCATCAAGATTCTCAGGCATGATATCGGTGCCACGGTCCACG |
| | | AACTTTCACGCGATAAGAAGAAAGACACAGTTCCCTGGTTCCCGAG |
| | | AACCATTCAGGAACTGGATAGGTTTGCCAATCAGATTCTGAGCTAT |
| | | GGGGCAGAGTTGGATGCCGACCATCCAGGCTTCAAAGACCCCGTAT |
| | | ATCGGGCTCGGAGAAAGCAGTTTGCAGACATCGCTTACAATTACAG |
| | | GCATGGACAGCCCATCCCTAGAGTGGAGTACATGGAAGAAGGCAAG |
| | | AAAACCTGGGGAACGGTGTTTAAGACCCTCAAAAGCCTGTATAAGA |
| | | CCCACGCGTGTTATGAGTACAACCACATTTTCCCATTGCTGGAGAA |
| | | GTACTGTGGCTTTCACGAGGACAACATCCCTCAACTGGAGGATGTT |
| | | TCACAGTTCCTTCAGACTTGCACTGGTTTCCGCCTTCGACCTGTGG |
| | | CTGGGCTGCTTAGCTCACGGGACTTCCTGGGAGGCCTGGCCTTCAG |
| | | AGTCTTTCACTGCACTCAGTACATTCGGCATGGCTCTAAGCCAATG |
| | | TACACCCCTGAACCGGATATATGCCACGAGCTGTTGGGACATGTGC |
| | | CCCTGTTTTCTGATCGCAGCTTTGCCCAGTTTTCCCAGGAGATTGG |
| | | CCTGGCAAGTCTTGGTGCGCCTGATGAGTACATCGAGAAGCTCGCG |
| | | ACAATCTACTGGTTCACCGTGGAATTTGGACTCTGCAAACAAGGGG |
| | | ACTCTATCAAAGCCTACGGAGCAGGACTCCTCTCCAGCTTCGGTGA |
| | | ACTGCAGTATTGTCTGTCCGAGAAACCCAAACTCTTGCCCCTGGAA |
| | | CTGGAAAAGACTGCCATCCAAAACTATACTGTCACGGAATTTCAGC |
| | | CACTGTATTATGTGGCTGAATCCTTTAACGATGCCAAGGAGAAGGT |
| | | CCGTAATTTTGCTGCCACAATACCACGCCCCTTCAGCGTGAGATAC |
| | | GACCCGTATACACAACGGATAGAGGTTCTGGACAACACCCAGCAAC |
| | | TGAAAATTCTGGCAGACAGTATAAACAGCGAAATAGGGATCCTCTG |
| | | TAGTGCCCTGCAGAAAATCAAATGA |
| 3 | PAH 3′UTR | AGCCATGGACAGAATGTGGTCTGTCAGCTGTGAATCTGTTGATGGA |
| | sequence (897 | GATCCAACTATTTCTTTCATCAGAAAAAGTCCGAAAAGCAAACCTT |
| | nucleotides) | AATTTGAAATAACAGCCTTAAATCCTTTACAAGATGGAGAAACAAC |
| | | AAATAAGTCAAAATAATCTGAAATGACAGGATATGAGTACATACTC |
| | | AAGAGCATAATGGTAAATCTTTTGGGGTCATCTTTGATTTAGAGAT |
| | | GATAATCCCATACTCTCAATTGAGTTAAATCAGTAATCTGTCGCAT |
| | | TTCATCAAGATTAATTAAAATTTGGGACCTGCTTCATTCAAGCTTC |
| | | ATATATGCTTTGCAGAGAACTCATAAAGGAGCATATAAGGCTAAAT |
| | | GTAAAACCCAAGACTGTCATTAGAATTGAATTATTGGGCTTAATAT |
| | | AAATCGTAACCTATGAAGTTTATTTTTTATTTTAGTTAACTATGAT |
| | | TCCAATTACTACTTTGTTATTGTACCTAAGTAAATTTTCTTTAAGT |
| | | CAGAAGCCCATTAAAATAGTTACAAGCATTGAACTTCTTTAGTATT |
| | | ATATTAATATAAAAACATTTTTGTATGTTTTATTGTAATCATAAAT |
| | | ACTGCTGTATAAGGTAATAAAACTCTGCACCTAATCCCCATAACTT |
| | | CCAGTATCATTTTCCAATTAATTATCAAGTCTGTTTTGGGAAACAC |
| | | TTTGAGGACATTTATGATGCAGCAGATGTTGACTAAAGGCTTGGTT |
| | | GGTAGATATTCAGGAAATGTTCACTGAATAAATAAGTAAATACATT |
| | | ATTGAAAAGCAAATCTGTATAAATGTGAAATTTTTATTTGTATTAG |
| | | TAATAAAACATTAGTAGTTTAAACAAAAAAAAAAAAAAAAAAAAAA |
| | | AAAAAAAAACTCGACTCTAGATT |
| 4 | PAH 3′UTR | AGCCATGGACAGAATGTGGTCTGTCAGCTGTGAATCTGTTGATGGA |
| | sequence (289 | GATCCAACTATTTCTTTCATCAGAAAAAGTCCGAAAAGCAAACCTT |
| | nucleotides) | AATTTGAAATAACAGCCTTAAATCCTTTACAAGATGGAGAAACAAC |
| | | AAATAAGTCAAAATAATCTGAAATGACAGGATATGAGTACATACTC |
| | | AAGAGCATAATGGTAAATCTTTTGGGGTCATCTTTGATTTAGAGAT |
| | | GATAATCCCATACTCTCAATTGAGTTAAATCAGTAATCTGTCGCAT |
| | | TTCATCAAGATTA |
| 5 | PAH shRNA | TCGCATTTCATCAAGATTAATCTCGAGATTAATCTTGATGAAATGC |
| | sequence #1 | GATTTTT |
| 6 | PAH shRNA | ACTCATAAAGGAGCATATAAGCTCGAGCTTATATGCTCCTTTATGA |
| | sequence #2 | GTTTTTT |
| 7 | Rous Sarcoma | GTAGTCTTATGCAATACTCTTGTAGTCTTGCAACATGGTAACGATG |
| | virus (RSV) | AGTTAGCAACATGCCTTACAAGGAGAGAAAAAGCACCGTGCATGCC |
| | promoter | GATTGGTGGAAGTAAGGTGGTACGATCGTGCCTTATTAGGAAGGCA |
| | | ACAGACGGGTCTGACATGGATTGGACGAACCACTGAATTGCCGCAT |
| | | TGCAGAGATATTGTATTTAAGTGCCTAGCTCGATACAATAAACG |
| 8 | 5′ Long | GGTCTCTCTGGTTAGACCAGATCTGAGCCTGGGAGCTCTCTGGCTA |
| | terminal | ACTAGGGAACCCACTGCTTAAGCCTCAATAAAGCTTGCCTTGAGTG |
| | repeat (LTR) | CTTCAAGTAGTGTGTGCCCGTCTGTTGTGTGACTCTGGTAACTAGA |
| | | GATCCCTCAGACCCTTTTAGTCAGTGTGGAAAATCTCTAGCA |
| 9 | Psi Packaging | TACGCCAAAAATTTTGACTAGCGGAGGCTAGAAGGAGAGAG |
| | signal | |
| 10 | Rev response | AGGAGCTTTGTTCCTTGGGTTCTTGGGAGCAGCAGGAAGCACTATG |
| | element (RRE) | GGCGCAGCCTCAATGACGCTGACGGTACAGGCCAGACAATTATTGT |
| | | CTGGTATAGTGCAGCAGCAGAACAATTTGCTGAGGGCTATTGAGGC |
| | | GCAACAGCATCTGTTGCAACTCACAGTCTGGGGCATCAAGCAGCTC |
| | | CAGGCAAGAATCCTGGCTGTGGAAAGATACCTAAAGGATCAACAGC |
| | | TCC |
| 11 | Central | TTTTAAAAGAAAAGGGGGGATTGGGGGGTACAGTGCAGGGGAAAGA |
| | polypurine | ATAGTAGACATAATAGCAACAGACATACAAACTAAAGAATTACAAA |
| | tract (cPPT) | AACAAATTACAAAATTCAAAATTTTA |
| 12 | Human alpha-1 | GATCTTGCTACCAGTGGAACAGCCACTAAGGATTCTGCAGTGAGAG |
| | antitrypsin | CAGAGGGCCAGCTAAGTGGTACTCTCCCAGAGACTGTCTGACTCAC |
| | promoter | GCCACCCCCTCCACCTTGGACACAGGACGCTGTGGTTTCTGAGCCA |
| | (hAAT) | GGTACAATGACTCCTTTCGGTAAGTGCAGTGGAAGCTGTACACTGC |
| | | CCAGGCAAAGCGTCCGGGCAGCGTAGGCGGGCGACTCAGATCCCAG |
| | | CCAGTGGACTTAGCCCCTGTTTGCTCCTCCGATAACTGGGGTGACC |
| | | TTGGTTAATATTCACCAGCAGCCTCCCCCGTTGCCCCTCTGGATCC |
| | | ACTGCTTAAATACGGACGAGGACAGGGCCCTGTCTCCTCAGCTTCA |
| | | GGCACCACCACTGACCTGGGACAGTGAAT |
| 13 | Long WPRE | AATCAACCTCTGATTACAAAATTTGTGAAAGATTGACTGGTATTCT |
| | sequence | TAACTATGTTGCTCCTTTTACGCTATGTGGATACGCTGCTTTAATG |
| | | CCTTTGTATCATGCTATTGCTTCCCGTATGGCTTTCATTTTCTCCT |
| | | CCTTGTATAAATCCTGGTTGCTGTCTCTTTATGAGGAGTTGTGGCC |
| | | CGTTGTCAGGCAACGTGGCGTGGTGTGCACTGTGTTTGCTGACGCA |
| | | ACCCCCACTGGTTGGGGCATTGCCACCACCTGTCAGCTCCTTTCCG |
| | | GGACTTTCGCTTTCCCCCTCCCTATTGCCACGGCGGAACTCATCGC |
| | | CGCCTGCCTTGCCCGCTGCTGGACAGGGGCTCGGCTGTTGGGCACT |
| | | GACAATTCCGTGGTGTTGTCGGGGAAATCATCGTCCTTTCCTTGGC |
| | | TGCTCGCCTGTGTTGCCACCTGGATTCTGCGCGGGACGTCCTTCTG |
| | | CTACGTCCCTTCGGCCCTCAATCCAGCGGACCTTCCTTCCCGCGGC |
| | | CTGCTGCCGGCTCTGCGGCCTCTTCCGCGTCTTCGCCTTCGCCCTC |
| | | AGACGAGTCGGATCTCCCTTTGGGCCGCCTCCCCGCCT |
| 14 | 3′delta LTR | TGGAAGGGCTAATTCACTCCCAACGAAGATAAGATCTGCTTTTTGC |
| | | TTGTACTGGGTCTCTCTGGTTAGACCAGATCTGAGCCTGGGAGCTC |
| | | TCTGGCTAACTAGGGAACCCACTGCTTAAGCCTCAATAAAGCTTGC |
| | | CTTGAGTGCTTCAAGTAGTGTGTGCCCGTCTGTTGTGTGACTCTGG |
| | | TAACTAGAGATCCCTCAGACCCTTTTAGTCAGTGTGGAAAATCTCT |
| | | AGCAGTAGTAGTTCATGTCA |
| 15 | H1 Promoter | GAACGCTGACGTCATCAACCCGCTCCAAGGAATCGCGGGCCCAGTG |
| | | TCACTAGGCGGGAACACCCAGCGCGCGTGCGCCCTGGCAGGAAGAT |
| | | GGCTGTGAGGGACAGGGGAGTGGCGCCCTGCAATATTTGCATGTCG |
| | | CTATGTGTTCTGGGAAATCACCATAAACGTGAAATGTCTTTGGATT |
| | | TGGGAATCTTATAAGTTCTGTATGAGACCACTT |
| 16 | CAG | TAGTTATTAATAGTAATCAATTACGGGGTCATTAGTTCATAGCCCA |
| | promoter | TATATGGAGTTCCGCGTTACATAACTTACGGTAAATGGCCCGCCTG |
| | | GCTGACCGCCCAACGACCCCCGCCCATTGACGTCAATAATGACGTA |
| | | TGTTCCCATAGTAACGCCAATAGGGACTTTCCATTGACGTCAATGG |
| | | GTGGACTATTTACGGTAAACTGCCCACTTGGCAGTACATCAAGTGT |
| | | ATCATATGCCAAGTACGCCCCCTATTGACGTCAATGACGGTAAATG |
| | | GCCCGCCTGGCATTATGCCCAGTACATGACCTTATGGGACTTTCCT |
| | | ACTTGGCAGTACATCTACGTATTAGTCATCGCTATTACCATGGGTC |
| | | GAGGTGAGCCCCACGTTCTGCTTCACTCTCCCCATCTCCCCCCCCT |
| | | CCCCACCCCCAATTTTGTATTTATTTATTTTTTAATTATTTTGTGC |
| | | AGCGATGGGGGCGGGGGGGGGGGGGGCGCGCGCCAGGCGGGGCGGG |
| | | GCGGGGCGAGGGGCGGGGCGGGGCGAGGCGGAGAGGTGCGGCGGCA |
| | | GCCAATCAGAGCGGCGCGCTCCGAAAGTTTCCTTTTATGGCGAGGC |
| | | GGCGGCGGCGGCGGCCCTATAAAAAGCGAAGCGCGCGGCGGGCG |
| 17 | HIV Gag | ATGGGTGCGAGAGCGTCAGTATTAAGCGGGGGAGAATTAGATCGAT |
| | | GGGAAAAAATTCGGTTAAGGCCAGGGGGAAAGAAAAAATATAAATT |
| | | AAAACATATAGTATGGGCAAGCAGGGAGCTAGAACGATTCGCAGTT |
| | | AATCCTGGCCTGTTAGAAACATCAGAAGGCTGTAGACAAATACTGG |
| | | GACAGCTACAACCATCCCTTCAGACAGGATCAGAAGAACTTAGATC |
| | | ATTATATAATACAGTAGCAACCCTCTATTGTGTGCATCAAAGGATA |
| | | GAGATAAAAGACACCAAGGAAGCTTTAGACAAGATAGAGGAAGAGC |
| | | AAAACAAAAGTAAGAAAAAAGCACAGCAAGCAGCAGCTGACACAGG |
| | | ACACAGCAATCAGGTCAGCCAAAATTACCCTATAGTGCAGAACATC |
| | | CAGGGGCAAATGGTACATCAGGCCATATCACCTAGAACTTTAAATG |
| | | CATGGGTAAAAGTAGTAGAAGAGAAGGCTTTCAGCCCAGAAGTGAT |
| | | ACCCATGTTTTCAGCATTATCAGAAGGAGCCACCCCACAAGATTTA |
| | | AACACCATGCTAAACACAGTGGGGGGACATCAAGCAGCCATGCAAA |
| | | TGTTAAAAGAGACCATCAATGAGGAAGCTGCAGAATGGGATAGAGT |
| | | GCATCCAGTGCATGCAGGGCCTATTGCACCAGGCCAGATGAGAGAA |
| | | CCAAGGGGAAGTGACATAGCAGGAACTACTAGTACCCTTCAGGAAC |
| | | AAATAGGATGGATGACACATAATCCACCTATCCCAGTAGGAGAAAT |
| | | CTATAAAAGATGGATAATCCTGGGATTAAATAAAATAGTAAGAATG |
| | | TATAGCCCTACCAGCATTCTGGACATAAGACAAGGACCAAAGGAAC |
| | | CCTTTAGAGACTATGTAGACCGATTCTATAAAACTCTAAGAGCCGA |
| | | GCAAGCTTCACAAGAGGTAAAAAATTGGATGACAGAAACCTTGTTG |
| | | GTCCAAAATGCGAACCCAGATTGTAAGACTATTTTAAAAGCATTGG |
| | | GACCAGGAGCGACACTAGAAGAAATGATGACAGCATGTCAGGGAGT |
| | | GGGGGGACCCGGCCATAAAGCAAGAGTTTTGGCTGAAGCAATGAGC |
| | | CAAGTAACAAATCCAGCTACCATAATGATACAGAAAGGCAATTTTA |
| | | GGAACCAAAGAAAGACTGTTAAGTGTTTCAATTGTGGCAAAGAAGG |
| | | GCACATAGCCAAAAATTGCAGGGCCCCTAGGAAAAAGGGCTGTTGG |
| | | AAATGTGGAAAGGAAGGACACCAAATGAAAGATTGTACTGAGAGAC |
| | | AGGCTAATTTTTTAGGGAAGATCTGGCCTTCCCACAAGGGAAGGCC |
| | | AGGGAATTTTCTTCAGAGCAGACCAGAGCCAACAGCCCCACCAGAA |
| | | GAGAGCTTCAGGTTTGGGGAAGAGACAACAACTCCCTCTCAGAAGC |
| | | AGGAGCCGATAGACAAGGAACTGTATCCTTTAGCTTCCCTCAGATC |
| | | ACTCTTTGGCAGCGACCCCTCGTCACAATAA |
| 18 | HIV Pol | ATGAATTTGCCAGGAAGATGGAAACCAAAAATGATAGGGGGAATTG |
| | | GAGGTTTTATCAAAGTAGGACAGTATGATCAGATACTCATAGAAAT |
| | | CTGCGGACATAAAGCTATAGGTACAGTATTAGTAGGACCTACACCT |
| | | GTCAACATAATTGGAAGAAATCTGTTGACTCAGATTGGCTGCACTT |
| | | TAAATTTTCCCATTAGTCCTATTGAGACTGTACCAGTAAAATTAAA |
| | | GCCAGGAATGGATGGCCCAAAAGTTAAACAATGGCCATTGACAGAA |
| | | GAAAAAATAAAAGCATTAGTAGAAATTTGTACAGAAATGGAAAAGG |
| | | AAGGAAAAATTTCAAAAATTGGGCCTGAAAATCCATACAATACTCC |
| | | AGTATTTGCCATAAAGAAAAAAGACAGTACTAAATGGAGAAAATTA |
| | | GTAGATTTCAGAGAACTTAATAAGAGAACTCAAGATTTCTGGGAAG |
| | | TTCAATTAGGAATACCACATCCTGCAGGGTTAAAACAGAAAAAATC |
| | | AGTAACAGTACTGGATGTGGGCGATGCATATTTTTCAGTTCCCTTA |
| | | GATAAAGACTTCAGGAAGTATACTGCATTTACCATACCTAGTATAA |
| | | ACAATGAGACACCAGGGATTAGATATCAGTACAATGTGCTTCCACA |
| | | GGGATGGAAAGGATCACCAGCAATATTCCAGTGTAGCATGACAAAA |
| | | ATCTTAGAGCCTTTTAGAAAACAAAATCCAGACATAGTCATCTATC |
| | | AATACATGGATGATTTGTATGTAGGATCTGACTTAGAAATAGGGCA |
| | | GCATAGAACAAAAATAGAGGAACTGAGACAACATCTGTTGAGGTGG |
| | | GGATTTACCACACCAGACAAAAAACATCAGAAAGAACCTCCATTCC |
| | | TTTGGATGGGTTATGAACTCCATCCTGATAAATGGACAGTACAGCC |
| | | TATAGTGCTGCCAGAAAAGGACAGCTGGACTGTCAATGACATACAG |
| | | AAATTAGTGGGAAAATTGAATTGGGCAAGTCAGATTTATGCAGGGA |
| | | TTAAAGTAAGGCAATTATGTAAACTTCTTAGGGGAACCAAAGCACT |
| | | AACAGAAGTAGTACCACTAACAGAAGAAGCAGAGCTAGAACTGGCA |
| | | GAAAACAGGGAGATTCTAAAAGAACCGGTACATGGAGTGTATTATG |
| | | ACCCATCAAAAGACTTAATAGCAGAAATACAGAAGCAGGGGCAAGG |
| | | CCAATGGACATATCAAATTTATCAAGAGCCATTTAAAAATCTGAAA |
| | | ACAGGAAAATATGCAAGAATGAAGGGTGCCCACACTAATGATGTGA |
| | | AACAATTAACAGAGGCAGTACAAAAAATAGCCACAGAAAGCATAGT |
| | | AATATGGGGAAAGACTCCTAAATTTAAATTACCCATACAAAAGGAA |
| | | ACATGGGAAGCATGGTGGACAGAGTATTGGCAAGCCACCTGGATTC |
| | | CTGAGTGGGAGTTTGTCAATACCCCTCCCTTAGTGAAGTTATGGTA |
| | | CCAGTTAGAGAAAGAACCCATAATAGGAGCAGAAACTTTCTATGTA |
| | | GATGGGGCAGCCAATAGGGAAACTAAATTAGGAAAAGCAGGATATG |
| | | TAACTGACAGAGGAAGACAAAAAGTTGTCCCCCTAACGGACACAAC |
| | | AAATCAGAAGACTGAGTTACAAGCAATTCATCTAGCTTTGCAGGAT |
| | | TCGGGATTAGAAGTAAACATAGTGACAGACTCACAATATGCATTGG |
| | | GAATCATTCAAGCACAACCAGATAAGAGTGAATCAGAGTTAGTCAG |
| | | TCAAATAATAGAGCAGTTAATAAAAAAGGAAAAAGTCTACCTGGCA |
| | | TGGGTACCAGCACACAAAGGAATTGGAGGAAATGAACAAGTAGATG |
| | | GGTTGGTCAGTGCTGGAATCAGGAAAGTACTA |
| 19 | HIV Int | TTTTTAGATGGAATAGATAAGGCCCAAGAAGAACATGAGAAATATC |
| | | ACAGTAATTGGAGAGCAATGGCTAGTGATTTTAACCTACCACCTGT |
| | | AGTAGCAAAAGAAATAGTAGCCAGCTGTGATAAATGTCAGCTAAAA |
| | | GGGGAAGCCATGCATGGACAAGTAGACTGTAGCCCAGGAATATGGC |
| | | AGCTAGATTGTACACATTTAGAAGGAAAAGTTATCTTGGTAGCAGT |
| | | TCATGTAGCCAGTGGATATATAGAAGCAGAAGTAATTCCAGCAGAG |
| | | ACAGGGCAAGAAACAGCATACTTCCTCTTAAAATTAGCAGGAAGAT |
| | | GGCCAGTAAAAACAGTACATACAGACAATGGCAGCAATTTCACCAG |
| | | TACTACAGTTAAGGCCGCCTGTTGGTGGGCGGGGATCAAGCAGGAA |
| | | TTTGGCATTCCCTACAATCCCCAAAGTCAAGGAGTAATAGAATCTA |
| | | TGAATAAAGAATTAAAGAAAATTATAGGACAGGTAAGAGATCAGGC |
| | | TGAACATCTTAAGACAGCAGTACAAATGGCAGTATTCATCCACAAT |
| | | TTTAAAAGAAAAGGGGGGATTGGGGGGTACAGTGCAGGGGAAAGAA |
| | | TAGTAGACATAATAGCAACAGACATACAAACTAAAGAATTACAAAA |
| | | ACAAATTACAAAAATTCAAAATTTTCGGGTTTATTACAGGGACAGC |
| | | AGAGATCCAGTTTGGAAAGGACCAGCAAAGCTCCTCTGGAAAGGTG |
| | | AAGGGGCAGTAGTAATACAAGATAATAGTGACATAAAAGTAGTGCC |
| | | AAGAAGAAAAGCAAAGATCATCAGGGATTATGGAAAACAGATGGCA |
| | | GGTGATGATTGTGTGGCAAGTAGACAGGATGAGGATTAA |
| 20 | HIV RRE | AGGAGCTTTGTTCCTTGGGTTCTTGGGAGCAGCAGGAAGCACTATG |
| | | GGCGCAGCGTCAATGACGCTGACGGTACAGGCCAGACAATTATTGT |
| | | CTGGTATAGTGCAGCAGCAGAACAATTTGCTGAGGGCTATTGAGGC |
| | | GCAACAGCATCTGTTGCAACTCACAGTCTGGGGCATCAAGCAGCTC |
| | | CAGGCAAGAATCCTGGCTGTGGAAAGATACCTAAAGGATCAACAGC |
| | | TCCT |
| 21 | HIV Rev | ATGGCAGGAAGAAGCGGAGACAGCGACGAAGAACTCCTCAAGGCAG |
| | | TCAGACTCATCAAGTTTCTCTATCAAAGCAACCCACCTCCCAATCC |
| | | CGAGGGGACCCGACAGGCCCGAAGGAATAGAAGAAGAAGGTGGAGA |
| | | GAGAGACAGAGACAGATCCATTCGATTAGTGAACGGATCCTTAGCA |
| | | CTTATCTGGGACGATCTGCGGAGCCTGTGCCTCTTCAGCTACCACC |
| | | GCTTGAGAGACTTACTCTTGATTGTAACGAGGATTGTGGAACTTCT |
| | | GGGACGCAGGGGGTGGGAAGCCCTCAAATATTGGTGGAATCTCCTA |
| | | CAATATTGGAGTCAGGAGCTAAAGAATAG |
| 22 | CMV | ACATTGATTATTGACTAGTTATTAATAGTAATCAATTACGGGGTCA |
| | Promoter | TTAGTTCATAGCCCATATATGGAGTTCCGCGTTACATAACTTACGG |
| | | TAAATGGCCCGCCTGGCTGACCGCCCAACGACCCCCGCCCATTGAC |
| | | GTCAATAATGACGTATGTTCCCATAGTAACGCCAATAGGGACTTTC |
| | | CATTGACGTCAATGGGTGGAGTATTTACGGTAAACTGCCCACTTGG |
| | | CAGTACATCAAGTGTATCATATGCCAAGTACGCCCCCTATTGACGT |
| | | CAATGACGGTAAATGGCCCGCCTGGCATTATGCCCAGTACATGACC |
| | | TTATGGGACTTTCCTACTTGGCAGTACATCTACGTATTAGTCATCG |
| | | CTATTACCATGGTGATGCGGTTTTGGCAGTACATCAATGGGCGTGG |
| | | ATAGCGGTTTGACTCACGGGGATTTCCAAGTCTCCACCCCATTGAC |
| | | GTCAATGGGAGTTTGTTTTGGCACCAAAATCAACGGGACTTTCCAA |
| | | AATGTCGTAACAACTCCGCCCCATTGACGCAAATGGGCGGTAGGCG |
| | | TGTACGGTGGGAGGTCTATATAAGC |
| 23 | VSV-G/DNA | GAATTCATGAAGTGCCTTTTGTACTTAGCCTTTTTATTCATTGGGG |
| | fragment | TGAATTGCAAGTTCACCATAGTTTTTCCACACAACCAAAAAGGAAA |
| | containing | CTGGAAAAATGTTCCTTCTAATTACCATTATTGCCCGTCAAGCTCA |
| | VSV-G/ | GATTTAAATTGGCATAATGACTTAATAGGCACAGCCTTACAAGTCA |
| | Envelope | AAATGCCCAAGAGTCACAAGGCTATTCAAGCAGACGGTTGGATGTG |
| | Glycoprotein | TCATGCTTCCAAATGGGTCACTACTTGTGATTTCCGCTGGTATGGA |
| | | CCGAAGTATATAACACATTCCATCCGATCCTTCACTCCATCTGTAG |
| | | AACAATGCAAGGAAAGCATTGAACAAACGAAACAAGGAACTTGGCT |
| | | GAATCCAGGCTTCCCTCCTCAAAGTTGTGGATATGCAACTGTGACG |
| | | GATGCCGAAGCAGTGATTGTCCAGGTGACTCCTCACCATGTGCTGG |
| | | TTGATGAATACACAGGAGAATGGGTTGATTCACAGTTCATCAACGG |
| | | AAAATGCAGCAATTACATATGCCCCACTGTCCATAACTCTACAACC |
| | | TGGCATTCTGACTATAAGGTCAAAGGGCTATGTGATTCTAACCTCA |
| | | TTTCCATGGACATCACCTTCTTCTCAGAGGACGGAGAGCTATCATC |
| | | CCTGGGAAAGGAGGGCACAGGGTTCAGAAGTAACTACTTTGCTTAT |
| | | GAAACTGGAGGCAAGGCCTGCAAAATGCAATACTGCAAGCATTGGG |
| | | GAGTCAGACTCCCATCAGGTGTCTGGTTCGAGATGGCTGATAAGGA |
| | | TCTCTTTGCTGCAGCCAGATTCCCTGAATGCCCAGAAGGGTCAAGT |
| | | ATCTCTGCTCCATCTCAGACCTCAGTGGATGTAAGTCTAATTCAGG |
| | | ACGTTGAGAGGATCTTGGATTATTCCCTCTGCCAAGAAACCTGGAG |
| | | CAAAATCAGAGCGGGTCTTCCAATCTCTCCAGTGGATCTCAGCTAT |
| | | CTTGCTCCTAAAAACCCAGGAACCGGTCCTGCTTTCACCATAATCA |
| | | ATGGTACCCTAAAATACTTTGAGACCAGATACATCAGAGTCGATAT |
| | | TGCTGCTCCAATCCTCTCAAGAATGGTCGGAATGATCAGTGGAACT |
| | | ACCACAGAAAGGGAACTGTGGGATGACTGGGCACCATATGAAGACG |
| | | TGGAAATTGGACCCAATGGAGTTCTGAGGACCAGTTCAGGATATAA |
| | | GTTTCCTTTATACATGATTGGACATGGTATGTTGGACTCCGATCTT |
| | | CATCTTAGCTCAAAGGCTCAGGTGTTCGAACATCCTCACATTCAAG |
| | | ACGCTGCTTCGCAACTTCCTGATGATGAGAGTTTATTTTTTGGTGA |
| | | TACTGGGCTATCCAAAAATCCAATCGAGCTTGTAGAAGGTTGGTTC |
| | | AGTAGTTGGAAAAGCTCTATTGCCTCTTTTTTCTTTATCATAGGGT |
| | | TAATCATTGGACTATTCTTGGTTCTCCGAGTTGGTATCCATCTTTG |
| | | CATTAAATTAAAGCACACCAAGAAAAGACAGATTTATACAGACATA |
| | | GAGATGAGAATTC |
| 24 | CAG enhancer | TAGTTATTAATAGTAATCAATTACGGGGTCATTAGTTCATAGCCCA |
| | | TATATGGAGTTCCGCGTTACATAACTTACGGTAAATGGCCCGCCTG |
| | | GCTGACCGCCCAACGACCCCCGCCCATTGACGTCAATAATGACGTA |
| | | TGTTCCCATAGTAACGCCAATAGGGACTTTCCATTGACGTCAATGG |
| | | GTGGACTATTTACGGTAAACTGCCCACTTGGCAGTACATCAAGTGT |
| | | ATCATATGCCAAGTACGCCCCCTATTGACGTCAATGACGGTAAATG |
| | | GCCCGCCTGGCATTATGCCCAGTACATGACCTTATGGGACTTTCCT |
| | | ACTTGGCAGTACATCTACGTATTAGTCATC |
| 25 | Chicken beta | GGAGTCGCTGCGTTGCCTTCGCCCCGTGCCCCGCTCCGCGCCGCCT |
| | actin intron | CGCGCCGCCCGCCCCGGCTCTGACTGACCGCGTTACTCCCACAGGT |
| | | GAGCGGGCGGGACGGCCCTTCTCCTCCGGGCTGTAATTAGCGCTTG |
| | | GTTTAATGACGGCTCGTTTCTTTTCTGTGGCTGCGTGAAAGCCTTA |
| | | AAGGGCTCCGGGAGGGCCCTTTGTGCGGGGGGGAGCGGCTCGGGGG |
| | | GTGCGTGCGTGTGTGTGTGCGTGGGGAGCGCCGCGTGCGGCCCGCG |
| | | CTGCCCGGCGGCTGTGAGCGCTGCGGGCGCGGCGCGGGGCTTTGTG |
| | | CGCTCCGCGTGTGCGCGAGGGGAGCGCGGCCGGGGGCGGTGCCCCG |
| | | CGGTGCGGGGGGGCTGCGAGGGGAACAAAGGCTGCGTGCGGGGTGT |
| | | GTGCGTGGGGGGGTGAGCAGGGGGTGTGGGCGCGGCGGTCGGGCTG |
| | | TAACCCCCCCCTGCACCCCCCTCCCCGAGTTGCTGAGCACGGCCCG |
| | | GCTTCGGGTGCGGGGCTCCGTGCGGGGCGTGGCGCGGGGCTCGCCG |
| | | TGCCGGGCGGGGGGTGGCGGCAGGTGGGGGTGCCGGGCGGGGCGGG |
| | | GCCGCCTCGGGCCGGGGAGGGCTCGGGGGAGGGGCGCGGCGGCCCC |
| | | GGAGCGCCGGCGGCTGTCGAGGCGCGGCGAGCCGCAGCCATTGCCT |
| | | TTTATGGTAATCGTGCGAGAGGGCGCAGGGACTTCCTTTGTCCCAA |
| | | ATCTGGCGGAGCCGAAATCTGGGAGGCGCCGCCGCACCCCCTCTAG |
| | | CGGGCGCGGGCGAAGCGGTGCGGCGCCGGCAGGAAGGAAATGGGCG |
| | | GGGAGGGCCTTCGTGCGTCGCCGCGCCGCCGTCCCCTTCTCCATCT |
| | | CCAGCCTCGGGGCTGCCGCAGGGGGACGGCTGCCTTCGGGGGGGAC |
| | | GGGGCAGGGCGGGGTTCGGCTTCTGGCGTGTGACCGGCGG |
| 26 | Rabbit beta | AGATCTTTTTCCCTCTGCCAAAAATTATGGGGACATCATGAAGCCC |
| | globin poly A | CTTGAGCATCTGACTTCTGGCTAATAAAGGAAATTTATTTTCATTG |
| | | CAATAGTGTGTTGGAATTTTTTGTGTCTCTCACTCGGAAGGACATA |
| | | TGGGAGGGCAAATCATTTAAAACATCAGAATGAGTATTTGGTTTAG |
| | | AGTTTGGCAACATATGCCATATGCTGGCTGCCATGAACAAAGGTGG |
| | | CTATAAAGAGGTCATCAGTATATGAAACAGCCCCCTGCTGTCCATT |
| | | CCTTATTCCATAGAAAAGCCTTGACTTGAGGTTAGATTTTTTTTAT |
| | | ATTTTGTTTTGTGTTATTTTTTTCTTTAACATCCCTAAAATTTTCC |
| | | TTACATGTTTTACTAGCCAGATTTTTCCTCCTCTCCTGACTACTCC |
| | | CAGTCATAGCTGTCCCTCTTCTCTTATGAAGATC |
| 27 | Beta globin | GTGAGTTTGGGGACCCTTGATTGTTCTTTCTTTTTCGCTATTGTAA |
| | intron | AATTCATGTTATATGGAGGGGGCAAAGTTTTCAGGGTGTTGTTTAG |
| | | AATGGGAAGATGTCCCTTGTATCACCATGGACCCTCATGATAATTT |
| | | TGTTTCTTTCACTTTCTACTCTGTTGACAACCATTGTCTCCTCTTA |
| | | TTTTCTTTTCATTTTCTGTAACTTTTTCGTTAAACTTTAGCTTGCA |
| | | TTTGTAACGAATTTTTAAATTCACTTTTGTTTATTTGTCAGATTGT |
| | | AAGTACTTTCTCTAATCACTTTTTTTTCAAGGCAATCAGGGTATAT |
| | | TATATTGTACTTCAGCACAGTTTTAGAGAACAATTGTTATAATTAA |
| | | ATGATAAGGTAGAATATTTCTGCATATAAATTCTGGCTGGCGTGGA |
| | | AATATTCTTATTGGTAGAAACAACTACACCCTGGTCATCATCCTGC |
| | | CTTTCTCTTTATGGTTACAATGATATACACTGTTTGAGATGAGGAT |
| | | AAAATACTCTGAGTCCAAACCGGGCCCCTCTGCTAACCATGTTCAT |
| | | GCCTTCTTCTCTTTCCTACAG |
| 28 | Primer | TAAGCAGAATTCATGAATTTGCCAGGAAGAT |
| 29 | Primer | CCATACAATGAATGGACACTAGGCGGCCGCACGAAT |
| 30 | Gag, Pol, | GAATTCATGAATTTGCCAGGAAGATGGAAACCAAAAATGATAGGGG |
| | Integrase | GAATTGGAGGTTTTATCAAAGTAAGACAGTATGATCAGATACTCAT |
| | fragment | AGAAATCTGCGGACATAAAGCTATAGGTACAGTATTAGTAGGACCT |
| | | ACACCTGTCAACATAATTGGAAGAAATCTGTTGACTCAGATTGGCT |
| | | GCACTTTAAATTTTCCCATTAGTCCTATTGAGACTGTACCAGTAAA |
| | | ATTAAAGCCAGGAATGGATGGCCCAAAAGTTAAACAATGGCCATTG |
| | | ACAGAAGAAAAAATAAAAGCATTAGTAGAAATTTGTACAGAAATGG |
| | | AAAAGGAAGGAAAAATTTCAAAAATTGGGCCTGAAAATCCATACAA |
| | | TACTCCAGTATTTGCCATAAAGAAAAAAGACAGTACTAAATGGAGA |
| | | AAATTAGTAGATTTCAGAGAACTTAATAAGAGAACTCAAGATTTCT |
| | | GGGAAGTTCAATTAGGAATACCACATCCTGCAGGGTTAAAACAGAA |
| | | AAAATCAGTAACAGTACTGGATGTGGGCGATGCATATTTTTCAGTT |
| | | CCCTTAGATAAAGACTTCAGGAAGTATACTGCATTTACCATACCTA |
| | | GTATAAACAATGAGACACCAGGGATTAGATATCAGTACAATGTGCT |
| | | TCCACAGGGATGGAAAGGATCACCAGCAATATTCCAGTGTAGCATG |
| | | ACAAAAATCTTAGAGCCTTTTAGAAAACAAAATCCAGACATAGTCA |
| | | TCTATCAATACATGGATGATTTGTATGTAGGATCTGACTTAGAAAT |
| | | AGGGCAGCATAGAACAAAAATAGAGGAACTGAGACAACATCTGTTG |
| | | AGGTGGGGATTTACCACACCAGACAAAAAACATCAGAAAGAACCTC |
| | | CATTCCTTTGGATGGGTTATGAACTCCATCCTGATAAATGGACAGT |
| | | ACAGCCTATAGTGCTGCCAGAAAAGGACAGCTGGACTGTCAATGAC |
| | | ATACAGAAATTAGTGGGAAAATTGAATTGGGCAAGTCAGATTTATG |
| | | CAGGGATTAAAGTAAGGCAATTATGTAAACTTCTTAGGGGAACCAA |
| | | AGCACTAACAGAAGTAGTACCACTAACAGAAGAAGCAGAGCTAGAA |
| | | CTGGCAGAAAACAGGGAGATTCTAAAAGAACCGGTACATGGAGTGT |
| | | ATTATGACCCATCAAAAGACTTAATAGCAGAAATACAGAAGCAGGG |
| | | GCAAGGCCAATGGACATATCAAATTTATCAAGAGCCATTTAAAAAT |
| | | CTGAAAACAGGAAAGTATGCAAGAATGAAGGGTGCCCACACTAATG |
| | | ATGTGAAACAATTAACAGAGGCAGTACAAAAAATAGCCACAGAAAG |
| | | CATAGTAATATGGGGAAAGACTCCTAAATTTAAATTACCCATACAA |
| | | AAGGAAACATGGGAAGCATGGTGGACAGAGTATTGGCAAGCCACCT |
| | | GGATTCCTGAGTGGGAGTTTGTCAATACCCCTCCCTTAGTGAAGTT |
| | | ATGGTACCAGTTAGAGAAAGAACCCATAATAGGAGCAGAAACTTTC |
| | | TATGTAGATGGGGCAGCCAATAGGGAAACTAAATTAGGAAAAGCAG |
| | | GATATGTAACTGACAGAGGAAGACAAAAAGTTGTCCCCCTAACGGA |
| | | CACAACAAATCAGAAGACTGAGTTACAAGCAATTCATCTAGCTTTG |
| | | CAGGATTCGGGATTAGAAGTAAACATAGTGACAGACTCACAATATG |
| | | CATTGGGAATCATTCAAGCACAACCAGATAAGAGTGAATCAGAGTT |
| | | AGTCAGTCAAATAATAGAGCAGTTAATAAAAAAGGAAAAAGTCTAC |
| | | CTGGCATGGGTACCAGCACACAAAGGAATTGGAGGAAATGAACAAG |
| | | TAGATAAATTGGTCAGTGCTGGAATCAGGAAAGTACTATTTTTAGA |
| | | TGGAATAGATAAGGCCCAAGAAGAACATGAGAAATATCACAGTAAT |
| | | TGGAGAGCAATGGCTAGTGATTTTAACCTACCACCTGTAGTAGCAA |
| | | AAGAAATAGTAGCCAGCTGTGATAAATGTCAGCTAAAAGGGGAAGC |
| | | CATGCATGGACAAGTAGACTGTAGCCCAGGAATATGGCAGCTAGAT |
| | | TGTACACATTTAGAAGGAAAAGTTATCTTGGTAGCAGTTCATGTAG |
| | | CCAGTGGATATATAGAAGCAGAAGTAATTCCAGCAGAGACAGGGCA |
| | | AGAAACAGCATACTTCCTCTTAAAATTAGCAGGAAGATGGCCAGTA |
| | | AAAACAGTACATACAGACAATGGCAGCAATTTCACCAGTACTACAG |
| | | TTAAGGCCGCCTGTTGGTGGGCGGGGATCAAGCAGGAATTTGGCAT |
| | | TCCCTACAATCCCCAAAGTCAAGGAGTAATAGAATCTATGAATAAA |
| | | GAATTAAAGAAAATTATAGGACAGGTAAGAGATCAGGCTGAACATC |
| | | TTAAGACAGCAGTACAAATGGCAGTATTCATCCACAATTTTAAAAG |
| | | AAAAGGGGGGATTGGGGGGTACAGTGCAGGGGAAAGAATAGTAGAC |
| | | ATAATAGCAACAGACATACAAACTAAAGAATTACAAAAACAAATTA |
| | | CAAAAATTCAAAATTTTCGGGTTTATTACAGGGACAGCAGAGATCC |
| | | AGTTTGGAAAGGACCAGCAAAGCTCCTCTGGAAAGGTGAAGGGGCA |
| | | GTAGTAATACAAGATAATAGTGACATAAAAGTAGTGCCAAGAAGAA |
| | | AAGCAAAGATCATCAGGGATTATGGAAAACAGATGGCAGGTGATGA |
| | | TTGTGTGGCAAGTAGACAGGATGAGGATTAA |
| 31 | DNA | TCTAGAATGGCAGGAAGAAGCGGAGACAGCGACGAAGAGCTCATCA |
| | Fragment | GAACAGTCAGACTCATCAAGCTTCTCTATCAAAGCAACCCACCTCC |
| | containing | CAATCCCGAGGGGACCCGACAGGCCCGAAGGAATAGAAGAAGAAGG |
| | Rev, RRE and | TGGAGAGAGAGACAGAGACAGATCCATTCGATTAGTGAACGGATCC |
| | rabbit beta | TTGGCACTTATCTGGGACGATCTGCGGAGCCTGTGCCTCTTCAGCT |
| | globin poly A | ACCACCGCTTGAGAGACTTACTCTTGATTGTAACGAGGATTGTGGA |
| | | ACTTCTGGGACGCAGGGGGTGGGAAGCCCTCAAATATTGGTGGAAT |
| | | CTCCTACAATATTGGAGTCAGGAGCTAAAGAATAGAGGAGCTTTGT |
| | | TCCTTGGGTTCTTGGGAGCAGCAGGAAGCACTATGGGCGCAGCGTC |
| | | AATGACGCTGACGGTACAGGCCAGACAATTATTGTCTGGTATAGTG |
| | | CAGCAGCAGAACAATTTGCTGAGGGCTATTGAGGCGCAACAGCATC |
| | | TGTTGCAACTCACAGTCTGGGGCATCAAGCAGCTCCAGGCAAGAAT |
| | | CCTGGCTGTGGAAAGATACCTAAAGGATCAACAGCTCCTAGATCTT |
| | | TTTCCCTCTGCCAAAAATTATGGGGACATCATGAAGCCCCTTGAGC |
| | | ATCTGACTTCTGGCTAATAAAGGAAATTTATTTTCATTGCAATAGT |
| | | GTGTTGGAATTTTTTGTGTCTCTCACTCGGAAGGACATATGGGAGG |
| | | GCAAATCATTTAAAACATCAGAATGAGTATTTGGTTTAGAGTTTGG |
| | | CAACATATGCCATATGCTGGCTGCCATGAACAAAGGTGGCTATAAA |
| | | GAGGTCATCAGTATATGAAACAGCCCCCTGCTGTCCATTCCTTATT |
| | | CCATAGAAAAGCCTTGACTTGAGGTTAGATTTTTTTTATATTTTGT |
| | | TTTGTGTTATTTTTTTCTTTAACATCCCTAAAATTTTCCTTACATG |
| | | TTTTACTAGCCAGATTTTTCCTCCTCTCCTGACTACTCCCAGTCAT |
| | | AGCTGTCCCTCTTCTCTTATGAAGATCCCTCGACCTGCAGCCCAAG |
| | | CTTGGCGTAATCATGGTCATAGCTGTTTCCTGTGTGAAATTGTTAT |
| | | CCGCTCACAATTCCACACAACATACGAGCCGGAAGCATAAAGTGTA |
| | | AAGCCTGGGGTGCCTAATGAGTGAGCTAACTCACATTAATTGCGTT |
| | | GCGCTCACTGCCCGCTTTCCAGTCGGGAAACCTGTCGTGCCAGCGG |
| | | ATCCGCATCTCAATTAGTCAGCAACCATAGTCCCGCCCCTAACTCC |
| | | GCCCATCCCGCCCCTAACTCCGCCCAGTTCCGCCCATTCTCCGCCC |
| | | CATGGCTGACTAATTTTTTTTATTTATGCAGAGGCCGAGGCCGCCT |
| | | CGGCCTCTGAGCTATTCCAGAAGTAGTGAGGAGGCTTTTTTGGAGG |
| | | CCTAGGCTTTTGCAAAAAGCTAACTTGTTTATTGCAGCTTATAATG |
| | | GTTACAAATAAAGCAATAGCATCACAAATTTCACAAATAAAGCATT |
| | | TTTTTCACTGCATTCTAGTTGTGGTTTGTCCAAACTCATCAATGTA |
| | | TCTTATCAGCGGCCGCCCCGGG |
| 32 | DNA | ACGCGTTAGTTATTAATAGTAATCAATTACGGGGTCATTAGTTCAT |
| | fragment | AGCCCATATATGGAGTTCCGCGTTACATAACTTACGGTAAATGGCC |
| | containing | CGCCTGGCTGACCGCCCAACGACCCCCGCCCATTGACGTCAATAAT |
| | the CAG | GACGTATGTTCCCATAGTAACGCCAATAGGGACTTTCCATTGACGT |
| | enhancer/ | CAATGGGTGGACTATTTACGGTAAACTGCCCACTTGGCAGTACATC |
| | promoter/ | AAGTGTATCATATGCCAAGTACGCCCCCTATTGACGTCAATGACGG |
| | intron | TAAATGGCCCGCCTGGCATTATGCCCAGTACATGACCTTATGGGAC |
| | sequence | TTTCCTACTTGGCAGTACATCTACGTATTAGTCATCGCTATTACCA |
| | | TGGGTCGAGGTGAGCCCCACGTTCTGCTTCACTCTCCCCATCTCCC |
| | | CCCCCTCCCCACCCCCAATTTTGTATTTATTTATTTTTTAATTATT |
| | | TTGTGCAGCGATGGGGGCGGGGGGGGGGGGGGCGCGCGCCAGGCGG |
| | | GGCGGGGCGGGGCGAGGGGCGGGGCGGGGCGAGGCGGAGAGGTGCG |
| | | GCGGCAGCCAATCAGAGCGGCGCGCTCCGAAAGTTTCCTTTTATGG |
| | | CGAGGCGGCGGCGGCGGCGGCCCTATAAAAAGCGAAGCGCGCGGCG |
| | | GGCGGGAGTCGCTGCGTTGCCTTCGCCCCGTGCCCCGCTCCGCGCC |
| | | GCCTCGCGCCGCCCGCCCCGGCTCTGACTGACCGCGTTACTCCCAC |
| | | AGGTGAGCGGGCGGGACGGCCCTTCTCCTCCGGGCTGTAATTAGCG |
| | | CTTGGTTTAATGACGGCTCGTTTCTTTTCTGTGGCTGCGTGAAAGC |
| | | CTTAAAGGGCTCCGGGAGGGCCCTTTGTGCGGGGGGGAGCGGCTCG |
| | | GGGGGTGCGTGCGTGTGTGTGTGCGTGGGGAGCGCCGCGTGCGGCC |
| | | CGCGCTGCCCGGCGGCTGTGAGCGCTGCGGGCGCGGCGCGGGGCTT |
| | | TGTGCGCTCCGCGTGTGCGCGAGGGGAGCGCGGCCGGGGGCGGTGC |
| | | CCCGCGGTGCGGGGGGGCTGCGAGGGGAACAAAGGCTGCGTGCGGG |
| | | GTGTGTGCGTGGGGGGGTGAGCAGGGGGTGTGGGCGCGGCGGTCGG |
| | | GCTGTAACCCCCCCCTGCACCCCCCTCCCCGAGTTGCTGAGCACGG |
| | | CCCGGCTTCGGGTGCGGGGCTCCGTGCGGGGCGTGGCGCGGGGCTC |
| | | GCCGTGCCGGGCGGGGGGTGGCGGCAGGTGGGGGTGCCGGGCGGGG |
| | | CGGGGCCGCCTCGGGCCGGGGAGGGCTCGGGGGAGGGGCGCGGCGG |
| | | CCCCGGAGCGCCGGCGGCTGTCGAGGCGCGGCGAGCCGCAGCCATT |
| | | GCCTTTTATGGTAATCGTGCGAGAGGGCGCAGGGACTTCCTTTGTC |
| | | CCAAATCTGGCGGAGCCGAAATCTGGGAGGCGCCGCCGCACCCCCT |
| | | CTAGCGGGCGCGGGCGAAGCGGTGCGGCGCCGGCAGGAAGGAAATG |
| | | GGCGGGGAGGGCCTTCGTGCGTCGCCGCGCCGCCGTCCCCTTCTCC |
| | | ATCTCCAGCCTCGGGGCTGCCGCAGGGGGACGGCTGCCTTCGGGGG |
| | | GGACGGGGCAGGGCGGGGTTCGGCTTCTGGCGTGTGACCGGCGGGA |
| | | ATTC |
| 33 | RSV promoter | CAATTGCGATGTACGGGCCAGATATACGCGTATCTGAGGGGACTAG |
| | and HIV Rev | GGTGTGTTTAGGCGAAAAGCGGGGCTTCGGTTGTACGCGGTTAGGA |
| | | GTCCCCTCAGGATATAGTAGTTTCGCTTTTGCATAGGGAGGGGGAA |
| | | ATGTAGTCTTATGCAATACACTTGTAGTCTTGCAACATGGTAACGA |
| | | TGAGTTAGCAACATGCCTTACAAGGAGAGAAAAAGCACCGTGCATG |
| | | CCGATTGGTGGAAGTAAGGTGGTACGATCGTGCCTTATTAGGAAGG |
| | | CAACAGACAGGTCTGACATGGATTGGACGAACCACTGAATTCCGCA |
| | | TTGCAGAGATAATTGTATTTAAGTGCCTAGCTCGATACAATAAACG |
| | | CCATTTGACCATTCACCACATTGGTGTGCACCTCCAAGCTCGAGCT |
| | | CGTTTAGTGAACCGTCAGATCGCCTGGAGACGCCATCCACGCTGTT |
| | | TTGACCTCCATAGAAGACACCGGGACCGATCCAGCCTCCCCTCGAA |
| | | GCTAGCGATTAGGCATCTCCTATGGCAGGAAGAAGCGGAGACAGCG |
| | | ACGAAGAACTCCTCAAGGCAGTCAGACTCATCAAGTTTCTCTATCA |
| | | AAGCAACCCACCTCCCAATCCCGAGGGGACCCGACAGGCCCGAAGG |
| | | AATAGAAGAAGAAGGTGGAGAGAGAGACAGAGACAGATCCATTCGA |
| | | TTAGTGAACGGATCCTTAGCACTTATCTGGGACGATCTGCGGAGCC |
| | | TGTGCCTCTTCAGCTACCACCGCTTGAGAGACTTACTCTTGATTGT |
| | | AACGAGGATTGTGGAACTTCTGGGACGCAGGGGGTGGGAAGCCCTC |
| | | AAATATTGGTGGAATCTCCTACAATATTGGAGTCAGGAGCTAAAGA |
| | | ATAGTCTAGA |
| 34 | Elongation | CCGGTGCCTAGAGAAGGTGGCGCGGGGTAAACTGGGAAAGTGATGT |
| | Factor-1 alpha | CGTGTACTGGCTCCGCCTTTTTCCCGAGGGTGGGGGAGAACCGTAT |
| | (EF1-alpha) | ATAAGTGCAGTAGTCGCCGTGAACGTTCTTTTTCGCAACGGGTTTG |
| | promoter | CCGCCAGAACACAGGTAAGTGCCGTGTGTGGTTCCCGCGGGCCTGG |
| | | CCTCTTTACGGGTTATGGCCCTTGCGTGCCTTGAATTACTTCCACG |
| | | CCCCTGGCTGCAGTACGTGATTCTTGATCCCGAGCTTCGGGTTGGA |
| | | AGTGGGTGGGAGAGTTCGAGGCCTTGCGCTTAAGGAGCCCCTTCGC |
| | | CTCGTGCTTGAGTTGAGGCCTGGCCTGGGCGCTGGGGCCGCCGCGT |
| | | GCGAATCTGGTGGCACCTTCGCGCCTGTCTCGCTGCTTTCGATAAG |
| | | TCTCTAGCCATTTAAAATTTTTGATGACCTGCTGCGACGCTTTTTT |
| | | TCTGGCAAGATAGTCTTGTAAATGCGGGCCAAGATCTGCACACTGG |
| | | TATTTCGGTTTTTGGGGCCGCGGGCGGCGACGGGGCCCGTGCGTCC |
| | | CAGCGCACATGTTCGGCGAGGCGGGGCCTGCGAGCGCGGCCACCGA |
| | | GAATCGGACGGGGGTAGTCTCAAGCTGGCCGGCCTGCTCTGGTGCC |
| | | TGGCCTCGCGCCGCCGTGTATCGCCCCGCCCTGGGCGGCAAGGCTG |
| | | GCCCGGTCGGCACCAGTTGCGTGAGCGGAAAGATGGCCGCTTCCCG |
| | | GCCCTGCTGCAGGGAGCTCAAAATGGAGGACGCGGCGCTCGGGAGA |
| | | GCGGGCGGGTGAGTCACCCACACAAAGGAAAAGGGCCTTTCCGTCC |
| | | TCAGCCGTCGCTTCATGTGACTCCACGGAGTACCGGGCGCCGTCCA |
| | | GGCACCTCGATTAGTTCTCGAGCTTTTGGAGTACGTCGTCTTTAGG |
| | | TTGGGGGGAGGGGTTTTATGCGATGGAGTTTCCCCACACTGAGTGG |
| | | GTGGAGACTGAAGTTAGGCCAGCTTGGCACTTGATGTAATTCTCCT |
| | | TGGAATTTGCCCTTTTTGAGTTTGGATCTTGGTTCATTCTCAAGCC |
| | | TCAGACAGTGGTTCAAAGTTTTTTTCTTCCATTTCAGGTGTCGTGA |
| 35 | Promoter; | GGGGTTGGGGTTGCGCCTTTTCCAAGGCAGCCCTGGGTTTGCGCAG |
| | PGK | GGACGCGGCTGCTCTGGGCGTGGTTCCGGGAAACGCAGCGGCGCCG |
| | | ACCCTGGGTCTCGCACATTCTTCACGTCCGTTCGCAGCGTCACCCG |
| | | GATCTTCGCCGCTACCCTTGTGGGCCCCCCGGCGACGCTTCCTGCT |
| | | CCGCCCCTAAGTCGGGAAGGTTCCTTGCGGTTCGCGGCGTGCCGGA |
| | | CGTGACAAACGGAAGCCGCACGTCTCACTAGTACCCTCGCAGACGG |
| | | ACAGCGCCAGGGAGCAATGGCAGCGCGCCGACCGCGATGGGCTGTG |
| | | GCCAATAGCGGCTGCTCAGCAGGGCGCGCCGAGAGCAGCGGCCGGG |
| | | AAGGGGCGGTGCGGGAGGCGGGGTGTGGGGCGGTAGTGTGGGCCCT |
| | | GTTCCTGCCCGCGCGGTGTTCCGCATTCTGCAAGCCTCCGGAGCGC |
| | | ACGTCGGCAGTCGGCTCCCTCGTTGACCGAATCACCGACCTCTCTC |
| | | CCCAG |
| 36 | Promoter; | GCGCCGGGTTTTGGCGCCTCCCGCGGGCGCCCCCCTCCTCACGGCG |
| | UbC | AGCGCTGCCACGTCAGACGAAGGGCGCAGGAGCGTTCCTGATCCTT |
| | | CCGCCCGGACGCTCAGGACAGCGGCCCGCTGCTCATAAGACTCGGC |
| | | CTTAGAACCCCAGTATCAGCAGAAGGACATTTTAGGACGGGACTTG |
| | | GGTGACTCTAGGGCACTGGTTTTCTTTCCAGAGAGCGGAACAGGCG |
| | | AGGAAAAGTAGTCCCTTCTCGGCGATTCTGCGGAGGGATCTCCGTG |
| | | GGGCGGTGAACGCCGATGATTATATAAGGACGCGCCGGGTGTGGCA |
| | | CAGCTAGTTCCGTCGCAGCCGGGATTTGGGTCGCGGTTCTTGTTTG |
| | | TGGATCGCTGTGATCGTCACTTGGTGAGTTGCGGGCTGCTGGGCTG |
| | | GCCGGGGCTTTCGTGGCCGCCGGGCCGCTCGGTGGGACGGAAGCGT |
| | | GTGGAGAGACCGCCAAGGGCTGTAGTCTGGGTCCGCGAGCAAGGTT |
| | | GCCCTGAACTGGGGGTTGGGGGGAGCGCACAAAATGGCGGCTGTTC |
| | | CCGAGTCTTGAATGGAAGACGCTTGTAAGGCGGGCTGTGAGGTCGT |
| | | TGAAACAAGGTGGGGGGCATGGTGGGCGGCAAGAACCCAAGGTCTT |
| | | GAGGCCTTCGCTAATGCGGGAAAGCTCTTATTCGGGTGAGATGGGC |
| | | TGGGGCACCATCTGGGGACCCTGACGTGAAGTTTGTCACTGACTGG |
| | | AGAACTCGGGTTTGTCGTCTGGTTGCGGGGGCGGCAGTTATGCGGT |
| | | GCCGTTGGGCAGTGCACCCGTACCTTTGGGAGCGCGCGCCTCGTCG |
| | | TGTCGTGACGTCACCCGTTCTGTTGGCTTATAATGCAGGGTGGGGC |
| | | CACCTGCCGGTAGGTGTGCGGTAGGCTTTTCTCCGTCGCAGGACGC |
| | | AGGGTTCGGGCCTAGGGTAGGCTCTCCTGAATCGACAGGCGCCGGA |
| | | CCTCTGGTGAGGGGAGGGATAAGTGAGGCGTCAGTTTCTTTGGTCG |
| | | GTTTTATGTACCTATCTTCTTAAGTAGCTGAAGCTCCGGTTTTGAA |
| | | CTATGCGCTCGGGGTTGGCGAGTGTGTTTTGTGAAGTTTTTTAGGC |
| | | ACCTTTTGAAATGTAATCATTTGGGTCAATATGTAATTTTCAGTGT |
| | | TAGACTAGTAAA |
| 37 | Poly A; SV40 | GTTTATTGCAGCTTATAATGGTTACAAATAAAGCAATAGCATCACA |
| | | AATTTCACAAATAAAGCATTTTTTTCACTGCATTCTAGTTGTGGTT |
| | | TGTCCAAACTCATCAATGTATCTTATCA |
| 38 | Poly A; bGH | GACTGTGCCTTCTAGTTGCCAGCCATCTGTTGTTTGCCCCTCCCCC |
| | | GTGCCTTCCTTGACCCTGGAAGGTGCCACTCCCACTGTCCTTTCCT |
| | | AATAAAATGAGGAAATTGCATCGCATTGTCTGAGTAGGTGTCATTC |
| | | TATTCTGGGGGGTGGGGTGGGGCAGGACAGCAAGGGGGAGGATTGG |
| | | GAAGACAATAGCAGGCATGCTGGGGATGCGGTGGGCTCTATGG |
| 39 | Envelope; | ATGAAACTCCCAACAGGAATGGTCATTTTATGTAGCCTAATAATAG |
| | RD114 | TTCGGGCAGGGTTTGACGACCCCCGCAAGGCTATCGCATTAGTACA |
| | | AAAACAACATGGTAAACCATGCGAATGCAGCGGAGGGCAGGTATCC |
| | | GAGGCCCCACCGAACTCCATCCAACAGGTAACTTGCCCAGGCAAGA |
| | | CGGCCTACTTAATGACCAACCAAAAATGGAAATGCAGAGTCACTCC |
| | | AAAAAATCTCACCCCTAGCGGGGGAGAACTCCAGAACTGCCCCTGT |
| | | AACACTTTCCAGGACTCGATGCACAGTTCTTGTTATACTGAATACC |
| | | GGCAATGCAGGGCGAATAATAAGACATACTACACGGCCACCTTGCT |
| | | TAAAATACGGTCTGGGAGCCTCAACGAGGTACAGATATTACAAAAC |
| | | CCCAATCAGCTCCTACAGTCCCCTTGTAGGGGCTCTATAAATCAGC |
| | | CCGTTTGCTGGAGTGCCACAGCCCCCATCCATATCTCCGATGGTGG |
| | | AGGACCCCTCGATACTAAGAGAGTGTGGACAGTCCAAAAAAGGCTA |
| | | GAACAAATTCATAAGGCTATGCATCCTGAACTTCAATACCACCCCT |
| | | TAGCCCTGCCCAAAGTCAGAGATGACCTTAGCCTTGATGCACGGAC |
| | | TTTTGATATCCTGAATACCACTTTTAGGTTACTCCAGATGTCCAAT |
| | | TTTAGCCTTGCCCAAGATTGTTGGCTCTGTTTAAAACTAGGTACCC |
| | | CTACCCCTCTTGCGATACCCACTCCCTCTTTAACCTACTCCCTAGC |
| | | AGACTCCCTAGCGAATGCCTCCTGTCAGATTATACCTCCCCTCTTG |
| | | GTTCAACCGATGCAGTTCTCCAACTCGTCCTGTTTATCTTCCCCTT |
| | | TCATTAACGATACGGAACAAATAGACTTAGGTGCAGTCACCTTTAC |
| | | TAACTGCACCTCTGTAGCCAATGTCAGTAGTCCTTTATGTGCCCTA |
| | | AACGGGTCAGTCTTCCTCTGTGGAAATAACATGGCATACACCTATT |
| | | TACCCCAAAACTGGACAGGACTTTGCGTCCAAGCCTCCCTCCTCCC |
| | | CGACATTGACATCATCCCGGGGGATGAGCCAGTCCCCATTCCTGCC |
| | | ATTGATCATTATATACATAGACCTAAACGAGCTGTACAGTTCATCC |
| | | CTTTACTAGCTGGACTGGGAATCACCGCAGCATTCACCACCGGAGC |
| | | TACAGGCCTAGGTGTCTCCGTCACCCAGTATACAAAATTATCCCAT |
| | | CAGTTAATATCTGATGTCCAAGTCTTATCCGGTACCATACAAGATT |
| | | TACAAGACCAGGTAGACTCGTTAGCTGAAGTAGTTCTCCAAAATAG |
| | | GAGGGGACTGGACCTACTAACGGCAGAACAAGGAGGAATTTGTTTA |
| | | GCCTTACAAGAAAAATGCTGTTTTTATGCTAACAAGTCAGGAATTG |
| | | TGAGAAACAAAATAAGAACCCTACAAGAAGAATTACAAAAACGCAG |
| | | GGAAAGCCTGGCATCCAACCCTCTCTGGACCGGGCTGCAGGGCTTT |
| | | CTTCCGTACCTCCTACCTCTCCTGGGACCCCTACTCACCCTCCTAC |
| | | TCATACTAACCATTGGGCCATGCGTTTTCAATCGATTGGTCCAATT |
| | | TGTTAAAGACAGGATCTCAGTGGTCCAGGCTCTGGTTTTGACTCAG |
| | | CAATATCACCAGCTAAAACCCATAGAGTACGAGCCATGA |
| 40 | Envelope; | ATGCTTCTCACCTCAAGCCCGCACCACCTTCGGCACCAGATGAGTC |
| | GALV | CTGGGAGCTGGAAAAGACTGATCATCCTCTTAAGCTGCGTATTCGG |
| | | AGACGGCAAAACGAGTCTGCAGAATAAGAACCCCCACCAGCCTGTG |
| | | ACCCTCACCTGGCAGGTACTGTCCCAAACTGGGGACGTTGTCTGGG |
| | | ACAAAAAGGCAGTCCAGCCCCTTTGGACTTGGTGGCCCTCTCTTAC |
| | | ACCTGATGTATGTGCCCTGGCGGCCGGTCTTGAGTCCTGGGATATC |
| | | CCGGGATCCGATGTATCGTCCTCTAAAAGAGTTAGACCTCCTGATT |
| | | CAGACTATACTGCCGCTTATAAGCAAATCACCTGGGGAGCCATAGG |
| | | GTGCAGCTACCCTCGGGCTAGGACCAGGATGGCAAATTCCCCCTTC |
| | | TACGTGTGTCCCCGAGCTGGCCGAACCCATTCAGAAGCTAGGAGGT |
| | | GTGGGGGGCTAGAATCCCTATACTGTAAAGAATGGAGTTGTGAGAC |
| | | CACGGGTACCGTTTATTGGCAACCCAAGTCCTCATGGGACCTCATA |
| | | ACTGTAAAATGGGACCAAAATGTGAAATGGGAGCAAAAATTTCAAA |
| | | AGTGTGAACAAACCGGCTGGTGTAACCCCCTCAAGATAGACTTCAC |
| | | AGAAAAAGGAAAACTCTCCAGAGATTGGATAACGGAAAAAACCTGG |
| | | GAATTAAGGTTCTATGTATATGGACACCCAGGCATACAGTTGACTA |
| | | TCCGCTTAGAGGTCACTAACATGCCGGTTGTGGCAGTGGGCCCAGA |
| | | CCCTGTCCTTGCGGAACAGGGACCTCCTAGCAAGCCCCTCACTCTC |
| | | CCTCTCTCCCCACGGAAAGCGCCGCCCACCCCTCTACCCCCGGCGG |
| | | CTAGTGAGCAAACCCCTGCGGTGCATGGAGAAACTGTTACCCTAAA |
| | | CTCTCCGCCTCCCACCAGTGGCGACCGACTCTTTGGCCTTGTGCAG |
| | | GGGGCCTTCCTAACCTTGAATGCTACCAACCCAGGGGCCACTAAGT |
| | | CTTGCTGGCTCTGTTTGGGCATGAGCCCCCCTTATTATGAAGGGAT |
| | | AGCCTCTTCAGGAGAGGTCGCTTATACCTCCAACCATACCCGATGC |
| | | CACTGGGGGGCCCAAGGAAAGCTTACCCTCACTGAGGTCTCCGGAC |
| | | TCGGGTCATGCATAGGGAAGGTGCCTCTTACCCATCAACATCTTTG |
| | | CAACCAGACCTTACCCATCAATTCCTCTAAAAACCATCAGTATCTG |
| | | CTCCCCTCAAACCATAGCTGGTGGGCCTGCAGCACTGGCCTCACCC |
| | | CCTGCCTCTCCACCTCAGTTTTTAATCAGTCTAAAGACTTCTGTGT |
| | | CCAGGTCCAGCTGATCCCCCGCATCTATTACCATTCTGAAGAAACC |
| | | TTGTTACAAGCCTATGACAAATCACCCCCCAGGTTTAAAAGAGAGC |
| | | CTGCCTCACTTACCCTAGCTGTCTTCCTGGGGTTAGGGATTGCGGC |
| | | AGGTATAGGTACTGGCTCAACCGCCCTAATTAAAGGGCCCATAGAC |
| | | CTCCAGCAAGGCCTAACCAGCCTCCAAATCGCCATTGACGCTGACC |
| | | TCCGGGCCCTTCAGGACTCAATCAGCAAGCTAGAGGACTCACTGAC |
| | | TTCCCTATCTGAGGTAGTACTCCAAAATAGGAGAGGCCTTGACTTA |
| | | CTATTCCTTAAAGAAGGAGGCCTCTGCGCGGCCCTAAAAGAAGAGT |
| | | GCTGTTTTTATGTAGACCACTCAGGTGCAGTACGAGACTCCATGAA |
| | | AAAACTTAAAGAAAGACTAGATAAAAGACAGTTAGAGCGCCAGAAA |
| | | AACCAAAACTGGTATGAAGGGTGGTTCAATAACTCCCCTTGGTTTA |
| | | CTACCCTACTATCAACCATCGCTGGGCCCCTATTGCTCCTCCTTTT |
| | | GTTACTCACTCTTGGGCCCTGCATCATCAATAAATTAATCCAATTC |
| | | ATCAATGATAGGATAAGTGCAGTCAAAATTTTAGTCCTTAGACAGA |
| | | AATATCAGACCCTAGATAACGAGGAAAACCTTTAA |
| 41 | Envelope; | ATGGTTCCGCAGGTTCTTTTGTTTGTACTCCTTCTGGGTTTTTCGT |
| | FUG | TGTGTTTCGGGAAGTTCCCCATTTACACGATACCAGACGAACTTGG |
| | | TCCCTGGAGCCCTATTGACATACACCATCTCAGCTGTCCAAATAAC |
| | | CTGGTTGTGGAGGATGAAGGATGTACCAACCTGTCCGAGTTCTCCT |
| | | ACATGGAACTCAAAGTGGGATACATCTCAGCCATCAAAGTGAACGG |
| | | GTTCACTTGCACAGGTGTTGTGACAGAGGCAGAGACCTACACCAAC |
| | | TTTGTTGGTTATGTCACAACCACATTCAAGAGAAAGCATTTCCGCC |
| | | CCACCCCAGACGCATGTAGAGCCGCGTATAACTGGAAGATGGCCGG |
| | | TGACCCCAGATATGAAGAGTCCCTACACAATCCATACCCCGACTAC |
| | | CACTGGCTTCGAACTGTAAGAACCACCAAAGAGTCCCTCATTATCA |
| | | TATCCCCAAGTGTGACAGATTTGGACCCATATGACAAATCCCTTCA |
| | | CTCAAGGGTCTTCCCTGGCGGAAAGTGCTCAGGAATAACGGTGTCC |
| | | TCTACCTACTGCTCAACTAACCATGATTACACCATTTGGATGCCCG |
| | | AGAATCCGAGACCAAGGACACCTTGTGACATTTTTACCAATAGCAG |
| | | AGGGAAGAGAGCATCCAACGGGAACAAGACTTGCGGCTTTGTGGAT |
| | | GAAAGAGGCCTGTATAAGTCTCTAAAAGGAGCATGCAGGCTCAAGT |
| | | TATGTGGAGTTCTTGGACTTAGACTTATGGATGGAACATGGGTCGC |
| | | GATGCAAACATCAGATGAGACCAAATGGTGCCCTCCAGATCAGTTG |
| | | GTGAATTTGCACGACTTTCGCTCAGACGAGATCGAGCATCTCGTTG |
| | | TGGAGGAGTTAGTTAAGAAAAGAGAGGAATGTCTGGATGCATTAGA |
| | | GTCCATCATGACCACCAAGTCAGTAAGTTTCAGACGTCTCAGTCAC |
| | | CTGAGAAAACTTGTCCCAGGGTTTGGAAAAGCATATACCATATTCA |
| | | ACAAAACCTTGATGGAGGCTGATGCTCACTACAAGTCAGTCCGGAC |
| | | CTGGAATGAGATCATCCCCTCAAAAGGGTGTTTGAAAGTTGGAGGA |
| | | AGGTGCCATCCTCATGTGAACGGGGTGTTTTTCAATGGTATAATAT |
| | | TAGGGCCTGACGACCATGTCCTAATCCCAGAGATGCAATCATCCCT |
| | | CCTCCAGCAACATATGGAGTTGTTGGAATCTTCAGTTATCCCCCTG |
| | | ATGCACCCCCTGGCAGACCCTTCTACAGTTTTCAAAGAAGGTGATG |
| | | AGGCTGAGGATTTTGTTGAAGTTCACCTCCCCGATGTGTACAAACA |
| | | GATCTCAGGGGTTGACCTGGGTCTCCCGAACTGGGGAAAGTATGTA |
| | | TTGATGACTGCAGGGGCCATGATTGGCCTGGTGTTGATATTTTCCC |
| | | TAATGACATGGTGCAGAGTTGGTATCCATCTTTGCATTAAATTAAA |
| | | GCACACCAAGAAAAGACAGATTTATACAGACATAGAGATGAACCGA |
| | | CTTGGAAAGTAA |
| 42 | Envelope; | ATGGGTCAGATTGTGACAATGTTTGAGGCTCTGCCTCACATCATCG |
| | LCMV | ATGAGGTGATCAACATTGTCATTATTGTGCTTATCGTGATCACGGG |
| | | TATCAAGGCTGTCTACAATTTTGCCACCTGTGGGATATTCGCATTG |
| | | ATCAGTTTCCTACTTCTGGCTGGCAGGTCCTGTGGCATGTACGGTC |
| | | TTAAGGGACCCGACATTTACAAAGGAGTTTACCAATTTAAGTCAGT |
| | | GGAGTTTGATATGTCACATCTGAACCTGACCATGCCCAACGCATGT |
| | | TCAGCCAACAACTCCCACCATTACATCAGTATGGGGACTTCTGGAC |
| | | TAGAATTGACCTTCACCAATGATTCCATCATCAGTCACAACTTTTG |
| | | CAATCTGACCTCTGCCTTCAACAAAAAGACCTTTGACCACACACTC |
| | | ATGAGTATAGTTTCGAGCCTACACCTCAGTATCAGAGGGAACTCCA |
| | | ACTATAAGGCAGTATCCTGCGACTTCAACAATGGCATAACCATCCA |
| | | ATACAACTTGACATTCTCAGATCGACAAAGTGCTCAGAGCCAGTGT |
| | | AGAACCTTCAGAGGTAGAGTCCTAGATATGTTTAGAACTGCCTTCG |
| | | GGGGGAAATACATGAGGAGTGGCTGGGGCTGGACAGGCTCAGATGG |
| | | CAAGACCACCTGGTGTAGCCAGACGAGTTACCAATACCTGATTATA |
| | | CAAAATAGAACCTGGGAAAACCACTGCACATATGCAGGTCCTTTTG |
| | | GGATGTCCAGGATTCTCCTTTCCCAAGAGAAGACTAAGTTCTTCAC |
| | | TAGGAGACTAGCGGGCACATTCACCTGGACTTTGTCAGACTCTTCA |
| | | GGGGTGGAGAATCCAGGTGGTTATTGCCTGACCAAATGGATGATTC |
| | | TTGCTGCAGAGCTTAAGTGTTTCGGGAACACAGCAGTTGCGAAATG |
| | | CAATGTAAATCATGATGCCGAATTCTGTGACATGCTGCGACTAATT |
| | | GACTACAACAAGGCTGCTTTGAGTAAGTTCAAAGAGGACGTAGAAT |
| | | CTGCCTTGCACTTATTCAAAACAACAGTGAATTCTTTGATTTCAGA |
| | | TCAACTACTGATGAGGAACCACTTGAGAGATCTGATGGGGGTGCCA |
| | | TATTGCAATTACTCAAAGTTTTGGTACCTAGAACATGCAAAGACCG |
| | | GCGAAACTAGTGTCCCCAAGTGCTGGCTTGTCACCAATGGTTCTTA |
| | | CTTAAATGAGACCCACTTCAGTGATCAAATCGAACAGGAAGCCGAT |
| | | AACATGATTACAGAGATGTTGAGGAAGGATTACATAAAGAGGCAGG |
| | | GGAGTACCCCCCTAGCATTGATGGACCTTCTGATGTTTTCCACATC |
| | | TGCATATCTAGTCAGCATCTTCCTGCACCTTGTCAAAATACCAACA |
| | | CACAGGCACATAAAAGGTGGCTCATGTCCAAAGCCACACCGATTAA |
| | | CCAACAAAGGAATTTGTAGTTGTGGTGCATTTAAGGTGCCTGGTGT |
| | | AAAAACCGTCTGGAAAAGACGCTGA |
| 43 | Envelope; | ATGAACACTCAAATCCTGGTTTTCGCCCTTGTGGCAGTCATCCCCA |
| | FPV | CAAATGCAGACAAAATTTGTCTTGGACATCATGCTGTATCAAATGG |
| | | CACCAAAGTAAACACACTCACTGAGAGAGGAGTAGAAGTTGTCAAT |
| | | GCAACGGAAACAGTGGAGCGGACAAACATCCCCAAAATTTGCTCAA |
| | | AAGGGAAAAGAACCACTGATCTTGGCCAATGCGGACTGTTAGGGAC |
| | | CATTACCGGACCACCTCAATGCGACCAATTTCTAGAATTTTCAGCT |
| | | GATCTAATAATCGAGAGACGAGAAGGAAATGATGTTTGTTACCCGG |
| | | GGAAGTTTGTTAATGAAGAGGCATTGCGACAAATCCTCAGAGGATC |
| | | AGGTGGGATTGACAAAGAAACAATGGGATTCACATATAGTGGAATA |
| | | AGGACCAACGGAACAACTAGTGCATGTAGAAGATCAGGGTCTTCAT |
| | | TCTATGCAGAAATGGAGTGGCTCCTGTCAAATACAGACAATGCTGC |
| | | TTTCCCACAAATGACAAAATCATACAAAAACACAAGGAGAGAATCA |
| | | GCTCTGATAGTCTGGGGAATCCACCATTCAGGATCAACCACCGAAC |
| | | AGACCAAACTATATGGGAGTGGAAATAAACTGATAACAGTCGGGAG |
| | | TTCCAAATATCATCAATCTTTTGTGCCGAGTCCAGGAACACGACCG |
| | | CAGATAAATGGCCAGTCCGGACGGATTGATTTTCATTGGTTGATCT |
| | | TGGATCCCAATGATACAGTTACTTTTAGTTTCAATGGGGCTTTCAT |
| | | AGCTCCAAATCGTGCCAGCTTCTTGAGGGGAAAGTCCATGGGGATC |
| | | CAGAGCGATGTGCAGGTTGATGCCAATTGCGAAGGGGAATGCTACC |
| | | ACAGTGGAGGGACTATAACAAGCAGATTGCCTTTTCAAAACATCAA |
| | | TAGCAGAGCAGTTGGCAAATGCCCAAGATATGTAAAACAGGAAAGT |
| | | TTATTATTGGCAACTGGGATGAAGAACGTTCCCGAACCTTCCAAAA |
| | | AAAGGAAAAAAAGAGGCCTGTTTGGCGCTATAGCAGGGTTTATTGA |
| | | AAATGGTTGGGAAGGTCTGGTCGACGGGTGGTACGGTTTCAGGCAT |
| | | CAGAATGCACAAGGAGAAGGAACTGCAGCAGACTACAAAAGCACCC |
| | | AATCGGCAATTGATCAGATAACCGGAAAGTTAAATAGACTCATTGA |
| | | GAAAACCAACCAGCAATTTGAGCTAATAGATAATGAATTCACTGAG |
| | | GTGGAAAAGCAGATTGGCAATTTAATTAACTGGACCAAAGACTCCA |
| | | TCACAGAAGTATGGTCTTACAATGCTGAACTTCTTGTGGCAATGGA |
| | | AAACCAGCACACTATTGATTTGGCTGATTCAGAGATGAACAAGCTG |
| | | TATGAGCGAGTGAGGAAACAATTAAGGGAAAATGCTGAAGAGGATG |
| | | GCACTGGTTGCTTTGAAATTTTTCATAAATGTGACGATGATTGTAT |
| | | GGCTAGTATAAGGAACAATACTTATGATCACAGCAAATACAGAGAA |
| | | GAAGCGATGCAAAATAGAATACAAATTGACCCAGTCAAATTGAGTA |
| | | GTGGCTACAAAGATGTGATACTTTGGTTTAGCTTCGGGGCATCATG |
| | | CTTTTTGCTTCTTGCCATTGCAATGGGCCTTGTTTTCATATGTGTG |
| | | AAGAACGGAAACATGCGGTGCACTATTTGTATATAA |
| 44 | Envelope; | AGTGTAACAGAGCACTTTAATGTGTATAAGGCTACTAGACCATACC |
| | RRV | TAGCACATTGCGCCGATTGCGGGGACGGGTACTTCTGCTATAGCCC |
| | | AGTTGCTATCGAGGAGATCCGAGATGAGGCGTCTGATGGCATGCTT |
| | | AAGATCCAAGTCTCCGCCCAAATAGGTCTGGACAAGGCAGGCACCC |
| | | ACGCCCACACGAAGCTCCGATATATGGCTGGTCATGATGTTCAGGA |
| | | ATCTAAGAGAGATTCCTTGAGGGTGTACACGTCCGCAGCGTGCTCC |
| | | ATACATGGGACGATGGGACACTTCATCGTCGCACACTGTCCACCAG |
| | | GCGACTACCTCAAGGTTTCGTTCGAGGACGCAGATTCGCACGTGAA |
| | | GGCATGTAAGGTCCAATACAAGCACAATCCATTGCCGGTGGGTAGA |
| | | GAGAAGTTCGTGGTTAGACCACACTTTGGCGTAGAGCTGCCATGCA |
| | | CCTCATACCAGCTGACAACGGCTCCCACCGACGAGGAGATTGACAT |
| | | GCATACACCGCCAGATATACCGGATCGCACCCTGCTATCACAGACG |
| | | GCGGGCAACGTCAAAATAACAGCAGGCGGCAGGACTATCAGGTACA |
| | | ACTGTACCTGCGGCCGTGACAACGTAGGCACTACCAGTACTGACAA |
| | | GACCATCAACACATGCAAGATTGACCAATGCCATGCTGCCGTCACC |
| | | AGCCATGACAAATGGCAATTTACCTCTCCATTTGTTCCCAGGGCTG |
| | | ATCAGACAGCTAGGAAAGGCAAGGTACACGTTCCGTTCCCTCTGAC |
| | | TAACGTCACCTGCCGAGTGCCGTTGGCTCGAGCGCCGGATGCCACC |
| | | TATGGTAAGAAGGAGGTGACCCTGAGATTACACCCAGATCATCCGA |
| | | CGCTCTTCTCCTATAGGAGTTTAGGAGCCGAACCGCACCCGTACGA |
| | | GGAATGGGTTGACAAGTTCTCTGAGCGCATCATCCCAGTGACGGAA |
| | | GAAGGGATTGAGTACCAGTGGGGCAACAACCCGCCGGTCTGCCTGT |
| | | GGGCGCAACTGACGACCGAGGGCAAACCCCATGGCTGGCCACATGA |
| | | AATCATTCAGTACTATTATGGACTATACCCCGCCGCCACTATTGCC |
| | | GCAGTATCCGGGGCGAGTCTGATGGCCCTCCTAACTCTGGCGGCCA |
| | | CATGCTGCATGCTGGCCACCGCGAGGAGAAAGTGCCTAACACCGTA |
| | | CGCCCTGACGCCAGGAGCGGTGGTACCGTTGACACTGGGGCTGCTT |
| | | TGCTGCGCACCGAGGGCGAATGCA |
| 45 | Envelope; | ATGGAAGGTCCAGCGTTCTCAAAACCCCTTAAAGATAAGATTAACC |
| | MLV 10A1 | CGTGGAAGTCCTTAATGGTCATGGGGGTCTATTTAAGAGTAGGGAT |
| | | GGCAGAGAGCCCCCATCAGGTCTTTAATGTAACCTGGAGAGTCACC |
| | | AACCTGATGACTGGGCGTACCGCCAATGCCACCTCCCTTTTAGGAA |
| | | CTGTACAAGATGCCTTCCCAAGATTATATTTTGATCTATGTGATCT |
| | | GGTCGGAGAAGAGTGGGACCCTTCAGACCAGGAACCATATGTCGGG |
| | | TATGGCTGCAAATACCCCGGAGGGAGAAAGCGGACCCGGACTTTTG |
| | | ACTTTTACGTGTGCCCTGGGCATACCGTAAAATCGGGGTGTGGGGG |
| | | GCCAAGAGAGGGCTACTGTGGTGAATGGGGTTGTGAAACCACCGGA |
| | | CAGGCTTACTGGAAGCCCACATCATCATGGGACCTAATCTCCCTTA |
| | | AGCGCGGTAACACCCCCTGGGACACGGGATGCTCCAAAATGGCTTG |
| | | TGGCCCCTGCTACGACCTCTCCAAAGTATCCAATTCCTTCCAAGGG |
| | | GCTACTCGAGGGGGCAGATGCAACCCTCTAGTCCTAGAATTCACTG |
| | | ATGCAGGAAAAAAGGCTAATTGGGACGGGCCCAAATCGTGGGGACT |
| | | GAGACTGTACCGGACAGGAACAGATCCTATTACCATGTTCTCCCTG |
| | | ACCCGCCAGGTCCTCAATATAGGGCCCCGCATCCCCATTGGGCCTA |
| | | ATCCCGTGATCACTGGTCAACTACCCCCCTCCCGACCCGTGCAGAT |
| | | CAGGCTCCCCAGGCCTCCTCAGCCTCCTCCTACAGGCGCAGCCTCT |
| | | ATAGTCCCTGAGACTGCCCCACCTTCTCAACAACCTGGGACGGGAG |
| | | ACAGGCTGCTAAACCTGGTAGAAGGAGCCTATCAGGCGCTTAACCT |
| | | CACCAATCCCGACAAGACCCAAGAATGTTGGCTGTGCTTAGTGTCG |
| | | GGACCTCCTTATTACGAAGGAGTAGCGGTCGTGGGCACTTATACCA |
| | | ATCATTCTACCGCCCCGGCCAGCTGTACGGCCACTTCCCAACATAA |
| | | GCTTACCCTATCTGAAGTGACAGGACAGGGCCTATGCATGGGAGCA |
| | | CTACCTAAAACTCACCAGGCCTTATGTAACACCACCCAAAGTGCCG |
| | | GCTCAGGATCCTACTACCTTGCAGCACCCGCTGGAACAATGTGGGC |
| | | TTGTAGCACTGGATTGACTCCCTGCTTGTCCACCACGATGCTCAAT |
| | | CTAACCACAGACTATTGTGTATTAGTTGAGCTCTGGCCCAGAATAA |
| | | TTTACCACTCCCCCGATTATATGTATGGTCAGCTTGAACAGCGTAC |
| | | CAAATATAAGAGGGAGCCAGTATCGTTGACCCTGGCCCTTCTGCTA |
| | | GGAGGATTAACCATGGGAGGGATTGCAGCTGGAATAGGGACGGGGA |
| | | CCACTGCCCTAATCAAAACCCAGCAGTTTGAGCAGCTTCACGCCGC |
| | | TATCCAGACAGACCTCAACGAAGTCGAAAAATCAATTACCAACCTA |
| | | GAAAAGTCACTGACCTCGTTGTCTGAAGTAGTCCTACAGAACCGAA |
| | | GAGGCCTAGATTTGCTCTTCCTAAAAGAGGGAGGTCTCTGCGCAGC |
| | | CCTAAAAGAAGAATGTTGTTTTTATGCAGACCACACGGGACTAGTG |
| | | AGAGACAGCATGGCCAAACTAAGGGAAAGGCTTAATCAGAGACAAA |
| | | AACTATTTGAGTCAGGCCAAGGTTGGTTCGAAGGGCAGTTTAATAG |
| | | ATCCCCCTGGTTTACCACCTTAATCTCCACCATCATGGGACCTCTA |
| | | ATAGTACTCTTACTGATCTTACTCTTTGGACCCTGCATTCTCAATC |
| | | GATTGGTCCAATTTGTTAAAGACAGGATCTCAGTGGTCCAGGCTCT |
| | | GGTTTTGACTCAACAATATCACCAGCTAAAACCTATAGAGTACGAG |
| | | CCATGA |
| 46 | Envelope; | ATGGGTGTTACAGGAATATTGCAGTTACCTCGTGATCGATTCAAGA |
| | Ebola | GGACATCATTCTTTCTTTGGGTAATTATCCTTTTCCAAAGAACATT |
| | | TTCCATCCCACTTGGAGTCATCCACAATAGCACATTACAGGTTAGT |
| | | GATGTCGACAAACTGGTTTGCCGTGACAAACTGTCATCCACAAATC |
| | | AATTGAGATCAGTTGGACTGAATCTCGAAGGGAATGGAGTGGCAAC |
| | | TGACGTGCCATCTGCAACTAAAAGATGGGGCTTCAGGTCCGGTGTC |
| | | CCACCAAAGGTGGTCAATTATGAAGCTGGTGAATGGGCTGAAAACT |
| | | GCTACAATCTTGAAATCAAAAAACCTGACGGGAGTGAGTGTCTACC |
| | | AGCAGCGCCAGACGGGATTCGGGGCTTCCCCCGGTGCCGGTATGTG |
| | | CACAAAGTATCAGGAACGGGACCGTGTGCCGGAGACTTTGCCTTCC |
| | | ACAAAGAGGGTGCTTTCTTCCTGTATGACCGACTTGCTTCCACAGT |
| | | TATCTACCGAGGAACGACTTTCGCTGAAGGTGTCGTTGCATTTCTG |
| | | ATACTGCCCCAAGCTAAGAAGGACTTCTTCAGCTCACACCCCTTGA |
| | | GAGAGCCGGTCAATGCAACGGAGGACCCGTCTAGTGGCTACTATTC |
| | | TACCACAATTAGATATCAAGCTACCGGTTTTGGAACCAATGAGACA |
| | | GAGTATTTGTTCGAGGTTGACAATTTGACCTACGTCCAACTTGAAT |
| | | CAAGATTCACACCACAGTTTCTGCTCCAGCTGAATGAGACAATATA |
| | | TACAAGTGGGAAAAGGAGCAATACCACGGGAAAACTAATTTGGAAG |
| | | GTCAACCCCGAAATTGATACAACAATCGGGGAGTGGGCCTTCTGGG |
| | | AAACTAAAAAAACCTCACTAGAAAAATTCGCAGTGAAGAGTTGTCT |
| | | TTCACAGCTGTATCAAACAGAGCCAAAAACATCAGTGGTCAGAGTC |
| | | CGGCGCGAACTTCTTCCGACCCAGGGACCAACACAACAACTGAAGA |
| | | CCACAAAATCATGGCTTCAGAAAATTCCTCTGCAATGGTTCAAGTG |
| | | CACAGTCAAGGAAGGGAAGCTGCAGTGTCGCATCTGACAACCCTTG |
| | | CCACAATCTCCACGAGTCCTCAACCCCCCACAACCAAACCAGGTCC |
| | | GGACAACAGCACCCACAATACACCCGTGTATAAACTTGACATCTCT |
| | | GAGGCAACTCAAGTTGAACAACATCACCGCAGAACAGACAACGACA |
| | | GCACAGCCTCCGACACTCCCCCCGCCACGACCGCAGCCGGACCCCT |
| | | AAAAGCAGAGAACACCAACACGAGCAAGGGTACCGACCTCCTGGAC |
| | | CCCGCCACCACAACAAGTCCCCAAAACCACAGCGAGACCGCTGGCA |
| | | ACAACAACACTCATCACCAAGATACCGGAGAAGAGAGTGCCAGCAG |
| | | CGGGAAGCTAGGCTTAATTACCAATACTATTGCTGGAGTCGCAGGA |
| | | CTGATCACAGGCGGGAGGAGAGCTCGAAGAGAAGCAATTGTCAATG |
| | | CTCAACCCAAATGCAACCCTAATTTACATTACTGGACTACTCAGGA |
| | | TGAAGGTGCTGCAATCGGACTGGCCTGGATACCATATTTCGGGCCA |
| | | GCAGCCGAGGGAATTTACATAGAGGGGCTGATGCACAATCAAGATG |
| | | GTTTAATCTGTGGGTTGAGACAGCTGGCCAACGAGACGACTCAAGC |
| | | TCTTCAACTGTTCCTGAGAGCCACAACCGAGCTACGCACCTTTTCA |
| | | ATCCTCAACCGTAAGGCAATTGATTTCTTGCTGCAGCGATGGGGCG |
| | | GCACATGCCACATTTTGGGACCGGACTGCTGTATCGAACCACATGA |
| | | TTGGACCAAGAACATAACAGACAAAATTGATCAGATTATTCATGAT |
| | | TTTGTTGATAAAACCCTTCCGGACCAGGGGGACAATGACAATTGGT |
| | | GGACAGGATGGAGACAATGGATACCGGCAGGTATTGGAGTTACAGG |
| | | CGTTATAATTGCAGTTATCGCTTTATTCTGTATATGCAAATTTGTC |
| | | TTTTAG |
| 47 | Left ITR | CCTGCAGGCAGCTGCGCGCTCGCTCGCTCACTGAGGCCGCCCGGGC |
| | | AAAGCCCGGGCGTCGGGCGACCTTTGGTCGCCCGGCCTCAGTGAGC |
| | | GAGCGAGCGCGCAGAGAGGGAGTGGCCAACTCCATCACTAGGGGTT |
| | | CCT |
| 48 | Prothrombin | GCGAGAACTTGTGCCTCCCCGTGTTCCTGCTCTTTGTCCCTCTGTC |
| | enhancer | CTACTTAGACTAATATTTGCCTTGGGTACTGCAAACAGGAAATGGG |
| | | GGAGGGACAGGAGTAGGGCGGAGGGTAG |
| 49 | PolyA | GACTGTGCCTTCTAGTTGCCAGCCATCTGTTGTTTGCCCCTCCCCC |
| | | GTGCCTTCCTTGACCCTGGAAGGTGCCACTCCCACTGTCCTTTCCT |
| | | AATAAAATGAGGAAATTGCATCGCATTGTCTGAGTAGGTGTCATTC |
| | | TATTCTGGGGGGTGGGGTGGGGCAGGACAGCAAGGGGGAGGATTGG |
| | | GAAGACAATAGCAGGCATGCTGGGGATGCGGTGGGCTCTATGGC |
| 50 | Right ITR | AGGAACCCCTAGTGATGGAGTTGGCCACTCCCTCTCTGCGCGCTCG |
| | | CTCGCTCACTGAGGCCGGGCGACCAAAGGTCGCCCGACGCCCGGGC |
| | | TTTGCCCGGGCGGCCTCAGTGAGCGAGCGAGCGCGCAGCTGCCTGC |
| | | AGG |
| 51 | E2A | TTAAAAGTCGAAGGGGTTCTCGCGCTCGTCGTTGTGCGCCGCGCTG |
| | | GGGAGGGCCACGTTGCGGAACTGGTACTTGGGCTGCCACTTGAACT |
| | | CGGGGATCACCAGTTTGGGCACTGGGGTCTCGGGGAAGGTCTCGCT |
| | | CCACATGCGCCGGCTCATCTGCAGGGCGCCCAGCATGTCAGGCGCG |
| | | GAGATCTTGAAATCGCAGTTGGGGCCGGTGCTCTGCGCGCGCGAGT |
| | | TGCGGTACACTGGGTTGCAGCACTGGAACACCATCAGACTGGGGTA |
| | | CTTCACACTAGCCAGCACGCTCTTGTCGCTGATCTGATCCTTGTCC |
| | | AGGTCCTCGGCGTTGCTCAGGCCGAACGGGGTCATCTTGCACAGCT |
| | | GGCGGCCCAGGAAGGGCACGCTCTGAGGCTTGTGGTTACACTCGCA |
| | | GTGCACGGGCATCAGCATCATCCCCGCGCCGCGCTGCATATTCGGG |
| | | TAGAGGGCCTTGACGAAGGCCGCGATCTGCTTGAAAGCTTGCTGGG |
| | | CCTTGGCCCCCTCGCTGAAAAACAGGCCGCAGCTCTTCCCGCTGAA |
| | | CTGATTATTCCCGCACCCGGCATCATGGACGCAGCAGCGCGCGTCA |
| | | TGGCTGGTCAGTTGCACCACGCTCCGTCCCCAGCGGTTCTGGGTCA |
| | | CCTTGGCCTTGCTGGGTTGCTCCTTCAGCGCACGCTGCCCGTTCTC |
| | | ACTGGTCACATCCATCTCCACCACGTGGTCCTTGTGGATCATCACC |
| | | GTCCCATGCAGACACTTGAGCTGGCCTTCCACCTCGGTGCAGCCGT |
| | | GGTCCCACAGGGCACTGCCGGTGCACTCCCAGTTCTTGTGCGCGAT |
| | | CCCGCTGTGGCTGAAGATGTAACCTTGCAACAGGCGACCCATGATG |
| | | GTGCTAAAGCTCTTCTGGGTGGTGAAGGTCAGTTGCAGACCGCGGG |
| | | CCTCCTCGTTCATCCAGGTCTGGCACATCTTTTGGAAGATCTCGGT |
| | | CTGCTCGGGCATGAGCTTGTAAGCATCGCGCAGGCCGCTGTCGACG |
| | | CGGTAGCGTTCCATCAGCACATTCATGGTATCCATGCCCTTCTCCC |
| | | AGGACGAGACCAGAGGCAGACTCAGGGGGTTGCGCACGTTCAGGAC |
| | | ACCGGGGGTCGCGGGCTCGACGATGCGTTTTCCGTCCTTGCCTTCC |
| | | TTCAACAGAACCGGCGGCTGGCTGAATCCCACTCCCACGATCACGG |
| | | CTTCTTCCTGGGGCATCTCTTCGTCTGGGTCTACCTTGGTCACATG |
| | | CTTGGTCTTTCTGGCTTGCTTCTTTTTTGGAGGGCTGTCCACGGGG |
| | | ACCACGTCCTCCTCGGAAGACCCGGATCCCACCCGCTGATACTTTC |
| | | GGCGCTTGGTTGGCAGAGGAGGTGGCGGCGAGGGGCTCCTCTCCTG |
| | | CTCCGGCGGATAGCGCGCTGAACCGTGGCCCCGGGGCGGAGTGGCC |
| | | TCTCGGTCCATGAACCGGCGCACGTCCTGACTGCCGCCGGCCAT |
| 52 | E4 | TCATGTATCTTTATTGATTTTTACACCAGCACGGGTAGTCAGTCTC |
| | | CCACCACCAGCCCATTTCACAGTGTAAACAATTCTCTCAGCACGGG |
| | | TGGCCTTAAATAGGGCAATATTCTGATTAGTGCGGGAACTGGACTT |
| | | GGGGTCTATAATCCACACAGTTTCCTGGCGAGCCAAACGGGGGTCG |
| | | GTGATTGAGATGAAGCCGTCCTCTGAAAAGTCATCCAAGCGAGCCT |
| | | CACAGTCCAAGGTCACAGTATTATGATAATCTGCATGATCACAATC |
| | | GGGCAACAGGGGATGTTGTTCAGTCAGTGAAGCCCTGGTTTCCTCA |
| | | TCAGATCGTGGTAAACGGGCCCTGCGATATGGATGATGGCGGAGCG |
| | | AGCTGGATTGAATCTCGGTTTGCAT |
| 53 | VA RNA | AGCGGGCACTCTTCCGTGGTCTGGTGGATAAATTCGCAAGGGTATC |
| | | ATGGCGGACGACCGGGGTTCGAGCCCCGTATCCGGCCGTCCGCCGT |
| | | GATCCATGCGGTTACCGCCCGCGTGTCGAACCCAGGTGTGCGACGT |
| | | CAGACAACGGGGGAGTGCTCCTTT |
| 54 | AAV2 Rep | ATGGCTGCCGATGGTTATCTTCCAGATTGGCTCGAGGACACTCTCT |
| | | CTGAAGGAATAAGACAGTGGTGGAAGCTCAAACCTGGCCCACCACC |
| | | ACCAAAGCCCGCAGAGCGGCATAAGGACGACAGCAGGGGTCTTGTG |
| | | CTTCCTGGGTACAAGTACCTCGGACCCTTCAACGGACTCGACAAGG |
| | | GAGAGCCGGTCAACGAGGCAGACGCCGCGGCCCTCGAGCACGACAA |
| | | AGCCTACGACCGGCAGCTCGACAGCGGAGACAACCCGTACCTCAAG |
| | | TACAACCACGCCGACGCGGAGTTTCAGGAGCGCCTTAAAGAAGATA |
| | | CGTCTTTTGGGGGCAACCTCGGACGAGCAGTCTTCCAGGCGAAAAA |
| | | GAGGGTTCTTGAACCTCTGGGCCTGGTTGAGGAACCTGTTAAGACG |
| | | GCTCCGGGAAAAAAGAGGCCGGTAGAGCACTCTCCTGTGGAGCCAG |
| | | ACTCCTCCTCGGGAACCGGAAAGGCGGGCCAGCAGCCTGCAAGAAA |
| | | AAGATTGAATTTTGGTCAGACTGGAGACGCAGACTCAGTACCTGAC |
| | | CCCCAGCCTCTCGGACAGCCACCAGCAGCCCCCTCTGGTCTGGGAA |
| | | CTAATACGATGGCTACAGGCAGTGGCGCACCAATGGCAGACAATAA |
| | | CGAGGGCGCCGACGGAGTGGGTAATTCCTCGGGAAATTGGCATTGC |
| | | GATTCCACATGGATGGGCGACAGAGTCATCACCACCAGCACCCGAA |
| | | CCTGGGCCCTGCCCACCTACAACAACCACCTCTACAAACAAATTTC |
| | | CAGCCAATCAGGAGCCTCGAACGACAATCACTACTTTGGCTACAGC |
| | | ACCCCTTGGGGGTATTTTGACTTCAACAGATTCCACTGCCACTTTT |
| | | CACCACGTGACTGGCAAAGACTCATCAACAACAACTGGGGATTCCG |
| | | ACCCAAGAGACTCAACTTCAAGCTCTTTAACATTCAAGTCAAAGAG |
| | | GTCACGCAGAATGACGGTACGACGACGATTGCCAATAACCTTACCA |
| | | GCACGGTTCAGGTGTTTACTGACTCGGAGTACCAGCTCCCGTACGT |
| | | CCTCGGCTCGGCGCATCAAGGATGCCTCCCGCCGTTCCCAGCAGAC |
| | | GTCTTCATGGTGCCACAGTATGGATACCTCACCCTGAACAACGGGA |
| | | GTCAGGCAGTAGGACGCTCTTCATTTTACTGCCTGGAGTACTTTCC |
| | | TTCTCAGATGCTGCGTACCGGAAACAACTTTACCTTCAGCTACACT |
| | | TTTGAGGACGTTCCTTTCCACAGCAGCTACGCTCACAGCCAGAGTC |
| | | TGGACCGTCTCATGAATCCTCTCATCGACCAGTACCTGTATTACTT |
| | | GAGCAGAACAAACACTCCAAGTGGAACCACCACGCAGTCAAGGCTT |
| | | CAGTTTTCTCAGGCCGGAGCGAGTGACATTCGGGACCAGTCTAGGA |
| | | ACTGGCTTCCTGGACCCTGTTACCGCCAGCAGCGAGTATCAAAGAC |
| | | ATCTGCGGATAACAACAACAGTGAATACTCGTGGACTGGAGCTACC |
| | | AAGTACCACCTCAATGGCAGAGACTCTCTGGTGAATCCGGGCCCGG |
| | | CCATGGCAAGCCACAAGGACGATGAAGAAAAGTTTTTTCCTCAGAG |
| | | CGGGGTTCTCATCTTTGGGAAGCAAGGCTCAGAGAAAACAAATGTG |
| | | GACATTGAAAAGGTCATGATTACAGACGAAGAGGAAATCAGGACAA |
| | | CCAATCCCGTGGCTACGGAGCAGTATGGTTCTGTATCTACCAACCT |
| | | CCAGAGAGGCAACAGACAAGCAGCTACCGCAGATGTCAACACACAA |
| | | GGCGTTCTTCCAGGCATGGTCTGGCAGGACAGAGATGTGTACCTTC |
| | | AGGGGCCCATCTGGGCAAAGATTCCACACACGGACGGACATTTTCA |
| | | CCCCTCTCCCCTCATGGGTGGATTCGGACTTAAACACCCTCCTCCA |
| | | CAGATTCTCATCAAGAACACCCCGGTACCTGCGAATCCTTCGACCA |
| | | CCTTCAGTGCGGCAAAGTTTGCTTCCTTCATCACACAGTACTCCAC |
| | | GGGACAGGTCAGCGTGGAGATCGAGTGGGAGCTGCAGAAGGAAAAC |
| | | AGCAAACGCTGGAATCCCGAAATTCAGTACACTTCCAACTACAACA |
| | | AGTCTGTTAATGTGGACTTTACTGTGGACACTAATGGCGTGTATTC |
| | | AGAGCCTCGCCCCATTGGCACCAGATACCTGACTCGTAATCTGTAA |
| 55 | AAV2 Cap | ATGCCGGGGTTTTACGAGATTGTGATTAAGGTCCCCAGCGACCTTG |
| | | ACGAGCATCTGCCCGGCATTTCTGACAGCTTTGTGAACTGGGTGGC |
| | | CGAGAAGGAATGGGAGTTGCCGCCAGATTCTGACATGGATCTGAAT |
| | | CTGATTGAGCAGGCACCCCTGACCGTGGCCGAGAAGCTGCAGCGCG |
| | | ACTTTCTGACGGAATGGCGCCGTGTGAGTAAGGCCCCGGAGGCCCT |
| | | TTTCTTTGTGCAATTTGAGAAGGGAGAGAGCTACTTCCACATGCAC |
| | | GTGCTCGTGGAAACCACCGGGGTGAAATCCATGGTTTTGGGACGTT |
| | | TCCTGAGTCAGATTCGCGAAAAACTGATTCAGAGAATTTACCGCGG |
| | | GATCGAGCCGACTTTGCCAAACTGGTTCGCGGTCACAAAGACCAGA |
| | | AATGGCGCCGGAGGCGGGAACAAGGTGGTGGATGAGTGCTACATCC |
| | | CCAATTACTTGCTCCCCAAAACCCAGCCTGAGCTCCAGTGGGCGTG |
| | | GACTAATATGGAACAGTATTTAAGCGCCTGTTTGAATCTCACGGAG |
| | | CGTAAACGGTTGGTGGCGCAGCATCTGACGCACGTGTCGCAGACGC |
| | | AGGAGCAGAACAAAGAGAATCAGAATCCCAATTCTGATGCGCCGGT |
| | | GATCAGATCAAAAACTTCAGCCAGGTACATGGAGCTGGTCGGGTGG |
| | | CTCGTGGACAAGGGGATTACCTCGGAGAAGCAGTGGATCCAGGAGG |
| | | ACCAGGCCTCATACATCTCCTTCAATGCGGCCTCCAACTCGCGGTC |
| | | CCAAATCAAGGCTGCCTTGGACAATGCGGGAAAGATTATGAGCCTG |
| | | ACTAAAACCGCCCCCGACTACCTGGTGGGCCAGCAGCCCGTGGAGG |
| | | ACATTTCCAGCAATCGGATTTATAAAATTTTGGAACTAAACGGGTA |
| | | CGATCCCCAATATGCGGCTTCCGTCTTTCTGGGATGGGCCACGAAA |
| | | AAGTTCGGCAAGAGGAACACCATCTGGCTGTTTGGGCCTGCAACTA |
| | | CCGGGAAGACCAACATCGCGGAGGCCATAGCCCACACTGTGCCCTT |
| | | CTACGGGTGCGTAAACTGGACCAATGAGAACTTTCCCTTCAACGAC |
| | | TGTGTCGACAAGATGGTGATCTGGTGGGAGGAGGGGAAGATGACCG |
| | | CCAAGGTCGTGGAGTCGGCCAAAGCCATTCTCGGAGGAAGCAAGGT |
| | | GCGCGTGGACCAGAAATGCAAGTCCTCGGCCCAGATAGACCCGACT |
| | | CCCGTGATCGTCACCTCCAACACCAACATGTGCGCCGTGATTGACG |
| | | GGAACTCAACGACCTTCGAACACCAGCAGCCGTTGCAAGACCGGAT |
| | | GTTCAAATTTGAACTCACCCGCCGTCTGGATCATGACTTTGGGAAG |
| | | GTCACCAAGCAGGAAGTCAAAGACTTTTTCCGGTGGGCAAAGGATC |
| | | ACGTGGTTGAGGTGGAGCATGAATTCTACGTCAAAAAGGGTGGAGC |
| | | CAAGAAAAGACCCGCCCCCAGTGACGCAGATATAAGTGAGCCCAAA |
| | | CGGGTGCGCGAGTCAGTTGCGCAGCCATCGACGTCAGACGCGGAAG |
| | | CTTCGATCAACTACGCAGACAGGTACCAAAACAAATGTTCTCGTCA |
| | | CGTGGGCATGAATCTGATGCTGTTTCCCTGCAGACAATGCGAGAGA |
| | | ATGAATCAGAATTCAAATATCTGCTTCACTCACGGACAGAAAGACT |
| | | GTTTAGAGTGCTTTCCCGTGTCAGAATCTCAACCCGTTTCTGTCGT |
| | | CAAAAAGGCGTATCAGAAACTGTGCTACATTCATCATATCATGGGA |
| | | AAGGTGCCAGACGCTTGCACTGCCTGCGATCTGGTCAATGTGGATT |
| | | TGGATGACTGCATCTTTGAACAATAA |
| 56 | DNA | TCTAGAAGGAGCTTTGTTCCTTGGGTTCTTGGGAGCAGCAGGAAGC |
| | Fragment | ACTATGGGCGCAGCGTCAATGACGCTGACGGTACAGGCCAGACAAT |
| | containing | TATTGTCTGGTATAGTGCAGCAGCAGAACAATTTGCTGAGGGCTAT |
| | RRE and | TGAGGCGCAACAGCATCTGTTGCAACTCACAGTCTGGGGCATCAAG |
| | rabbit beta | CAGCTCCAGGCAAGAATCCTGGCTGTGGAAAGATACCTAAAGGATC |
| | globin poly A | AACAGCTCCTAGATCTTTTTCCCTCTGCCAAAAATTATGGGGACAT |
| | | CATGAAGCCCCTTGAGCATCTGACTTCTGGCTAATAAAGGAAATTT |
| | | ATTTTCATTGCAATAGTGTGTTGGAATTTTTTGTGTCTCTCACTCG |
| | | GAAGGACATATGGGAGGGCAAATCATTTAAAACATCAGAATGAGTA |
| | | TTTGGTTTAGAGTTTGGCAACATATGCCATATGCTGGCTGCCATGA |
| | | ACAAAGGTGGCTATAAAGAGGTCATCAGTATATGAAACAGCCCCCT |
| | | GCTGTCCATTCCTTATTCCATAGAAAAGCCTTGACTTGAGGTTAGA |
| | | TTTTTTTTATATTTTGTTTTGTGTTATTTTTTTCTTTAACATCCCT |
| | | AAAATTTTCCTTACATGTTTTACTAGCCAGATTTTTCCTCCTCTCC |
| | | TGACTACTCCCAGTCATAGCTGTCCCTCTTCTCTTATGAAGATCCC |
| | | TCGACCTGCAGCCCAAGCTTGGCGTAATCATGGTCATAGCTGTTTC |
| | | CTGTGTGAAATTGTTATCCGCTCACAATTCCACACAACATACGAGC |
| | | CGGAAGCATAAAGTGTAAAGCCTGGGGTGCCTAATGAGTGAGCTAA |
| | | CTCACATTAATTGCGTTGCGCTCACTGCCCGCTTTCCAGTCGGGAA |
| | | ACCTGTCGTGCCAGCGGATCCGCATCTCAATTAGTCAGCAACCATA |
| | | GTCCCGCCCCTAACTCCGCCCATCCCGCCCCTAACTCCGCCCAGTT |
| | | CCGCCCATTCTCCGCCCCATGGCTGACTAATTTTTTTTATTTATGC |
| | | AGAGGCCGAGGCCGCCTCGGCCTCTGAGCTATTCCAGAAGTAGTGA |
| | | GGAGGCTTTTTTGGAGGCCTAGGCTTTTGCAAAAAGCTAACTTGTT |
| | | TATTGCAGCTTATAATGGTTACAAATAAAGCAATAGCATCACAAAT |
| | | TTCACAAATAAAGCATTTTTTTCACTGCATTCTAGTTGTGGTTTGT |
| | | CCAAACTCATCAATGTATCTTATCACCCGGG |
| 57 | hPAH with fill | GGCACGAGGTACCTGAGGCCCTAAAAAGCCAGAGACCTCACTCCCG |
| | 5′ and 3′ UTR | GGGAGCCAGCATGTCCACTGCGGTCCTGGAAAACCCAGGCTTGGGC |
| | | AGGAAACTCTCTGACTTTGGACAGGAAACAAGCTATATTGAAGACA |
| | | ACTGCAATCAAAATGGTGCCATATCACTGATCTTCTCACTCAAAGA |
| | | AGAAGTTGGTGCATTGGCCAAAGTATTGCGCTTATTTGAGGAGAAT |
| | | GATGTAAACCTGACCCACATTGAATCTAGACCTTCTCGTTTAAAGA |
| | | AAGATGAGTATGAATTTTTCACCCATTTGGATAAACGTAGCCTGCC |
| | | TGCTCTGACAAACATCATCAAGATCTTGAGGCATGACATTGGTGCC |
| | | ACTGTCCATGAGCTTTCACGAGATAAGAAGAAAGACACAGTGCCCT |
| | | GGTTCCCAAGAACCATTCAAGAGCTGGACAGATTTGCCAATCAGAT |
| | | TCTCAGCTATGGAGCGGAACTGGATGCTGACCACCCTGGTTTTAAA |
| | | GATCCTGTGTACCGTGCAAGACGGAAGCAGTTTGCTGACATTGCCT |
| | | ACAACTACCGCCATGGGCAGCCCATCCCTCGAGTGGAATACATGGA |
| | | GGAAGAAAAGAAAACATGGGGCACAGTGTTCAAGACTCTGAAGTCC |
| | | TTGTATAAAACCCATGCTTGCTATGAGTACAATCACATTTTTCCAC |
| | | TTCTTGAAAAGTACTGTGGCTTCCATGAAGATAACATTCCCCAGCT |
| | | GGAAGACGTTTCTCAGTTCCTGCAGACTTGCACTGGTTTCCGCCTC |
| | | CGACCTGTAGCTGGCCTGCTTTCCTCTCGGGATTTCTTGGGTGGCC |
| | | TGGCCTTCCGAGTCTTCCACTGCACACAGTACATCAGACATGGATC |
| | | CAAGCCCATGTATACCCCCGAACCTGACATCTGCCATGAGCTGTTG |
| | | GGACATGTGCCCTTGTTTTCAGATCGCAGCTTTGCCCAGTTTTCCC |
| | | AGGAAATTGGCCTTGCCTCTCTGGGTGCACCTGATGAATACATTGA |
| | | AAAGCTCGCCACAATTTACTGGTTTACTGTGGAGTTTGGGCTCTGC |
| | | AAACAAGGAGACTCCATAAAGGCATATGGTGCTGGGCTCCTGTCAT |
| | | CCTTTGGTGAATTACAGTACTGCTTATCAGAGAAGCCAAAGCTTCT |
| | | CCCCCTGGAGCTGGAGAAGACAGCCATCCAAAATTACACTGTCACG |
| | | GAGTTCCAGCCCCTGTATTACGTGGCAGAGAGTTTTAATGATGCCA |
| | | AGGAGAAAGTAAGGAACTTTGCTGCCACAATACCTCGGCCCTTCTC |
| | | AGTTCGCTACGACCCATACACCCAAAGGATTGAGGTCTTGGACAAT |
| | | ACCCAGCAGCTTAAGATTTTGGCTGATTCCATTAACAGTGAAATTG |
| | | GAATCCTTTGCAGTGCCCTCCAGAAAATAAAGTAAAGCCATGGACA |
| | | GAATGTGGTCTGTCAGCTGTGAATCTGTTGATGGAGATCCAACTAT |
| | | TTCTTTCATCAGAAAAAGTCCGAAAAGCAAACCTTAATTTGAAATA |
| | | ACAGCCTTAAATCCTTTACAAGATGGAGAAACAACAAATAAGTCAA |
| | | AATAATCTGAAATGACAGGATATGAGTACATACTCAAGAGCATAAT |
| | | GGTAAATCTTTTGGGGTCATCTTTGATTTAGAGATGATAATCCCAT |
| | | ACTCTCAATTGAGTTAAATCAGTAATCTGTCGCATTTCATCAAGAT |
| | | TAATTAAAATTTGGGACCTGCTTCATTCAAGCTTCATATATGCTTT |
| | | GCAGAGAACTCATAAAGGAGCATATAAGGCTAAATGTAAAACCCAA |
| | | GACTGTCATTAGAATTGAATTATTGGGCTTAATATAAATCGTAACC |
| | | TATGAAGTTTATTTTTTATTTTAGTTAACTATGATTCCAATTACTA |
| | | CTTTGTTATTGTACCTAAGTAAATTTTCTTTAAGTCAGAAGCCCAT |
| | | TAAAATAGTTACAAGCATTGAACTTCTTTAGTATTATATTAATATA |
| | | AAAACATTTTTGTATGTTTTATTGTAATCATAAATACTGCTGTATA |
| | | AGGTAATAAAACTCTGCACCTAATCCCCATAACTTCCAGTATCATT |
| | | TTCCAATTAATTATCAAGTCTGTTTTGGGAAACACTTTGAGGACAT |
| | | TTATGATGCAGCAGATGTTGACTAAAGGCTTGGTTGGTAGATATTC |
| | | AGGAAATGTTCACTGAATAAATAAGTAAATACATTATTGAAAAGCA |
| | | AATCTGTATAAATGTGAAATTTTTATTTGTATTAGTAATAAAACAT |
| | | TAGTAGTTTAAACAAAAAAAAAAAAAAAAAAAAAAAAAAAAAAACT |
| | | CGACTCTAGATT |
| 58 | hPAH with | GGCACGAGGTACCTGAGGCCCTAAAAAGCCAGAGACCTCACTCCCG |
| | full 5′ UTR | GGGAGCCAGCATGTCCACTGCGGTCCTGGAAAACCCAGGCTTGGGC |
| | and truncated | AGGAAACTCTCTGACTTTGGACAGGAAACAAGCTATATTGAAGACA |
| | 3′ UTR | ACTGCAATCAAAATGGTGCCATATCACTGATCTTCTCACTCAAAGA |
| | | AGAAGTTGGTGCATTGGCCAAAGTATTGCGCTTATTTGAGGAGAAT |
| | | GATGTAAACCTGACCCACATTGAATCTAGACCTTCTCGTTTAAAGA |
| | | AAGATGAGTATGAATTTTTCACCCATTTGGATAAACGTAGCCTGCC |
| | | TGCTCTGACAAACATCATCAAGATCTTGAGGCATGACATTGGTGCC |
| | | ACTGTCCATGAGCTTTCACGAGATAAGAAGAAAGACACAGTGCCCT |
| | | GGTTCCCAAGAACCATTCAAGAGCTGGACAGATTTGCCAATCAGAT |
| | | TCTCAGCTATGGAGCGGAACTGGATGCTGACCACCCTGGTTTTAAA |
| | | GATCCTGTGTACCGTGCAAGACGGAAGCAGTTTGCTGACATTGCCT |
| | | ACAACTACCGCCATGGGCAGCCCATCCCTCGAGTGGAATACATGGA |
| | | GGAAGAAAAGAAAACATGGGGCACAGTGTTCAAGACTCTGAAGTCC |
| | | TTGTATAAAACCCATGCTTGCTATGAGTACAATCACATTTTTCCAC |
| | | TTCTTGAAAAGTACTGTGGCTTCCATGAAGATAACATTCCCCAGCT |
| | | GGAAGACGTTTCTCAGTTCCTGCAGACTTGCACTGGTTTCCGCCTC |
| | | CGACCTGTAGCTGGCCTGCTTTCCTCTCGGGATTTCTTGGGTGGCC |
| | | TGGCCTTCCGAGTCTTCCACTGCACACAGTACATCAGACATGGATC |
| | | CAAGCCCATGTATACCCCCGAACCTGACATCTGCCATGAGCTGTTG |
| | | GGACATGTGCCCTTGTTTTCAGATCGCAGCTTTGCCCAGTTTTCCC |
| | | AGGAAATTGGCCTTGCCTCTCTGGGTGCACCTGATGAATACATTGA |
| | | AAAGCTCGCCACAATTTACTGGTTTACTGTGGAGTTTGGGCTCTGC |
| | | AAACAAGGAGACTCCATAAAGGCATATGGTGCTGGGCTCCTGTCAT |
| | | CCTTTGGTGAATTACAGTACTGCTTATCAGAGAAGCCAAAGCTTCT |
| | | CCCCCTGGAGCTGGAGAAGACAGCCATCCAAAATTACACTGTCACG |
| | | GAGTTCCAGCCCCTGTATTACGTGGCAGAGAGTTTTAATGATGCCA |
| | | AGGAGAAAGTAAGGAACTTTGCTGCCACAATACCTCGGCCCTTCTC |
| | | AGTTCGCTACGACCCATACACCCAAAGGATTGAGGTCTTGGACAAT |
| | | ACCCAGCAGCTTAAGATTTTGGCTGATTCCATTAACAGTGAAATTG |
| | | GAATCCTTTGCAGTGCCCTCCAGAAAATAAAGTAAAGCCATGGACA |
| | | GAATGTGGTCTGTCAGCTGTGAATCTGTTGATGGAGATCCAACTAT |
| | | TTCTTTCATCAGAAAAAGTCCGAAAAGCAAACCTTAATTTGAAATA |
| | | ACAGCCTTAAATCCTTTACAAGATGGAGAAACAACAAATAAGTCAA |
| | | AATAATCTGAAATGACAGGATATGAGTACATACTCAAGAGCATAAT |
| | | GGTAAATCTTTTGGGGTCATCTTTGATTTAGAGATGATAATCCCAT |
| | | ACTCTCAATTGAGTTAAATCAGTAATCTGTCGCATTTCATCAAGAT |
| | | TA |

What is claimed is:

1. A viral vector comprising:
a phenylalanine hydroxylase (PAH) sequence for expressing at least one of PAH enzyme or a variant thereof; and
at least one small RNA sequence that is capable of binding to at least one pre-determined complementary mRNA sequence,
wherein the at least one small RNA sequence does not target the at least one of PAH sequence.

2. The viral vector of claim 1, wherein the at least one pre-determined complementary mRNA sequence is a PAH mRNA sequence.

3. The viral vector of claim 1, wherein the at least one small RNA sequence is under the control of a first promoter, and wherein the PAH sequence is under the control of a second promoter.

4. The viral vector of claim 3, wherein the first promoter comprises an H1 promoter and wherein the second promoter comprises a liver-specific promoter.

5. The viral vector of claim 4, wherein the liver-specific promoter comprises a hAAT promoter.

6. The viral vector of claim 1, wherein the PAH sequence comprises at least one of 80%, 85%, 90%, 95%, or 100% identity with at least one of SEQ ID NO: 1, SEQ ID NO: 2, SEQ ID NO: 3, or SEQ ID NO: 4.

7. The viral vector of claim 1, wherein the PAH sequence is truncated at a 3′ untranslated region (UTR) of the sequence.

8. The viral vector of claim 7, wherein the truncated sequence comprises a sequence having at least one of 80%, 85%, 90%, 95%, or 100% identity with at least one of SEQ ID NO: 3 or SEQ ID NO: 4.

9. The viral vector of claim 1, wherein the at least one small RNA sequence comprises a sequence having at least one of 80%, 85%, 90%, 95%, or 100% identity with at least one of SEQ ID NO: 5 or SEQ ID NO: 6.

10. A lentiviral particle produced by a packaging cell and capable of infecting a target cell, the lentiviral particle comprising:
an envelope protein capable of infecting a target cell; and
the viral vector according to claim 1.

11. The lentiviral particle of claim 10, wherein the target cell is at least one of a hepatic cell, a muscle cell, an epithelial cell, an endothelial cell, a neural cell, a neuroendocrine cell, an endocrine cell, a lymphocyte, a myeloid cell, a cell present within a solid organ, or cell of a hematopoietic lineage, a hematopoietic stem cell, or a precursor hematopoietic stem cell.


SEQUENCE LISTING (58 sequences)

SEQ ID NO: 1 - DNA, 1359 bp, synthetic construct
  misc_feature 1..1359: note = PAH
  source 1..1359: mol_type = other DNA
atgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcaattcctgcagacttgcactggtttccgcctccgacctgtggctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaa

SEQ ID NO: 2 - DNA, 1359 bp, synthetic construct
  misc_feature 1..1359: note = Codon optimized PAH
  source 1..1359: mol_type = other DNA
atgagcacagctgtgttggaaaatcctgggctgggccgtaagctttccgatttcggccaggagacttcatacattgaggacaactgcaaccagaatggggccatttctttgatcttcagtctcaaagaagaggtaggcgctctggctaaggtcctgaggctgtttgaggaaaatgacgtgaatctgacacacattgagtctaggccttcccgacttaagaaggatgagtatgagttcttcacacacctggacaaacgatctctcccagcactgaccaatatcatcaagattctcaggcatgatatcggtgccacggtccacgaactttcacgcgataagaagaaagacacagttccctggttcccgagaaccattcaggaactggataggtttgccaatcagattctgagctatggggcagagttggatgccgaccatccaggcttcaaagaccccgtatatcgggctcggagaaagcagtttgcagacatcgcttacaattacaggcatggacagcccatccctagagtggagtacatggaagaaggcaagaaaacctggggaacggtgtttaagaccctcaaaagcctgtataagacccacgcgtgttatgagtacaaccacattttcccattgctggagaagtactgtggctttcacgaggacaacatccctcaactggaggatgtttcacagttccttcagacttgcactggtttccgccttcgacctgtggctgggctgcttagctcacgggacttcctgggaggcctggccttcagagtctttcactgcactcagtacattcggcatggctctaagccaatgtacacccctgaaccggatatatgccacgagctgttgggacatgtgcccctgttttctgatcgcagctttgcccagttttcccaggagattggcctggcaagtcttggtgcgcctgatgagtacatcgagaagctcgcgacaatctactggttcaccgtggaatttggactctgcaaacaaggggactctatcaaagcctacggagcaggactcctctccagcttcggtgaactgcagtattgtctgtccgagaaacccaaactcttgcccctggaactggaaaagactgccatccaaaactatactgtcacggaatttcagccactgtattatgtggctgaatcctttaacgatgccaaggagaaggtccgtaattttgctgccacaataccacgccccttcagcgtgagatacgacccgtatacacaacggatagaggttctggacaacacccagcaactgaaaattctggcagacagtataaacagcgaaatagggatcctctgtagtgccctgcagaaaatcaaatga

SEQ ID NO: 3 - DNA, 897 bp, synthetic construct
  misc_feature 1..897: note = PAH 3'UTR sequence (897 nucleotides)
  source 1..897: mol_type = other DNA
agccatggacagaatgtggtctgtcagctgtgaatctgttgatggagatccaactatttctttcatcagaaaaagtccgaaaagcaaaccttaatttgaaataacagccttaaatcctttacaagatggagaaacaacaaataagtcaaaataatctgaaatgacaggatatgagtacatactcaagagcataatggtaaatcttttggggtcatctttgatttagagatgataatcccatactctcaattgagttaaatcagtaatctgtcgcatttcatcaagattaattaaaatttgggacctgcttcattcaagcttcatatatgctttgcagagaactcataaaggagcatataaggctaaatgtaaaacccaagactgtcattagaattgaattattgggcttaatataaatcgtaacctatgaagtttattttttattttagttaactatgattccaattactactttgttattgtacctaagtaaattttctttaagtcagaagcccattaaaatagttacaagcattgaacttctttagtattatattaatataaaaacatttttgtatgttttattgtaatcataaatactgctgtataaggtaataaaactctgcacctaatccccataacttccagtatcattttccaattaattatcaagtctgttttgggaaacactttgaggacatttatgatgcagcagatgttgactaaaggcttggttggtagatattcaggaaatgttcactgaataaataagtaaatacattattgaaaagcaaatctgtataaatgtgaaatttttatttgtattagtaataaaacattagtagtttaaacaaaaaaaaaaaaaaaaaaaaaaaaaaaaaaactcgactctagatt

SEQ ID NO: 4 - DNA, 289 bp, synthetic construct
  misc_feature 1..289: note = PAH 3'UTR sequence (289 nucleotides)
  source 1..289: mol_type = other DNA
agccatggacagaatgtggtctgtcagctgtgaatctgttgatggagatccaactatttctttcatcagaaaaagtccgaaaagcaaaccttaatttgaaataacagccttaaatcctttacaagatggagaaacaacaaataagtcaaaataatctgaaatgacaggatatgagtacatactcaagagcataatggtaaatcttttggggtcatctttgatttagagatgataatcccatactctcaattgagttaaatcagtaatctgtcgcatttcatcaagatta

SEQ ID NO: 5 - DNA, 53 bp, synthetic construct
  misc_feature 1..53: note = PAH shRNA sequence #1
  source 1..53: mol_type = other DNA
tcgcatttcatcaagattaatctcgagattaatcttgatgaaatgcgattttt

SEQ ID NO: 6 - DNA, 53 bp, synthetic construct
  misc_feature 1..53: note = PAH shRNA sequence #2
  source 1..53: mol_type = other DNA
actcataaaggagcatataagctcgagcttatatgctcctttatgagtttttt

SEQ ID NO: 7 - DNA, 228 bp, synthetic construct
  misc_feature 1..228: note = Rous Sarcoma virus (RSV) promoter
  source 1..228: mol_type = other DNA
gtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacg

SEQ ID NO: 8 - DNA, 180 bp, synthetic construct
  misc_feature 1..180: note = 5' Long terminal repeat (LTR)
  source 1..180: mol_type = other DNA
ggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 9 - DNA, 41 bp, synthetic construct
  misc_feature 1..41: note = Psi Packaging signal
  source 1..41: mol_type = other DNA
tacgccaaaaattttgactagcggaggctagaaggagagag

SEQ ID NO: 10 - DNA, 233 bp, synthetic construct
  misc_feature 1..233: note = Rev response element (RRE)
  source 1..233: mol_type = other DNA
aggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcc

SEQ ID NO: 11 - DNA, 118 bp, synthetic construct
  misc_feature 1..118: note = Central polypurine tract (cPPT)
  source 1..118: mol_type = other DNA
ttttaaaagaaaaggggggattggggggtacagtgcaggggaaagaatagtagacataatagcaacagacatacaaactaaagaattacaaaaacaaattacaaaattcaaaatttta

SEQ ID NO: 12 - DNA, 397 bp, synthetic construct
  misc_feature 1..397: note = Human alpha-1 antitrypsin promoter (hAAT)
  source 1..397: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 13 - DNA, 590 bp, synthetic construct
  misc_feature 1..590: note = Long WPRE sequence
  source 1..590: mol_type = other DNA
aatcaacctctgattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 14 - DNA, 250 bp, synthetic construct
  misc_feature 1..250: note = 3' delta LTR
  source 1..250: mol_type = other DNA
tggaagggctaattcactcccaacgaagataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtca

SEQ ID NO: 15 - DNA, 217 bp, synthetic construct
  misc_feature 1..217: note = H1 Promoter
  source 1..217: mol_type = other DNA
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccactt

SEQ ID NO: 16 - DNA, 642 bp, synthetic construct
  misc_feature 1..642: note = CAG promoter
  source 1..642: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 17 - DNA, 1503 bp, synthetic construct
  misc_feature 1..1503: note = HIV Gag
  source 1..1503: mol_type = other DNA
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 18 - DNA, 1872 bp, synthetic construct
  misc_feature 1..1872: note = HIV Pol
  source 1..1872: mol_type = other DNA
atgaatttgccaggaagatggaaaccaaaaatgatagggggaattggaggttttatcaaagtaggacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaaatatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagatgggttggtcagtgctggaatcaggaaagtacta

SEQ ID NO: 19 - DNA, 867 bp, synthetic construct
  misc_feature 1..867: note = HIV Int
  source 1..867: mol_type = other DNA
tttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 20 - DNA, 234 bp, synthetic construct
  misc_feature 1..234: note = HIV RRE
  source 1..234: mol_type = other DNA
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 21 - DNA, 351 bp, synthetic construct
  misc_feature 1..351: note = HIV Rev
  source 1..351: mol_type = other DNA
atggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatag

SEQ ID NO: 22 - DNA, 577 bp, synthetic construct
  misc_feature 1..577: note = CMV Promoter
  source 1..577: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagc

SEQ ID NO: 23 - DNA, 1531 bp, synthetic construct
  misc_feature 1..1531: note = VSV-G / DNA fragment containing VSV-G / Envelope Glycoprotein
  source 1..1531: mol_type = other DNA
gaattcatgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgagaattc

SEQ ID NO: 24 - DNA, 352 bp, synthetic construct
  misc_feature 1..352: note = CAG enhancer
  source 1..352: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatc

SEQ ID NO: 25 - DNA, 960 bp, synthetic construct
  misc_feature 1..960: note = Chicken beta actin intron
  source 1..960: mol_type = other DNA
ggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgg

SEQ ID NO: 26 - DNA, 448 bp, synthetic construct
  misc_feature 1..448: note = Rabbit beta globin poly A
  source 1..448: mol_type = other DNA
agatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatc

SEQ ID NO: 27 - DNA, 573 bp, synthetic construct
  misc_feature 1..573: note = Beta globin intron
  source 1..573: mol_type = other DNA
gtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacag

SEQ ID NO: 28 - DNA, 31 bp, synthetic construct
  misc_feature 1..31: note = Primer
  source 1..31: mol_type = other DNA
taagcagaattcatgaatttgccaggaagat

SEQ ID NO: 29 - DNA, 36 bp, synthetic construct
  misc_feature 1..36: note = Primer
  source 1..36: mol_type = other DNA
ccatacaatgaatggacactaggcggccgcacgaat

SEQ ID NO: 30 - DNA, 2745 bp, synthetic construct
  misc_feature 1..2745: note = Gag, Pol, Integrase fragment
  source 1..2745: mol_type = other DNA
gaattcatgaatttgccaggaagatggaaaccaaaaatgatagggggaattggaggttttatcaaagtaagacagtatgatcagatactcatagaaatctgcggacataaagctataggtacagtattagtaggacctacacctgtcaacataattggaagaaatctgttgactcagattggctgcactttaaattttcccattagtcctattgagactgtaccagtaaaattaaagccaggaatggatggcccaaaagttaaacaatggccattgacagaagaaaaaataaaagcattagtagaaatttgtacagaaatggaaaaggaaggaaaaatttcaaaaattgggcctgaaaatccatacaatactccagtatttgccataaagaaaaaagacagtactaaatggagaaaattagtagatttcagagaacttaataagagaactcaagatttctgggaagttcaattaggaataccacatcctgcagggttaaaacagaaaaaatcagtaacagtactggatgtgggcgatgcatatttttcagttcccttagataaagacttcaggaagtatactgcatttaccatacctagtataaacaatgagacaccagggattagatatcagtacaatgtgcttccacagggatggaaaggatcaccagcaatattccagtgtagcatgacaaaaatcttagagccttttagaaaacaaaatccagacatagtcatctatcaatacatggatgatttgtatgtaggatctgacttagaaatagggcagcatagaacaaaaatagaggaactgagacaacatctgttgaggtggggatttaccacaccagacaaaaaacatcagaaagaacctccattcctttggatgggttatgaactccatcctgataaatggacagtacagcctatagtgctgccagaaaaggacagctggactgtcaatgacatacagaaattagtgggaaaattgaattgggcaagtcagatttatgcagggattaaagtaaggcaattatgtaaacttcttaggggaaccaaagcactaacagaagtagtaccactaacagaagaagcagagctagaactggcagaaaacagggagattctaaaagaaccggtacatggagtgtattatgacccatcaaaagacttaatagcagaaatacagaagcaggggcaaggccaatggacatatcaaatttatcaagagccatttaaaaatctgaaaacaggaaagtatgcaagaatgaagggtgcccacactaatgatgtgaaacaattaacagaggcagtacaaaaaatagccacagaaagcatagtaatatggggaaagactcctaaatttaaattacccatacaaaaggaaacatgggaagcatggtggacagagtattggcaagccacctggattcctgagtgggagtttgtcaatacccctcccttagtgaagttatggtaccagttagagaaagaacccataataggagcagaaactttctatgtagatggggcagccaatagggaaactaaattaggaaaagcaggatatgtaactgacagaggaagacaaaaagttgtccccctaacggacacaacaaatcagaagactgagttacaagcaattcatctagctttgcaggattcgggattagaagtaaacatagtgacagactcacaatatgcattgggaatcattcaagcacaaccagataagagtgaatcagagttagtcagtcaaataatagagcagttaataaaaaaggaaaaagtctacctggcatgggtaccagcacacaaaggaattggaggaaatgaacaagtagataaattggtcagtgctggaatcaggaaagtactatttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaa

SEQ ID NO: 31 - DNA, 1586 bp, synthetic construct
  misc_feature 1..1586: note = DNA Fragment containing Rev, RRE and rabbit beta globin poly A
  source 1..1586: mol_type = other DNA
tctagaatggcaggaagaagcggagacagcgacgaagagctcatcagaacagtcagactcatcaagcttctctatcaaagcaacccacctcccaatcccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcagcggccgccccggg

SEQ ID NO: 32 - DNA, 1614 bp, synthetic construct
  misc_feature 1..1614: note = DNA fragment containing the CAG enhancer/promoter/intron sequence
  source 1..1614: mol_type = other DNA
acgcgttagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggaattc

SEQ ID NO: 33 - DNA, 884 bp, synthetic construct
  misc_feature 1..884: note = RSV promoter and HIV Rev
  source 1..884: mol_type = other DNA
caattgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattccgcattgcagagataattgtatttaagtgcctagctcgatacaataaacgccatttgaccattcaccacattggtgtgcacctccaagctcgagctcgtttagtgaaccgtcagatcgcctggagacgccatccacgctgttttgacctccatagaagacaccgggaccgatccagcctcccctcgaagctagcgattaggcatctcctatggcaggaagaagcggagacagcgacgaagaactcctcaaggcagtcagactcatcaagtttctctatcaaagcaacccacctcccaatcccgaggggacccgacaggcccgaaggaatagaagaagaaggtggagagagagacagagacagatccattcgattagtgaacggatccttagcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtctaga

SEQ ID NO: 34 - DNA, 1104 bp, synthetic construct
  misc_feature 1..1104: note = Elongation Factor-1 alpha (EF1-alpha) promoter
  source 1..1104: mol_type = other DNA
ccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 35 - DNA, 511 bp, synthetic construct
  misc_feature 1..511: note = Promoter-PGK
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 36 - DNA, 1162 bp, synthetic construct
  misc_feature 1..1162: note = Promoter-UbC
  source 1..1162: mol_type = other DNA
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaa

SEQ ID NO: 37 - DNA, 120 bp, synthetic construct
  misc_feature 1..120: note = Poly A-SV40
  source 1..120: mol_type = other DNA
gtttattgcagcttataatggttacaaataaagcaatagcatcacaaatttcacaaataaagcatttttttcactgcattctagttgtggtttgtccaaactcatcaatgtatcttatca

SEQ ID NO: 38 - DNA, 227 bp, synthetic construct
  misc_feature 1..227: note = Poly A-bGH
  source 1..227: mol_type = other DNA
gactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 39 - DNA, 1695 bp, synthetic construct
  misc_feature 1..1695: note = Envelope-RD114
  source 1..1695: mol_type = other DNA
atgaaactcccaacaggaatggtcattttatgtagcctaataatagttcgggcagggtttgacgacccccgcaaggctatcgcattagtacaaaaacaacatggtaaaccatgcgaatgcagcggagggcaggtatccgaggccccaccgaactccatccaacaggtaacttgcccaggcaagacggcctacttaatgaccaaccaaaaatggaaatgcagagtcactccaaaaaatctcacccctagcgggggagaactccagaactgcccctgtaacactttccaggactcgatgcacagttcttgttatactgaataccggcaatgcagggcgaataataagacatactacacggccaccttgcttaaaatacggtctgggagcctcaacgaggtacagatattacaaaaccccaatcagctcctacagtccccttgtaggggctctataaatcagcccgtttgctggagtgccacagcccccatccatatctccgatggtggaggacccctcgatactaagagagtgtggacagtccaaaaaaggctagaacaaattcataaggctatgcatcctgaacttcaataccaccccttagccctgcccaaagtcagagatgaccttagccttgatgcacggacttttgatatcctgaataccacttttaggttactccagatgtccaattttagccttgcccaagattgttggctctgtttaaaactaggtacccctacccctcttgcgatacccactccctctttaacctactccctagcagactccctagcgaatgcctcctgtcagattatacctcccctcttggttcaaccgatgcagttctccaactcgtcctgtttatcttcccctttcattaacgatacggaacaaatagacttaggtgcagtcacctttactaactgcacctctgtagccaatgtcagtagtcctttatgtgccctaaacgggtcagtcttcctctgtggaaataacatggcatacacctatttaccccaaaactggacaggactttgcgtccaagcctccctcctccccgacattgacatcatcccgggggatgagccagtccccattcctgccattgatcattatatacatagacctaaacgagctgtacagttcatccctttactagctggactgggaatcaccgcagcattcaccaccggagctacaggcctaggtgtctccgtcacccagtatacaaaattatcccatcagttaatatctgatgtccaagtcttatccggtaccatacaagatttacaagaccaggtagactcgttagctgaagtagttctccaaaataggaggggactggacctactaacggcagaacaaggaggaatttgtttagccttacaagaaaaatgctgtttttatgctaacaagtcaggaattgtgagaaacaaaataagaaccctacaagaagaattacaaaaacgcagggaaagcctggcatccaaccctctctggaccgggctgcagggctttcttccgtacctcctacctctcctgggacccctactcaccctcctactcatactaaccattgggccatgcgttttcaatcgattggtccaatttgttaaagacaggatctcagtggtccaggctctggttttgactcagcaatatcaccagctaaaacccatagagtacgagccatga

SEQ ID NO: 40 - DNA, 2013 bp, synthetic construct
  misc_feature 1..2013: note = Envelope-GALV
  source 1..2013: mol_type = other DNA
atgcttctcacctcaagcccgcaccaccttcggcaccagatgagtcctgggagctggaaaagactgatcatcctcttaagctgcgtattcggagacggcaaaacgagtctgcagaataagaacccccaccagcctgtgaccctcacctggcaggtactgtcccaaactggggacgttgtctgggacaaaaaggcagtccagcccctttggacttggtggccctctcttacacctgatgtatgtgccctggcggccggtcttgagtcctgggatatcccgggatccgatgtatcgtcctctaaaagagttagacctcctgattcagactatactgccgcttataagcaaatcacctggggagccatagggtgcagctaccctcgggctaggaccaggatggcaaattcccccttctacgtgtgtccccgagctggccgaacccattcagaagctaggaggtgtggggggctagaatccctatactgtaaagaatggagttgtgagaccacgggtaccgtttattggcaacccaagtcctcatgggacctcataactgtaaaatgggaccaaaatgtgaaatgggagcaaaaatttcaaaagtgtgaacaaaccggctggtgtaaccccctcaagatagacttcacagaaaaaggaaaactctccagagattggataacggaaaaaacctgggaattaaggttctatgtatatggacacccaggcatacagttgactatccgcttagaggtcactaacatgccggttgtggcagtgggcccagaccctgtccttgcggaacagggacctcctagcaagcccctcactctccctctctccccacggaaagcgccgcccacccctctacccccggcggctagtgagcaaacccctgcggtgcatggagaaactgttaccctaaactctccgcctcccaccagtggcgaccgactctttggccttgtgcagggggccttcctaaccttgaatgctaccaacccaggggccactaagtcttgctggctctgtttgggcatgagccccccttattatgaagggatagcctcttcaggagaggtcgcttatacctccaaccatacccgatgccactggggggcccaaggaaagcttaccctcactgaggtctccggactcgggtcatgcatagggaaggtgcctcttacccatcaacatctttgcaaccagaccttacccatcaattcctctaaaaaccatcagtatctgctcccctcaaaccatagctggtgggcctgcagcactggcctcaccccctgcctctccacctcagtttttaatcagtctaaagacttctgtgtccaggtccagctgatcccccgcatctattaccattctgaagaaaccttgttacaagcctatgacaaatcaccccccaggtttaaaagagagcctgcctcacttaccctagctgtcttcctggggttagggattgcggcaggtataggtactggctcaaccgccctaattaaagggcccatagacctccagcaaggcctaaccagcctccaaatcgccattgacgctgacctccgggcccttcaggactcaatcagcaagctagaggactcactgacttccctatctgaggtagtactccaaaataggagaggccttgacttactattccttaaagaaggaggcctctgcgcggccctaaaagaagagtgctgtttttatgtagaccactcaggtgcagtacgagactccatgaaaaaacttaaagaaagactagataaaagacagttagagcgccagaaaaaccaaaactggtatgaagggtggttcaataactccccttggtttactaccctactatcaaccatcgctgggcccctattgctcctccttttgttactcactcttgggccctgcatcatcaataaattaatccaattcatcaatgataggataagtgcagtcaaaattttagtccttagacagaaatatcagaccctagataacgaggaaaacctttaa

SEQ ID NO: 41 - DNA, 1530 bp, synthetic construct
  misc_feature 1..1530: note = Envelope-FUG
  source 1..1530: mol_type = other DNA
atggttccgcaggttcttttgtttgtactccttctgggtttttcgttgtgtttcgggaagttccccatttacacgataccagacgaacttggtccctggagccctattgacatacaccatctcagctgtccaaataacctggttgtggaggatgaaggatgtaccaacctgtccgagttctcctacatggaactcaaagtgggatacatctcagccatcaaagtgaacgggttcacttgcacaggtgttgtgacagaggcagagacctacaccaactttgttggttatgtcacaaccacattcaagagaaagcatttccgccccaccccagacgcatgtagagccgcgtataactggaagatggccggtgaccccagatatgaagagtccctacacaatccataccccgactaccactggcttcgaactgtaagaaccaccaaagagtccctcattatcatatccccaagtgtgacagatttggacccatatgacaaatcccttcactcaagggtcttccctggcggaaagtgctcaggaataacggtgtcctctacctactgctcaactaaccatgattacaccatttggatgcccgagaatccgagaccaaggacaccttgtgacatttttaccaatagcagagggaagagagcatccaacgggaacaagacttgcggctttgtggatgaaagaggcctgtataagtctctaaaaggagcatgcaggctcaagttatgtggagttcttggacttagacttatggatggaacatgggtcgcgatgcaaacatcagatgagaccaaatggtgccctccagatcagttggtgaatttgcacgactttcgctcagacgagatcgagcatctcgttgtggaggagttagttaagaaaagagaggaatgtctggatgcattagagtccatcatgaccaccaagtcagtaagtttcagacgtctcagtcacctgagaaaacttgtcccagggtttggaaaagcatataccatattcaacaaaaccttgatggaggctgatgctcactacaagtcagtccggacctggaatgagatcatcccctcaaaagggtgtttgaaagttggaggaaggtgccatcctcatgtgaacggggtgtttttcaatggtataatattagggcctgacgaccatgtcctaatcccagagatgcaatcatccctcctccagcaacatatggagttgttggaatcttcagttatccccctgatgcaccccctggcagacccttctacagttttcaaagaaggtgatgaggctgaggattttgttgaagttcacctccccgatgtgtacaaacagatctcaggggttgacctgggtctcccgaactggggaaagtatgtattgatgactgcaggggccatgattggcctggtgttgatattttccctaatgacatggtgcagagttggtatccatctttgcattaaattaaagcacaccaagaaaagacagatttatacagacatagagatgaaccgacttggaaagtaa

SEQ ID NO: 42 - DNA, 1497 bp, synthetic construct
  misc_feature 1..1497: note = Envelope-LCMV
  source 1..1497: mol_type = other DNA
atgggtcagattgtgacaatgtttgaggctctgcctcacatcatcgatgaggtgatcaacattgtcattattgtgcttatcgtgatcacgggtatcaaggctgtctacaattttgccacctgtgggatattcgcattgatcagtttcctacttctggctggcaggtcctgtggcatgtacggtcttaagggacccgacatttacaaaggagtttaccaatttaagtcagtggagtttgatatgtcacatctgaacctgaccatgcccaacgcatgttcagccaacaactcccaccattacatcagtatggggacttctggactagaattgaccttcaccaatgattccatcatcagtcacaacttttgcaatctgacctctgccttcaacaaaaagacctttgaccacacactcatgagtatagtttcgagcctacacctcagtatcagagggaactccaactataaggcagtatcctgcgacttcaacaatggcataaccatccaatacaacttgacattctcagatcgacaaagtgctcagagccagtgtagaaccttcagaggtagagtcctagatatgtttagaactgccttcggggggaaatacatgaggagtggctggggctggacaggctcagatggcaagaccacctggtgtagccagacgagttaccaatacctgattatacaaaatagaacctgggaaaaccactgcacatatgcaggtccttttgggatgtccaggattctcctttcccaagagaagactaagttcttcactaggagactagcgggcacattcacctggactttgtcagactcttcaggggtggagaatccaggtggttattgcctgaccaaatggatgattcttgctgcagagcttaagtgtttcgggaacacagcagttgcgaaatgcaatgtaaatcatgatgccgaattctgtgacatgctgcgactaattgactacaacaaggctgctttgagtaagttcaaagaggacgtagaatctgccttgcacttattcaaaacaacagtgaattctttgatttcagatcaactactgatgaggaaccacttgagagatctgatgggggtgccatattgcaattactcaaagttttggtacctagaacatgcaaagaccggcgaaactagtgtccccaagtgctggcttgtcaccaatggttcttacttaaatgagacccacttcagtgatcaaatcgaacaggaagccgataacatgattacagagatgttgaggaaggattacataaagaggcaggggagtacccccctagcattgatggaccttctgatgttttccacatctgcatatctagtcagcatcttcctgcaccttgtcaaaataccaacacacaggcacataaaaggtggctcatgtccaaagccacaccgattaaccaacaaaggaatttgtagttgtggtgcatttaaggtgcctggtgtaaaaaccgtctggaaaagacgctga

SEQ ID NO: 43 - DNA, 1692 bp, synthetic construct
  misc_feature 1..1692: note = Envelope-FPV
  source 1..1692: mol_type = other DNA
atgaacactcaaatcctggttttcgcccttgtggcagtcatccccacaaatgcagacaaaatttgtcttggacatcatgctgtatcaaatggcaccaaagtaaacacactcactgagagaggagtagaagttgtcaatgcaacggaaacagtggagcggacaaacatccccaaaatttgctcaaaagggaaaagaaccactgatcttggccaatgcggactgttagggaccattaccggaccacctcaatgcgaccaatttctagaattttcagctgatctaataatcgagagacgagaaggaaatgatgtttgttacccggggaagtttgttaatgaagaggcattgcgacaaatcctcagaggatcaggtgggattgacaaagaaacaatgggattcacatatagtggaataaggaccaacggaacaactagtgcatgtagaagatcagggtcttcattctatgcagaaatggagtggctcctgtcaaatacagacaatgctgctttcccacaaatgacaaaatcatacaaaaacacaaggagagaatcagctctgatagtctggggaatccaccattcaggatcaaccaccgaacagaccaaactatatgggagtggaaataaactgataacagtcgggagttccaaatatcatcaatcttttgtgccgagtccaggaacacgaccgcagataaatggccagtccggacggattgattttcattggttgatcttggatcccaatgatacagttacttttagtttcaatggggctttcatagctccaaatcgtgccagcttcttgaggggaaagtccatggggatccagagcgatgtgcaggttgatgccaattgcgaaggggaatgctaccacagtggagggactataacaagcagattgccttttcaaaacatcaatagcagagcagttggcaaatgcccaagatatgtaaaacaggaaagtttattattggcaactgggatgaagaacgttcccgaaccttccaaaaaaaggaaaaaaagaggcctgtttggcgctatagcagggtttattgaaaatggttgggaaggtctggtcgacgggtggtacggtttcaggcatcagaatgcacaaggagaaggaactgcagcagactacaaaagcacccaatcggcaattgatcagataaccggaaagttaaatagactcattgagaaaaccaaccagcaatttgagctaatagataatgaattcactgaggtggaaaagcagattggcaatttaattaactggaccaaagactccatcacagaagtatggtcttacaatgctgaacttcttgtggcaatggaaaaccagcacactattgatttggctgattcagagatgaacaagctgtatgagcgagtgaggaaacaattaagggaaaatgctgaagaggatggcactggttgctttgaaatttttcataaatgtgacgatgattgtatggctagtataaggaacaatacttatgatcacagcaaatacagagaagaagcgatgcaaaatagaatacaaattgacccagtcaaattgagtagtggctacaaagatgtgatactttggtttagcttcggggcatcatgctttttgcttcttgccattgcaatgggccttgttttcatatgtgtgaagaacggaaacatgcggtgcactatttgtatataa

SEQ ID NO: 44 - DNA, 1266 bp, synthetic construct
  misc_feature 1..1266: note = Envelope-RRV
  source 1..1266: mol_type = other DNA
agtgtaacagagcactttaatgtgtataaggctactagaccatacctagcacattgcgccgattgcggggacgggtacttctgctatagcccagttgctatcgaggagatccgagatgaggcgtctgatggcatgcttaagatccaagtctccgcccaaataggtctggacaaggcaggcacccacgcccacacgaagctccgatatatggctggtcatgatgttcaggaatctaagagagattccttgagggtgtacacgtccgcagcgtgctccatacatgggacgatgggacacttcatcgtcgcacactgtccaccaggcgactacctcaaggtttcgttcgaggacgcagattcgcacgtgaaggcatgtaaggtccaatacaagcacaatccattgccggtgggtagagagaagttcgtggttagaccacactttggcgtagagctgccatgcacctcataccagctgacaacggctcccaccgacgaggagattgacatgcatacaccgccagatataccggatcgcaccctgctatcacagacggcgggcaacgtcaaaataacagcaggcggcaggactatcaggtacaactgtacctgcggccgtgacaacgtaggcactaccagtactgacaagaccatcaacacatgcaagattgaccaatgccatgctgccgtcaccagccatgacaaatggcaatttacctctccatttgttcccagggctgatcagacagctaggaaaggcaaggtacacgttccgttccctctgactaacgtcacctgccgagtgccgttggctcgagcgccggatgccacctatggtaagaaggaggtgaccctgagattacacccagatcatccgacgctcttctcctataggagtttaggagccgaaccgcacccgtacgaggaatgggttgacaagttctctgagcgcatcatcccagtgacggaagaagggattgagtaccagtggggcaacaacccgccggtctgcctgtgggcgcaactgacgaccgagggcaaaccccatggctggccacatgaaatcattcagtactattatggactataccccgccgccactattgccgcagtatccggggcgagtctgatggccctcctaactctggcggccacatgctgcatgctggccaccgcgaggagaaagtgcctaacaccgtacgccctgacgccaggagcggtggtaccgttgacactggggctgctttgctgcgcaccgagggcgaatgca

SEQ ID NO: 45 - DNA, 1938 bp, synthetic construct
  misc_feature 1..1938: note = Envelope-MLV 10A1
  source 1..1938: mol_type = other DNA
atggaaggtccagcgttctcaaaaccccttaaagataagattaacccgtggaagtccttaatggtcatgggggtctatttaagagtagggatggcagagagcccccatcaggtctttaatgtaacctggagagtcaccaacctgatgactgggcgtaccgccaatgccacctcccttttaggaactgtacaagatgccttcccaagattatattttgatctatgtgatctggtcggagaagagtgggacccttcagaccaggaaccatatgtcgggtatggctgcaaataccccggagggagaaagcggacccggacttttgacttttacgtgtgccctgggcataccgtaaaatcggggtgtggggggccaagagagggctactgtggtgaatggggttgtgaaaccaccggacaggcttactggaagcccacatcatcatgggacctaatctcccttaagcgcggtaacaccccctgggacacgggatgctccaaaatggcttgtggcccctgctacgacctctccaaagtatccaattccttccaaggggctactcgagggggcagatgcaaccctctagtcctagaattcactgatgcaggaaaaaaggctaattgggacgggcccaaatcgtggggactgagactgtaccggacaggaacagatcctattaccatgttctccctgacccgccaggtcctcaatatagggccccgcatccccattgggcctaatcccgtgatcactggtcaactacccccctcccgacccgtgcagatcaggctccccaggcctcctcagcctcctcctacaggcgcagcctctatagtccctgagactgccccaccttctcaacaacctgggacgggagacaggctgctaaacctggtagaaggagcctatcaggcgcttaacctcaccaatcccgacaagacccaagaatgttggctgtgcttagtgtcgggacctccttattacgaaggagtagcggtcgtgggcacttataccaatcattctaccgccccggccagctgtacggccacttcccaacataagcttaccctatctgaagtgacaggacagggcctatgcatgggagcactacctaaaactcaccaggccttatgtaacaccacccaaagtgccggctcaggatcctactaccttgcagcacccgctggaacaatgtgggcttgtagcactggattgactccctgcttgtccaccacgatgctcaatctaaccacagactattgtgtattagttgagctctggcccagaataatttaccactcccccgattatatgtatggtcagcttgaacagcgtaccaaatataagagggagccagtatcgttgaccctggcccttctgctaggaggattaaccatgggagggattgcagctggaatagggacggggaccactgccctaatcaaaacccagcagtttgagcagcttcacgccgctatccagacagacctcaacgaagtcgaaaaatcaattaccaacctagaaaagtcactgacctcgttgtctgaagtagtcctacagaaccgaagaggcctagatttgctcttcctaaaagagggaggtctctgcgcagccctaaaagaagaatgttgtttttatgcagaccacacgggactagtgagagacagcatggccaaactaagggaaaggcttaatcagagacaaaaactatttgagtcaggccaaggttggttcgaagggcagtttaatagatccccctggtttaccaccttaatctccaccatcatgggacctctaatagtactcttactgatcttactctttggaccctgcattctcaatcgattggtccaatttgttaaagacaggatctcagtggtccaggctctggttttgactcaacaatatcaccagctaaaacctatagagtacgagccatga

SEQ ID NO: 46 - DNA, 2030 bp, synthetic construct
  misc_feature 1..2030: note = Envelope-Ebola
  source 1..2030: mol_type = other DNA
atgggtgttacaggaatattgcagttacctcgtgatcgattcaagaggacatcattctttctttgggtaattatccttttccaaagaacattttccatcccacttggagtcatccacaatagcacattacaggttagtgatgtcgacaaactggtttgccgtgacaaactgtcatccacaaatcaattgagatcagttggactgaatctcgaagggaatggagtggcaactgacgtgccatctgcaactaaaagatggggcttcaggtccggtgtcccaccaaaggtggtcaattatgaagctggtgaatgggctgaaaactgctacaatcttgaaatcaaaaaacctgacgggagtgagtgtctaccagcagcgccagacgggattcggggcttcccccggtgccggtatgtgcacaaagtatcaggaacgggaccgtgtgccggagactttgccttccacaaagagggtgctttcttcctgtatgaccgacttgcttccacagttatctaccgaggaacgactttcgctgaaggtgtcgttgcatttctgatactgccccaagctaagaaggacttcttcagctcacaccccttgagagagccggtcaatgcaacggaggacccgtctagtggctactattctaccacaattagatatcaagctaccggttttggaaccaatgagacagagtatttgttcgaggttgacaatttgacctacgtccaacttgaatcaagattcacaccacagtttctgctccagctgaatgagacaatatatacaagtgggaaaaggagcaataccacgggaaaactaatttggaaggtcaaccccgaaattgatacaacaatcggggagtgggccttctgggaaactaaaaaaacctcactagaaaaattcgcagtgaagagttgtctttcacagctgtatcaaacagagccaaaaacatcagtggtcagagtccggcgcgaacttcttccgacccagggaccaacacaacaactgaagaccacaaaatcatggcttcagaaaattcctctgcaatggttcaagtgcacagtcaaggaagggaagctgcagtgtcgcatctgacaacccttgccacaatctccacgagtcctcaaccccccacaaccaaaccaggtccggacaacagcacccacaatacacccgtgtataaacttgacatctctgaggcaactcaagttgaacaacatcaccgcagaacagacaacgacagcacagcctccgacactccccccgccacgaccgcagccggacccctaaaagcagagaacaccaacacgagcaagggtaccgacctcctggaccccgccaccacaacaagtccccaaaaccacagcgagaccgctggcaacaacaacactcatcaccaagataccggagaagagagtgccagcagcgggaagctaggcttaattaccaatactattgctggagtcgcaggactgatcacaggcgggaggagagctcgaagagaagcaattgtcaatgctcaacccaaatgcaaccctaatttacattactggactactcaggatgaaggtgctgcaatcggactggcctggataccatatttcgggccagcagccgagggaatttacatagaggggctgatgcacaatcaagatggtttaatctgtgggttgagacagctggccaacgagacgactcaagctcttcaactgttcctgagagccacaaccgagctacgcaccttttcaatcctcaaccgtaaggcaattgatttcttgctgcagcgatggggcggcacatgccacattttgggaccggactgctgtatcgaaccacatgattggaccaagaacataacagacaaaattgatcagattattcatgattttgttgataaaacccttccggaccagggggacaatgacaattggtggacaggatggagacaatggataccggcaggtattggagttacaggcgttataattgcagttatcgctttattctgtatatgcaaatttgtcttttag

SEQ ID NO: 47 - DNA, 141 bp, synthetic construct
  misc_feature 1..141: note = Left ITR
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 48 - DNA, 120 bp, synthetic construct
  misc_feature 1..120: note = Prothrombin enhancer
  source 1..120: mol_type = other DNA
gcgagaacttgtgcctccccgtgttcctgctctttgtccctctgtcctacttagactaatatttgccttgggtactgcaaacaggaaatgggggagggacaggagtagggcggagggtag

SEQ ID NO: 49 - DNA, 228 bp, synthetic construct
  misc_feature 1..228: note = PolyA
  source 1..228: mol_type = other DNA
gactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 50 - DNA, 141 bp, synthetic construct
  misc_feature 1..141: note = Right ITR
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 51 - DNA, 1470 bp, synthetic construct
  misc_feature 1..1470: note = E2A
  source 1..1470: mol_type = other DNA
ttaaaagtcgaaggggttctcgcgctcgtcgttgtgcgccgcgctggggagggccacgttgcggaactggtacttgggctgccacttgaactcggggatcaccagtttgggcactggggtctcggggaaggtctcgctccacatgcgccggctcatctgcagggcgcccagcatgtcaggcgcggagatcttgaaatcgcagttggggccggtgctctgcgcgcgcgagttgcggtacactgggttgcagcactggaacaccatcagactggggtacttcacactagccagcacgctcttgtcgctgatctgatccttgtccaggtcctcggcgttgctcaggccgaacggggtcatcttgcacagctggcggcccaggaagggcacgctctgaggcttgtggttacactcgcagtgcacgggcatcagcatcatccccgcgccgcgctgcatattcgggtagagggccttgacgaaggccgcgatctgcttgaaagcttgctgggccttggccccctcgctgaaaaacaggccgcagctcttcccgctgaactgattattcccgcacccggcatcatggacgcagcagcgcgcgtcatggctggtcagttgcaccacgctccgtccccagcggttctgggtcaccttggccttgctgggttgctccttcagcgcacgctgcccgttctcactggtcacatccatctccaccacgtggtccttgtggatcatcaccgtcccatgcagacacttgagctggccttccacctcggtgcagccgtggtcccacagggcactgccggtgcactcccagttcttgtgcgcgatcccgctgtggctgaagatgtaaccttgcaacaggcgacccatgatggtgctaaagctcttctgggtggtgaaggtcagttgcagaccgcgggcctcctcgttcatccaggtctggcacatcttttggaagatctcggtctgctcgggcatgagcttgtaagcatcgcgcaggccgctgtcgacgcggtagcgttccatcagcacattcatggtatccatgcccttctcccaggacgagaccagaggcagactcagggggttgcgcacgttcaggacaccgggggtcgcgggctcgacgatgcgttttccgtccttgccttccttcaacagaaccggcggctggctgaatcccactcccacgatcacggcttcttcctggggcatctcttcgtctgggtctaccttggtcacatgcttggtctttctggcttgcttcttttttggagggctgtccacggggaccacgtcctcctcggaagacccggatcccacccgctgatactttcggcgcttggttggcagaggaggtggcggcgaggggctcctctcctgctccggcggatagcgcgctgaaccgtggccccggggcggagtggcctctcggtccatgaaccggcgcacgtcctgactgccgccggccat

SEQ ID NO: 52 - DNA, 393 bp, synthetic construct
  misc_feature 1..393: note = E4
  source 1..393: mol_type = other DNA
tcatgtatctttattgatttttacaccagcacgggtagtcagtctcccaccaccagcccatttcacagtgtaaacaattctctcagcacgggtggccttaaatagggcaatattctgattagtgcgggaactggacttggggtctataatccacacagtttcctggcgagccaaacgggggtcggtgattgagatgaagccgtcctctgaaaagtcatccaagcgagcctcacagtccaaggtcacagtattatgataatctgcatgatcacaatcgggcaacaggggatgttgttcagtcagtgaagccctggtttcctcatcagatcgtggtaaacgggccctgcgatatggatgatggcggagcgagctggattgaatctcggtttgcat

SEQ ID NO: 53 - DNA, 162 bp, synthetic construct
  misc_feature 1..162: note = VA RNA
  source 1..162: mol_type = other DNA
agcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttt

SEQ ID NO: 54 - DNA, 2208 bp, synthetic construct
  misc_feature 1..2208: note = AAV2 Rep
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 55 - DNA, 1866 bp, synthetic construct
  misc_feature 1..1866: note = AAV2 Cap
  source 1..1866: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 56 - DNA, 1227 bp, synthetic construct
  misc_feature 1..1227: note = DNA Fragment containing RRE and rabbit beta globin poly A
  source 1..1227: mol_type = other DNA
tctagaaggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgccatatgctggctgccatgaacaaaggtggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatgaagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcacccggg

SEQ ID NO: 57 - DNA, 2312 bp, synthetic construct
  misc_feature 1..2312: note = hPAH with fill 5' and 3' UTR
  source 1..2312: mol_type = other DNA
ggcacgaggtacctgaggccctaaaaagccagagacctcactcccggggagccagcatgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtagctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaaagccatggacagaatgtggtctgtcagctgtgaatctgttgatggagatccaactatttctttcatcagaaaaagtccgaaaagcaaaccttaatttgaaataacagccttaaatcctttacaagatggagaaacaacaaataagtcaaaataatctgaaatgacaggatatgagtacatactcaagagcataatggtaaatcttttggggtcatctttgatttagagatgataatcccatactctcaattgagttaaatcagtaatctgtcgcatttcatcaagattaattaaaatttgggacctgcttcattcaagcttcatatatgctttgcagagaactcataaaggagcatataaggctaaatgtaaaacccaagactgtcattagaattgaattattgggcttaatataaatcgtaacctatgaagtttattttttattttagttaactatgattccaattactactttgttattgtacctaagtaaattttctttaagtcagaagcccattaaaatagttacaagcattgaacttctttagtattatattaatataaaaacatttttgtatgttttattgtaatcataaatactgctgtataaggtaataaaactctgcacctaatccccataacttccagtatcattttccaattaattatcaagtctgttttgggaaacactttgaggacatttatgatgcagcagatgttgactaaaggcttggttggtagatattcaggaaatgttcactgaataaataagtaaatacattattgaaaagcaaatctgtataaatgtgaaatttttatttgtattagtaataaaacattagtagtttaaacaaaaaaaaaaaaaaaaaaaaaaaaaaaaaaactcgactctagatt

SEQ ID NO: 58 - DNA, 1704 bp, synthetic construct
  misc_feature 1..1704: note = hPAH with full 5' UTR and truncated 3' UTR
  source 1..1704: mol_type = other DNA
ggcacgaggtacctgaggccctaaaaagccagagacctcactcccggggagccagcatgtccactgcggtcctggaaaacccaggcttgggcaggaaactctctgactttggacaggaaacaagctatattgaagacaactgcaatcaaaatggtgccatatcactgatcttctcactcaaagaagaagttggtgcattggccaaagtattgcgcttatttgaggagaatgatgtaaacctgacccacattgaatctagaccttctcgtttaaagaaagatgagtatgaatttttcacccatttggataaacgtagcctgcctgctctgacaaacatcatcaagatcttgaggcatgacattggtgccactgtccatgagctttcacgagataagaagaaagacacagtgccctggttcccaagaaccattcaagagctggacagatttgccaatcagattctcagctatggagcggaactggatgctgaccaccctggttttaaagatcctgtgtaccgtgcaagacggaagcagtttgctgacattgcctacaactaccgccatgggcagcccatccctcgagtggaatacatggaggaagaaaagaaaacatggggcacagtgttcaagactctgaagtccttgtataaaacccatgcttgctatgagtacaatcacatttttccacttcttgaaaagtactgtggcttccatgaagataacattccccagctggaagacgtttctcagttcctgcagacttgcactggtttccgcctccgacctgtagctggcctgctttcctctcgggatttcttgggtggcctggccttccgagtcttccactgcacacagtacatcagacatggatccaagcccatgtatacccccgaacctgacatctgccatgagctgttgggacatgtgcccttgttttcagatcgcagctttgcccagttttcccaggaaattggccttgcctctctgggtgcacctgatgaatacattgaaaagctcgccacaatttactggtttactgtggagtttgggctctgcaaacaaggagactccataaaggcatatggtgctgggctcctgtcatcctttggtgaattacagtactgcttatcagagaagccaaagcttctccccctggagctggagaagacagccatccaaaattacactgtcacggagttccagcccctgtattacgtggcagagagttttaatgatgccaaggagaaagtaaggaactttgctgccacaatacctcggcccttctcagttcgctacgacccatacacccaaaggattgaggtcttggacaatacccagcagcttaagattttggctgattccattaacagtgaaattggaatcctttgcagtgccctccagaaaataaagtaaagccatggacagaatgtggtctgtcagctgtgaatctgttgatggagatccaactatttctttcatcagaaaaagtccgaaaagcaaaccttaatttgaaataacagccttaaatcctttacaagatggagaaacaacaaataagtcaaaataatctgaaatgacaggatatgagtacatactcaagagcataatggtaaatcttttggggtcatctttgatttagagatgataatcccatactctcaattgagttaaatcagtaatctgtcgcatttcatcaagatta